SEQUENCE LISTING (27 sequences)

SEQ ID NO: 1 - RNA, 1112 nt, synthetic construct
  misc_feature 1..1112: note = Segment (nucleotides 1379-2468) of SARS-CoV2 S-gene
  source 1..1112: mol_type = other RNA
gccgcggatccggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattggaattccggcc

SEQ ID NO: 2 - DNA, 33 bp, synthetic construct
  misc_feature 1..33: note = Cy5 labeled; note = DNA sequence complementary to nucleotides 598-630 of SEQ ID NO: 1 (nucleotides 1976-2008 of SARS-CoV2 S-gene)
  source 1..33: mol_type = other DNA
ggattgactagctacactacgtgcccgccgagg

SEQ ID NO: 3 - DNA, 22 bp, synthetic construct
  misc_feature 1..22: note = DNA sequence complementary to nucleotides 1069-1090 of SEQ ID NO: 1 (nucleotides 2447-2468 of SARS-CoV2 S-gene); note = Cy5 labeled
  source 1..22: mol_type = other DNA
caatatcaccaaggcaatcacc

SEQ ID NO: 4 - DNA, 22 bp, synthetic construct
  misc_feature 1..22: note = DNA sequence complementary to nucleotides 43-64 of SEQ ID NO: 1 (nucleotides 1421-1442 of SARS-CoV2 S-gene); note = Cy5 labeled
  source 1..22: mol_type = other DNA
caccattagtgggttggaaacc

SEQ ID NO: 5 - RNA, 1869 nt, Homo sapiens
  source 1..1869: mol_type = rRNA
tacctggttgatcctgccagtagcatatgcttgtctcaaagattaagccatgcatgtctgagtacgcacggccggtacagtgaaactgcgaatggctcattaaatcagttatggttcctttggtcgctcgctcctctcctacttggataactgtggtaattctagagctaatacatgccgacgggcgctgacccccttcgcgggggggatgcgtgcatttatcagatcaaaaccaacccggtcagcccctctccggccccggccggggggcgggcgccggcggctttggtgactctagataacctcgggccgatcgcacgccccccgtggcggcgacgacccattcgaacgtctgccctatcaactttcgatggtagtcgccgtgcctaccatggtgaccacgggtgacggggaatcagggttcgattccggagagggagcctgagaaacggctaccacatccaaggaaggcagcaggcgcgcaaattacccactcccgacccggggaggtagtgacgaaaaataacaatacaggactctttcgaggccctgtaattggaatgagtccactttaaatcctttaacgaggatccattggagggcaagtctggtgccagcagccgcggtaattccagctccaatagcgtatattaaagttgctgcagttaaaaagctcgtagttggatcttgggagcgggcgggcggtccgccgcgaggcgagccaccgcccgtccccgccccttgcctctcggcgccccctcgatgctcttagctgagtgtcccgcggggcccgaagcgtttactttgaaaaaattagagtgttcaaagcaggcccgagccgcctggataccgcagctaggaataatggaataggaccgcggttctattttgttggttttcggaactgaggccatgattaagagggacggccgggggcattcgtattgcgccgctagaggtgaaattcttggaccggcgcaagacggaccagagcgaaagcatttgccaagaatgttttcattaatcaagaacgaaagtcggaggttcgaagacgatcagataccgtcgtagttccgaccataaacgatgccgaccggcgatgcggcggcgttattcccatgacccgccgggcagcttccgggaaaccaaagtctttgggttccggggggagtatggttgcaaagctgaaacttaaaggaattgacggaagggcaccaccaggagtggagcctgcggcttaatttgactcaacacgggaaacctcacccggcccggacacggacaggattgacagattgatagctctttctcgattccgtgggtggtggtgcatggccgttcttagttggtggagcgatttgtctggttaattccgataacgaacgagactctggcatgctaactagttacgcgacccccgagcggtcggcgtcccccaacttcttagagggacaagtggcgttcagccacccgagattgagcaataacaggtctgtgatgcccttagatgtccggggctgcacgcgcgctacactgactggctcagcgtgtgcctaccctacgccggcaggcgcgggtaacccgttgaaccccattcgtgatggggatcggggattgcaattattccccatgaacgaggaattcccagtaagtgcgggtcataagcttgcgttgattaggtcgaacttgactatctagaggaagtaaaagtcgtaacaaggtttccgtaggtgaaagtccctgccctttgtacacaccgcccgtcgctactaccgattggatggtttagtgaggccctcggatcggccccgccggggtcggcccacggccctggcggagcgctgagaagaccctgcggaaggatcatta

SEQ ID NO: 6 - DNA, 46 bp, synthetic construct
  misc_feature 1..46: note = ATTO488 labeled; note = DNA sequence complementary to nucleotides 205-250 of SEQ ID NO: 5
  source 1..46: mol_type = other DNA
aggggctgaccgggttggttttgatctgataaatgcacgcatcccc

SEQ ID NO: 7 - RNA, 1112 nt, synthetic construct
  misc_feature 1..1112: note = Segment (nucleotides 482-1593) of Influenza A H1N1 HA Gene
  source 1..1112: mol_type = other RNA
tcacgctggagcaaaaagcttctacaaaaacttgatatggctggttaaaaaaggaaattcatacccaaagctcaaccaaacctacattaatggtaaagggaaagaagtcctcgtgctgtggggcattcaccatccatctactactgctgaccaacaaagtctctatcagaatgcagatgcatatgtttttgtggggacatcaagatacagcaagaagttcaagccggaaatagcaacaagacccaaagtgagggatcaagaagggagaatgaactattactggacactagtagagccgggagacaaaataacattcgaagcaactggaaatctagtggtaccgagatatgcattcacaatggaaagaaatgctggatctggtattatcatttcagatacaccagtccacgattgcaatacaacttgtcagacacccgagggtgctataaacaccagcctcccatttcagaatgtacatccgatcacaattgggaaatgtccaaagtatgtaaaaagcacaaaattgagactggccacaggattgaggaatgtcccgtctattcaatctagaggcctattcggggccattgccggcttcattgaaggggggtggacagggatggtagatggatggtacggttatcaccatcaaaatgagcaggggtcaggatatgcagccgatctgaagagcacacaaaatgccattgataagattactaacaaagtaaattctgttattgaaaagatgaatacacagttcacagcagtgggtaaagagttcaaccaccttgaaaaaagaatagagaatctaaataaaaaagttgatgatggtttcctggacatttggacttacaatgccgaactgttggttctactggaaaatgaaagaactttggactatcacgattcaaatgtgaagaacttgtatgaaaaagtaagaaaccagttaaaaaacaatgccaaggaaattggaaacggctgctttgaattttaccacaaatgcgataacacatgcatggaaagtgtcaagaatgggacttatgactacccaaaatactcagaggaagcaaaattaaacagagaaaaaatagatggagtaaagctggaatcaa

SEQ ID NO: 8 - DNA, 33 bp, synthetic construct
  misc_feature 1..33: note = DNA sequence complementary to nucleotides 567-599 of SEQ ID NO: 7 (nucleotides 1048-1080 of Influenza A H1N1 HA gene; note = ATTO488 labeled
  source 1..33: mol_type = other DNA
tgaagccggcaatggccccgaataggcctctag

SEQ ID NO: 9 - DNA, 42 bp, synthetic construct
  misc_feature 1..42: note = DNA sequence complementary to nucleotides 1205-1244 of E. coli 16S rRNA; note = Cy5 labeled
  source 1..42: mol_type = other DNA
cccactcccatggtgtgacgggcggtgtgtacaaggcccggg

SEQ ID NO: 10 - DNA, 44 bp, synthetic construct
  misc_feature 1..44: note = Cy5 labeled; note = DNA sequence complementary to nucleotides 855-898 of E. coli 16S rRNA
  source 1..44: mol_type = other DNA
gcggccgtactccccaggcggtcgacttaacgcgttagctccgg

SEQ ID NO: 11 - DNA, 49 bp, synthetic construct
  misc_feature 1..49: note = DNA sequence complementary to nucleotides 313-361 of E. coli 16S rRNA; note = Cy5 labeled
  source 1..49: mol_type = other DNA
ccccactgctgcctcccgtaggagtctggaccgtgtctcagttccagtg

SEQ ID NO: 12 - DNA, 36 bp, synthetic construct
  misc_feature 1..36: note = DNA sequence complementary to nucleotides 1126-1161 of E. coli 16S rRNA; note = Cy5 labeled
  source 1..36: mol_type = other DNA
ggcagtctcctttgagttcccggccggaccgctggc

SEQ ID NO: 13 - DNA, 31 bp, synthetic construct
  misc_feature 1..31: note = DNA sequence complementary to nucleotides 375-405 of E. coli 16S rRNA; note = Cy5 labeled
  source 1..31: mol_type = other DNA
cacgcggcatggctgcatcaggcttgcgccc

SEQ ID NO: 14 - DNA, 42 bp, synthetic construct
  misc_feature 1..42: note = DNA sequence complementary to nucleotides 1030-1071 of E. coli 16S rRNA; note = Cy5 labeled
  source 1..42: mol_type = other DNA
cgagctgacgacagccatgcagcacctgtctcacggttcccg

SEQ ID NO: 15 - DNA, 45 bp, synthetic construct
  misc_feature 1..45: note = Cy5 labeled; note = DNA sequence complementary to nucleotides 103-147 of E. coli 16S rRNA
  source 1..45: mol_type = other DNA
ccccctccatcaggcagtttcccagacattactcacccgtccgcc

SEQ ID NO: 16 - DNA, 33 bp, synthetic construct; identical to SEQ ID NO: 2
  misc_feature 1..33: note = Cy5 labeled Single strand DNA oligonucleotide probe
  source 1..33: mol_type = other DNA

SEQ ID NO: 17 - DNA, 22 bp, synthetic construct
  misc_feature 1..22: note = Cy5 labeled Single strand DNA oligonucleotide probe
  source 1..22: mol_type = other DNA
caatatcaccaaggcaatcacc

SEQ ID NO: 18 - DNA, 22 bp, synthetic construct
  misc_feature 1..22: note = Cy5 labeled Single strand DNA oligonucleotide probe
  source 1..22: mol_type = other DNA
caccattagtgggttggaaacc

SEQ ID NO: 19 - DNA, 33 bp, synthetic construct; identical to SEQ ID NO: 8
  misc_feature 1..33: note = ATTO488 labeled Single strand DNA oligonucleotide probe
  source 1..33: mol_type = other DNA

SEQ ID NO: 20 - DNA, 46 bp, synthetic construct; identical to SEQ ID NO: 6
  misc_feature 1..46: note = ATTO488 labeled Single strand DNA oligonucleotide probe
  source 1..46: mol_type = other DNA

SEQ ID NO: 21 - DNA, 42 bp, synthetic construct; identical to SEQ ID NO: 9
  misc_feature 1..42: note = Cy5 labeled Single strand DNA oligonucleotide probe
  source 1..42: mol_type = other DNA

SEQ ID NO: 22 - DNA, 44 bp, synthetic construct; identical to SEQ ID NO: 10
  misc_feature 1..44: note = Cy5 labeled Single strand DNA oligonucleotide probe
  source 1..44: mol_type = other DNA

SEQ ID NO: 23 - DNA, 49 bp, synthetic construct; identical to SEQ ID NO: 11
  misc_feature 1..49: note = Cy5 labeled Single strand DNA oligonucleotide probe
  source 1..49: mol_type = other DNA

SEQ ID NO: 24 - DNA, 36 bp, synthetic construct; identical to SEQ ID NO: 12
  misc_feature 1..36: note = Cy5 labeled Single strand DNA oligonucleotide probe
  source 1..36: mol_type = other DNA

SEQ ID NO: 25 - DNA, 31 bp, synthetic construct; identical to SEQ ID NO: 13
  misc_feature 1..31: note = Cy5 labeled Single strand DNA oligonucleotide probe
  source 1..31: mol_type = other DNA

SEQ ID NO: 26 - DNA, 42 bp, synthetic construct; identical to SEQ ID NO: 14
  misc_feature 1..42: note = Cy5 labeled Single strand DNA oligonucleotide probe
  source 1..42: mol_type = other DNA

SEQ ID NO: 27 - DNA, 45 bp, synthetic construct; identical to SEQ ID NO: 15
  misc_feature 1..45: note = Cy5 labeled Single strand DNA oligonucleotide probe
  source 1..45: mol_type = other DNA

(19) United States
(12) Patent Application Publication
(10) Pub. No.: US 2025/0340939 A1
(43) Pub. Date: Nov. 6, 2025

(54) METHOD OF IDENTIFYING PRESENCE OF A NUCLEIC ACID

(71) Applicant: Bar-Ilan University, Ramat-Gan (IL)

(72) Inventors: Haim Yosef COHEN, Modiin-Maccabim-Reut (IL); Matan Yosef AVIVI, Rishon LeZion (IL); Noga TOUITOU, Beit El (IL)

(73) Assignee: Bar-Ilan University, Ramat-Gan (IL)

(21) Appl. No.: 18/713,668

(22) PCT Filed: Nov. 28, 2022
(86) PCT No.: PCT/IL2022/051263

§ 371 Date: May 27, 2024

Related U.S. Application Data
(60) Provisional application No. 63/283,483, filed on Nov. 28, 2021.

(51) Int. Cl. C12Q 1/6883; C12Q 1/6806; C12Q 1/6816

(52) U.S. Cl. CPC C12Q 1/6883; C12Q 1/6806; C12Q 1/6816

(57) ABSTRACT

Provided is a method of rapid and accurate qualitative and quantitative determination of a presence of a target nucleic acid in a sample effected by thermophoresis. Further described is a method of diagnosing a presence of a disease or disorder associated with a nucleic acid in a subject, the method comprising determining a presence of a target nucleic acid associated with the disease or disorder in a subject as described herein, wherein a presence of the target nucleic acid in the subject is indicative of a presence of the disease or disorder.

### RELATED APPLICATIONS

This application claims the benefit of priority of U.S. Provisional Patent Application No. 63/283,483 filed on Nov. 28, 2021, the contents of which are incorporated herein by reference in their entirety.

### SEQUENCE LISTING STATEMENT

The XML file entitled 100490 Replacement Sequence Listing.xml, created on Nov. 6, 2024, comprising 41,551 bytes is incorporated herein by reference. The sequence listing submitted herewith is identical to the sequence listing forming part of the international application.

### FIELD AND BACKGROUND OF THE INVENTION

The present invention, in some embodiments thereof, relates to diagnostics, and more particularly, but not exclusively, to a method of detecting a nucleic acid, such as, for example, a nucleic acid associated with a disease.

Diagnosis is an important step in controlling and/or treating viral infections. The primary method used for the detection of viral infections such as Covid-19 is based on the identification of the disease-associated ribonucleic acid (RNA) using quantitative polymerase chain reaction (qPCR), also known as real-time polymerase chain reaction.

In conventional RNA-based diagnosis using real-time polymerase chain reaction, RNA is first extracted from a biological sample such as nasopharyngeal swab. The extracted RNA is then converted into complementary DNA (cDNA) followed by DNA replication; both involving enzymatic reactions. If the target RNA is present in the sample, it will be amplified using specific primers designed for it. The detection occurs during DNA amplification, where the double stranded DNA (dsDNA) is detected (e.g., using TaqMan™ or SYBR™ Green probes and appropriate detection methods), in which the emitted fluorescence intensity is proportional to the amplified dsDNA concentration. Amplification and detection are typically carried out using well plates and a real-time polymerase chain reaction instrument.

Microscale thermophoresis (MST) is based on the detection of a temperature-induced change in fluorescence of a target, typically by using an infrared laser to apply a temperature gradient to a solution in a thin capillary. The change in fluorescence may be based on temperature-related intensity change, as well as by thermophoresis, the directed movement of particles in a microscopic temperature gradient. Analysis of the dependence of the MST signal on ligand concentration can be used to determine binding affinity.

Moon et al. [Biochemistry 2018, 57:4638-4643] describes the use of microscale thermophoresis to study interaction between RNA and peptides or small molecules.

Kurth et al. [Biosensors (Basel) 2019, 9:124] describe detection of VEGF using a VEGF-binding aptamer and thermophoresis.

Wienken et al. [Nucleic Acids Res 2011, 39:e52] describe thermophoresis measurements at various temperatures for obtaining melting curves for nucleic acids, wherein mutations can be observed as changed melting temperature due to a mismatch.

Jacob et al. [Angew. Chem. Int. Ed. 2019, 58:9565-9569] describes absolute quantification of noncoding RNA such as tRNA by microscale thermophoresis.

### SUMMARY OF THE INVENTION

According to an aspect of some embodiments of the invention, there is provided a method of determining a presence of at least one target nucleic acid in a sample, the method comprising:
(a) contacting a nucleic acid-containing fraction of the sample with at least one probe compound capable of binding to at least one of the target nucleic acids;
(b) exposing the probe compound to a temperature gradient; and
(c) detecting a signal of the probe compound during the exposing to the temperature gradient,
the signal being indicative of a presence or absence of the target nucleic acid in the sample.

In some embodiments, the method comprising contacting the nucleic acid-containing fraction of the sample with more than one probe compound capable of binding to the target nucleic acid.

In some embodiments, the method further comprising contacting the nucleic acid-containing fraction of the sample with an intercalating agent.

According to some of any of the embodiments of the invention, detecting the signal comprises determining a change in the signal with respect to a signal of the probe compound in the absence of the target nucleic acid, wherein a change that is beyond a predetermined threshold of the probe compound in the absence of the target nucleic acid is indicative of a presence of the target nucleic acid in the sample.

According to some embodiments of the present invention, the method is configured for quantitative determination of the target nucleic acid.

According to some of any of the embodiments of the invention, the target nucleic acid is a single-stranded nucleic acid.

According to some of any of the embodiments of the invention, the target nucleic acid comprises RNA.

According to some of any of the embodiments of the invention, the target nucleic acid is associated with cancer, a misfolded protein, a bacterial infection, a fungal infection, a yeast infection, a viral infection or any other including multicellular pathogens, as well as a genetic abnormality.

Hence according to some of any of the embodiments of the invention, the pathogen is a virus/virion, a prion, a bacterium/microbe, or a fungus/yeast or any other pathogen including a multicellular pathogen such as worms and other parasites.

According to some of any of the embodiments of the invention, the target nucleic acid is a pathogen-specific RNA. In some embodiments, the target nucleic acid is that of a mutated gene that causes cancer in a subject; in such embodiments where the subject is human, the pathogen-specific RNA is a human RNA.

In some embodiments, the pathogen-specific RNA is a ribosomal RNA (rRNA).

According to some of any of the embodiments of the invention, the probe compound comprises a nucleic acid having a sequence complementary to at least a portion of the target nucleic acid.

According to some of any of the embodiments of the invention relating to a nucleic acid comprised by a probe compound, the nucleic acid comprised by the probe compound comprises DNA.

According to some of any of the embodiments of the invention relating to a nucleic acid comprised by a probe compound, a GC % of the sequence complementary to at least a portion of the target nucleic acid is at least 54%.

According to some of any of the embodiments of the invention relating to a nucleic acid comprised by a probe compound, the sequence complementary to at least a portion of the target nucleic acid is at least 14 bases in length.

According to some of any of the embodiments of the invention relating to a nucleic acid comprised by a probe compound, a length of the nucleic acid comprised by the probe compound is no more than 1% of the length of the target nucleic acid.

According to some of any of the embodiments of the invention relating to a nucleic acid comprised by a probe compound, a length of the sequence complementary to at least a portion of the target nucleic acid is at least 14 bases and no more than 1% of the length of the target nucleic acid, and a GC % of the sequence complementary to at least a portion of the target nucleic acid is at least 90% of the highest possible GC % for a sequence of the aforementioned length complementary to at least a portion of the target nucleic acid.

According to some of any of the embodiments of the invention relating to a nucleic acid comprised by a probe compound, the nucleic acid comprised by the probe compound does not exhibit self-annealing or inter-loops.

According to some of any of the embodiments of the invention relating to a nucleic acid comprised by a probe compound, the nucleic acid comprised by the probe compound is selected to minimize homology with viral RNA, bacterial RNA and the human transcriptome.

According to some of any of the embodiments of the invention, the temperature gradient is generated using an infrared laser.

According to some of any of the embodiments of the invention, detecting the signal is effected at least one second, optionally from 1 to 60 seconds, after initial exposure of the probe compound to the temperature gradient.

In some of any of the respective embodiments described herein, detecting the signal is completed no more than 60 seconds after initial exposure of the probe compound to the temperature gradient.

According to some of any of the embodiments of the invention, a low temperature region of the temperature gradient comprises a temperature of about 25° C.

According to some of any of the embodiments of the invention, detecting the signal is at a high temperature region of the temperature gradient.

According to some of any of the embodiments of the invention, the signal is normalized to a signal of the probe compound in the absence of the temperature gradient.

According to some of any of the embodiments of the invention, the signal is a fluorescent signal.

According to some of any of the embodiments of the invention, the probe compound comprises a fluorescent label conjugated to a moiety capable of binding to the target nucleic acid.

According to some of any of the embodiments of the invention relating to a moiety capable of binding to the target nucleic acid, the moiety capable of binding to the target nucleic acid is a complementary nucleic acid, and a fluorescent label is conjugated to a 5-prime end of the complementary nucleic acid.

According to some of any of the embodiments of the invention relating to a fluorescent label, the fluorescent label is selected from the group consist of a cyanine dye and an ATTO 488 dye.

According to some of any of the embodiments of the invention relating to a fluorescent label, a fluorescent label is a cyanine dye, and detecting the signal comprises determining an increase in fluorescence in a high temperature region of the temperature gradient with respect to a signal of a probe compound in the absence of the target nucleic acid.

According to some of any of the embodiments of the invention relating to a fluorescent label, a fluorescent label is an ATTO 488 dye, and detecting the signal comprises determining a decrease in fluorescence in a high temperature region of the temperature gradient with respect to a signal of a probe compound in the absence of the target nucleic acid.

According to some of any of the embodiments of the invention, the method further comprises contacting the nucleic acid-containing fraction of the sample with a control probe compound capable of binding to a control nucleic acid, and detecting a signal of the control probe compound during exposure to the temperature gradient.

According to some of any of the embodiments of the invention relating to a control probe compound, the method comprises normalizing the signal of the probe compound to the signal of the control probe compound.

According to some of any of the embodiments of the invention relating to a control probe compound, the method comprises concomitantly detecting the signal of the probe compound and the signal of the control probe compound.

According to some of any of the embodiments of the invention, the method further comprises contacting the nucleic acid-containing fraction of the sample with an additional compound capable of binding to nucleic acids.

According to some of any of the embodiments of the invention relating to an additional compound capable of binding to nucleic acids, contacting with the additional compound is effected subsequently to contacting with the probe compound.

According to some of any of the embodiments of the invention relating to an additional compound capable of binding to nucleic acids, the additional compound capable of binding to nucleic acids comprises at least one intercalating agent.

According to some of any of the embodiments of the invention relating to an intercalating agent, the intercalating agent is selected from the group consisting of an anthracycline, an acridine dye and 4′,6-diamidino-2-phenylindole (DAPI).

According to some of any of the embodiments of the invention relating to an anthracycline, a concentration of the anthracycline is at least 50 μM.

According to some of any of the embodiments of the invention relating to an anthracycline, the anthracycline is doxorubicin.

According to some of any of the embodiments of the invention, contacting the nucleic acid-containing fraction of the sample with the probe compound is effected in the presence of formamide.

According to some of any of the embodiments of the invention relating to formamide, a concentration of formamide is in a range of from 1 to 12 weight percent.

According to some of any of the embodiments of the invention, contacting the nucleic acid-containing fraction of the sample with the probe compound is effected in the presence of a surfactant.

According to some of any of the embodiments of the invention relating to a surfactant, the surfactant is polysorbate 20 at a concentration in a range of from 0.005 to 0.5 weight percent.

According to some of any of the embodiments of the invention, contacting the nucleic acid-containing fraction of the sample with the probe compound is effected at a temperature in a range of from 32° C. to 82° C.

According to some of any of the embodiments of the invention, contacting the nucleic acid-containing fraction of the sample with the probe compound is effected for at least 5 minutes.

According to some of any of the embodiments of the invention, contacting the nucleic acid-containing fraction of the sample with the probe compound is effected at a pH in a range of from 6 to 8.

According to some of any of the embodiments of the invention, contacting the nucleic acid-containing fraction of the sample with the probe compound is effected in the presence of a citrate buffer, wherein a concentration of citrate in the buffer is in a range of from 7.5 mM to 120 mM.

According to some of any of the embodiments of the invention, contacting the nucleic acid-containing fraction of the sample with the probe compound is effected in a solution comprising at least one salt, wherein a total concentration of ions in the solution is in a range of from 0.3 mM to 3000 mM.

According to some of any of the embodiments of the invention, the method further comprises exposing the probe compound to a temperature of at least 80° C. prior to contacting with the nucleic acid-containing fraction of the sample.

According to some of any of the embodiments of the invention, the method further comprises concomitantly determining a presence of a first target nucleic acid in the sample using a first probe compound and a presence of a second target nucleic acid in the sample using a second probe compound.

According to some of any of the embodiments of the invention relating to use of a first probe compound and a second probe compound, the method comprises concomitantly detecting the signal of the first probe compound and the signal of the second probe compound.

According to some of any of the embodiments of the invention relating to use of a first probe compound and a second probe compound, a signal of the first probe compound is a fluorescent signal detected at a first wavelength and a signal of the second probe compound is a fluorescent signal detected at a second wavelength.

According to some of any of the embodiments of the invention, the sample is selected from the group consisting of a food sample, a water sample, an agricultural sample, and a biological sample obtained from a subject.

According to some of any of the embodiments of the invention, the sample is a biological sample of a subject, the method being for determining a presence of the target nucleic acid and/or of a disease or disorder associated with the target nucleic acid in the subject.

According to an aspect of some embodiments of the invention, there is provided a method of determining a presence of a target nucleic acid in a subject, the method comprising determining a presence of the target nucleic acid according to a method described herein, according to any of the respective embodiments, in a biological sample obtained from the subject.

According to some of any of the embodiments of the invention relating to a method of determining a presence of a target nucleic acid in a subject, the method is for determining a presence of a plurality of target nucleic acids in the subject, the method comprising determining a presence of each of the plurality of target nucleic acids in the biological sample according to a method comprising concomitantly determining a presence of a first target nucleic acid and a second target nucleic acid in a sample, according to any of the respective embodiments described herein.

According to an aspect of some embodiments of the invention, there is provided a method of diagnosing a presence of a disease or disorder associated with a nucleic acid in a subject, the method comprising determining a presence of a target nucleic acid associated with the disease or disorder in a subject according to a method described herein, according to any of the respective embodiments, wherein a presence of the target nucleic acid in the subject is indicative of a presence of the disease or disorder in the subject.

According to some of any of the embodiments of the invention relating to a method of diagnosing a presence of a disease or disorder, the method is for determining a presence or absence of a plurality of diseases or disorders associated with a nucleic acid in a subject, the method comprising detecting a presence or absence of a plurality of target nucleic acids in a subject according to a method for determining a presence of a plurality of target nucleic acids in the subject, according to any of the respective embodiments described herein, wherein each of the plurality of target nucleic acids is associated with a different disease or disorder.

According to some of any of the embodiments of the invention relating to a method of diagnosing a presence of a disease or disorder, the disease or disorder is associated with a cancer, a genetic abnormality, a microbe and/or virus.

According to some of any of the embodiments of the invention relating to a method of diagnosing a presence of a disease or disorder, the disease or disorder comprises a viral infection.

Unless otherwise defined, all technical and/or scientific terms used herein have the same meaning as commonly understood by one of ordinary skill in the art to which the invention pertains.

Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEW OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents a schematic depiction of a method according to some embodiments of the invention; RNA is extracted (e.g., using a guanidinium thiocyanate phenol-chloroform extraction technique) and then hybridized with a target specific fluorescent DNA probe, prior to microscale thermophoresis (MST) measurement.

FIG. 2 presents a schematic depiction of the spatial distribution of unbound Cy5-DNA probe and Cy5-DNA:RNA hybrid (Cy5-labeled DNA probe bound to target RNA) with (I.R ON) and without (I.R OFF) a temperature gradient (with temperature correlated to darkness), optionally generated by I.R. laser; in absence of temperature gradient, spatial distribution is random, whereas in presence of temperature gradient, probe migrates to cooler regions and unbound probe migrates more effectively.

FIG. 3 presents a representative data for unbound DNA probe and DNA:RNA hybrid, wherein normalized fluorescence after thermophoresis is calculated as the ratio of fluorescence determined between t=1 to 2 seconds (Fₕₒₜ) to fluorescence determined between t=−1 to 0 seconds (F_{cold}) (t=0, indicated by arrow, represents time of application of temperature gradient).

FIG. 4 presents a graph showing the normalized fluorescence (Fₙₒᵣₘ) of a Cy5-labelled DNA probe as a function of RNA concentration, after exposure to RNA extracted from U2-OS cells treated (+) or untreated (−) with doxycycline (n=2) or to a control with no RNA (n=8); the cells stably expressed MS2 bacteriophage RNA under a doxycycline expression system (Tet-On) and the probe was designed to target MS2 RNA;

FIG. 5 presents a scatter plot showing the normalized fluorescence (Fₙₒᵣₘ) (one-point measurements) of a Cy5-labelled DNA probe after exposure to RNA extracted from U2-OS cells treated (+) or untreated (−) with doxycycline (n=4) or to a control with no RNA (n=8); measurements were performed using 50% red LED power and 60% infrared laser of a Monolith™ MST instrument for 10 seconds (*** p<0.001, NS=not statistically significant, according to one-way ANOVA test).

FIGS. 6A and 6B present graphs showing the normalized fluorescence (Fₙₒᵣₘ) of S^{Cy5} probe as a function of S-gene RNA concentration (n=2 for each indicated concentration) following thermophoresis (FIG. 6A), and representative MST curves as a function of time upon thermophoresis (FIG. 6B).

FIGS. 7A and 7B present graphs showing the normalized fluorescence (Fₙₒᵣₘ) of 18S^{ATTO488} probe as a function of the total concentration of RNA extracted from HEK 293T cells, following hybridization at temperatures of 37° C., 42° C., 52° C. and 61° C. and thermophoresis (FIG. 7A), and as a function of time upon thermophoresis (FIG. 7B).

FIGS. 8A and 8B present graphs showing the normalized fluorescence (Fₙₒᵣₘ) of S^{Cy5} probe as a function of S-gene RNA concentration in a mixture of various concentrations of S-gene RNA with 100 ng/μl of RNA extracted from HEK 293T human cells, following hybridization (at 42° C.) for 30 or 60 minutes or overnight (FIG. 8A), and as a function of time upon thermophoresis, with black dashed line indicating control sample with no RNA and 30 minute hybridization time (FIG. 8B); signals obtained using red wavelength acquisition mode.

FIGS. 9A and 9B present graphs showing the normalized fluorescence (Fₙₒᵣₘ) of 18S^{ATTO488} probe as a function of S-gene RNA concentration in a mixture of various concentrations of S-gene RNA with 100 ng/μl of RNA extracted from HEK 293T cells, following hybridization (at 42° C.) for 30 or 60 minutes or overnight (FIG. 9A), and as a function of time upon thermophoresis, with black dashed line indicating control sample with no RNA and 30 minute hybridization time (FIG. 9B); signals obtained using blue wavelength acquisition mode.

FIG. 10 presents a scatter plot showing the normalized fluorescence (Fₙₒᵣₘ) of 18S^{ATTO488} probe in various samples following hybridization with 400 ng/μl S-gene, 400 ng/μl control RNA or 100 ng/μl RNA extracted from HEK 293T cells, or with no RNA; signals obtained using blue wavelength acquisition mode (*** p<0.001, n=8).

FIG. 11 presents a scatter plot showing the normalized fluorescence (Fₙₒᵣₘ) of S^{Cy5} probe in various samples following hybridization with 400 ng/μl S-gene, 400 ng/μl control RNA or 100 ng/μl RNA extracted from HEK 293T cells, or with no RNA; signals obtained using red wavelength acquisition mode (*** p<0.001, n=8).

FIG. 12 presents a schematic depiction of quantification of specific species of RNA within a mixture of RNA species, by specific binding of SARS-Cov2 S-gene RNA in a sample by an exemplary fluorescent DNA probe (S^{Cy5}) and specific binding of 18S rRNA by another exemplary fluorescent DNA probe (18S^{ATTO488}) FIGS. 13A and 13B present graphs showing the normalized fluorescence (Fₙₒᵣₘ) of 18S^{ATTO488} probe as a function of concentration of S-gene RNA (gray) or control RNA (black) following thermophoresis (FIG. 13A), and as a function of time upon thermophoresis (FIG. 13B); for each sample, S-gene RNA or control RNA was mixed with 100 ng/μl RNA extracted from HEK293T cells (signals obtained using blue wavelength acquisition mode, n=4).

FIGS. 14A and 14B present graphs showing the normalized fluorescence (Fₙₒᵣₘ) of S^{Cy5} probe as a function of concentration of S-gene RNA (gray) or control RNA (black) following thermophoresis (FIG. 14A), and as a function of time upon thermophoresis, with black dashed line indicating control sample with no RNA (FIG. 14B); for each sample, S-gene RNA or control RNA was mixed with 100 ng/μl RNA extracted from HEK293T cells (signals obtained using red wavelength acquisition mode, n=4).

FIG. 15 presents a graph showing the linear portion of the thermophoretic migration signal (ΔFₙₒᵣₘ) of S^{Cy5} as a function of S-gene RNA concentration (in a mixture with 100 ng/μl RNA extracted from HEK293T cells), as presented in FIG. 14A.

FIG. 16 presents a graph showing statistical distributions of the thermophoretic migration signal (ΔFₙₒᵣₘ) of S^{Cy5} from 8 different samples with 400 ng/μl S-gene RNA or with no RNA, as well as the Z′ factor (representing assay robustness).

FIG. 17 presents a graph showing the thermophoretic migration signal (ΔFₙₒᵣₘ) of S^{Cy5} as a function of S-gene RNA concentration, following hybridization of S^{Cy5} and S-gene RNA in SSC X2 buffer with 0%, 10%, 20% or 40% (v/v) formamide.

FIG. 18 presents a graph showing the thermophoretic migration signal (ΔFₙₒᵣₘ) of S^{Cy5} as a function of S-gene RNA concentration, following hybridization of S^{Cy5} and S-gene RNA in SSC buffer at various concentrations (X1, X2 or X4) (in the absence of formamide).

FIG. 19 presents a graph showing the thermophoretic migration signal (ΔFₙₒᵣₘ) of S^{Cy5} as a function of S-gene RNA concentration, following hybridization of S^{Cy5} and S-gene RNA in phosphate buffered saline (PBS) at various concentrations (X1, X2 or X4) (in the absence of formamide).

FIG. 20 presents a graph showing the thermophoretic migration signal (ΔFₙₒᵣₘ) of S^{Cy5} as a function of S-gene RNA concentration, following hybridization of S^{Cy5} and S-gene RNA in 50 mM Tris buffer with 150 mM NaCl or 50 mM trisodium citrate buffer with 150 mM NaCl (SSC X1) (in the absence of formamide).

FIG. 21 presents a graph showing the thermophoretic migration signal (ΔFₙₒᵣₘ) of S^{Cy5} as a function of S-gene RNA concentration, following hybridization of S^{Cy5} and S-gene RNA in pure water (in the absence of buffer or formamide).

FIG. 22 presents a graph showing the thermophoretic migration signal (ΔFₙₒᵣₘ) of S^{Cy5} as a function of S-gene RNA concentration, following hybridization of S^{Cy5} and S-gene RNA in water with 150 mM NaCl (in the absence of buffer or formamide).

FIG. 23 presents a graph showing the thermophoretic migration signal (ΔFₙₒᵣₘ) of S^{Cy5} as a function of S-gene RNA concentration, following hybridization of S^{Cy5} and S-gene RNA in 50 mM Tris buffer with 0, 0.15, 1.5, 15, 150 or 300 mM NaCl (in the absence of formamide).

FIG. 24 presents a graph showing the thermophoretic migration signal (ΔFₙₒᵣₘ) of S^{Cy5} as a function of S-gene RNA concentration, following hybridization of S^{Cy5} and S-gene RNA in 0.05 mM Tris buffer (in the absence of formamide).

FIG. 25 presents a graph showing the thermophoretic migration signal (ΔFₙₒᵣₘ) of S^{Cy5} as a function of S-gene RNA concentration, following hybridization of S^{Cy5} and S-gene RNA in 50 mM sodium phosphate buffer (in the absence of formamide).

FIG. 26 presents a schematic depiction of the effect of temperature gradient on the distribution of the exemplary S^{Cy5} probe and S^{Cy5}:S-gene DNA:RNA hybrid; S-gene is depicted is a narrow line, S^{Cy5} is depicted as a bold line with a star (representing Cy5), and salts are represented as spheres labeled + or −.

FIG. 27 presents an image of a gel electrophoresis of S-gene RNA (SEQ ID NO: 1), two batches (HA1 and HA2) of hemagglutinin RNA (SEQ ID NO: 7), and control RNA.

FIGS. 28A and 28B present graphs showing the thermophoretic migration signal (ΔFₙₒᵣₘ) of HA^{ATTO488} probe as a function of HA (H1N1 influenza hemagglutinin) gene concentration, in the presence of saline sodium citrate (SSC) buffer (X2) with 0%, 10%, 20% or 40% (v/v) formamide (FIG. 28A), and normalized fluorescence as a function of time upon thermophoresis for various samples, with arrows directed from high to low RNA concentrations (FIG. 28B); signals obtained using blue wavelength acquisition mode.

FIGS. 29A and 29B present graphs showing the thermophoretic migration signal (ΔFₙₒᵣₘ) of HA^{ATTO488} probe as a function of HA-gene concentration, in the presence of different concentrations (X1, X2 or X4) of saline sodium citrate (SSC) buffer (FIG. 29A), and normalized fluorescence as a function of time upon thermophoresis for various samples, with arrow directed from high to low RNA concentrations (FIG. 29B); signals obtained using blue wavelength acquisition mode.

FIGS. 30A and 30B present graphs showing the thermophoretic migration signal (ΔFₙₒᵣₘ) of HA^{ATTO488} probe as a function of RNA concentration, in the presence of HA-gene RNA, S-gene RNA or control (Ctrl) RNA, with saline sodium citrate buffer (X1) and 10% (v/v) formamide (FIG. 30A), and normalized fluorescence as a function of time upon thermophoresis for various samples in the presence of HA-gene RNA (gray solid lines), S-gene RNA (gray dashed lines) or control RNA (black dashed lines), with arrow directed from high to low RNA concentrations (FIG. 30B); signals obtained using blue wavelength acquisition mode.

FIGS. 31A and 31B presents a graph showing the thermophoretic migration signal (ΔFₙₒᵣₘ) of HA^{ATTO488} probe and S^{Cy5} probe as a function of RNA concentration, in the presence of a mixture of HA-gene RNA and S-gene RNA (S & HA), or control RNA (Ctrl), with saline sodium citrate buffer (X1) and 10% (v/v) formamide (FIG. 31A), and normalized fluorescence for red (dark gray) and blue (light gray) wavelengths as a function of time upon thermophoresis for various samples, with black dashed lines indicating control samples with no RNA (FIG. 31B); signals obtained using both red and blue wavelength acquisition modes.

FIGS. 32A and 32B present a graph showing the thermophoretic migration signal (ΔFₙₒᵣₘ) of HA^{ATTO488} probe and S^{Cy5} probe as a function of RNA concentration, in the presence of a mixture of HA-gene RNA and S-gene RNA (S & HA), or control RNA (Ctrl), with saline sodium citrate buffer (X1) and 1% (v/v) formamide (FIG. 32A), and normalized fluorescence for red (dark gray) and blue (light gray) wavelengths as a function of time upon thermophoresis for various samples, with black dashed lines indicating control samples with no RNA (FIG. 32B); each experiment performed using 4 replicates, signals obtained using both red and blue wavelength acquisition modes.

FIG. 33 presents a graph showing the linear portion of the thermophoretic migration signal (ΔFₙₒᵣₘ) for a mixture of HA^{ATTO488} probe and S^{Cy5} probe, obtained using the red acquisition mode, as presented in FIG. 32A.

FIG. 34 presents a graph showing the linear portion of the thermophoretic migration signal (ΔFₙₒᵣₘ) for a mixture of HA^{ATTO488} probe and S^{Cy5} probe, obtained using the blue acquisition mode, as presented in FIG. 32A.

FIG. 35 presents a schematic depiction of the spatial distribution of exemplary HA^{ATTO488} probes (with light star representing ATTO 488) and S^{Cy5} probe (with dark star representing Cy5), each in a form of unbound DNA probe and DNA:RNA hybrid (DNA probe bound to target RNA), in the presence of a temperature gradient (with temperature correlated to darkness), optionally generated by I.R. laser; S^{Cy5} probe migrates to cooler regions and unbound S^{Cy5} probe migrates more effectively, and HA^{ATTO488} probe migrates to warmer regions and unbound HA^{ATTO488} probe migrates less effectively.

FIG. 36 presents a schematic depiction of binding of S-gene RNA (long gray line) to exemplary fluorescent DNA probes (shorter black line) capable of binding to the end (S_{E}^{Cy5}) or to the middle (S^{Cy5}) of the S-gene RNA, as well as an exemplary fluorescent DNA probe (S_{N}^{Cy5}) which does not bind to of S-gene RNA (Cy5 fluorescent label of probes depicted as spheres).

FIG. 37 presents a graph showing the thermophoretic migration signal (ΔFₙₒᵣₘ) for various exemplary probes and combinations thereof as a function of S-gene RNA concentration.

FIGS. 38A and 38B presents a graph showing the linear portion of the thermophoretic migration signal (ΔFₙₒᵣₘ) for various exemplary probes and combinations thereof as a function of S-gene RNA concentration, at low RNA concentrations, as presented in FIG. 37 (FIG. 38A), and a bar graph showing the slopes of the linear regression models for each of the exemplary probes and combination thereof (FIG. 38B).

FIGS. 39A and 39B presents a graph showing the thermophoretic migration signal (ΔFₙₒᵣₘ) of Midori™ Green at dilutions of 1:1000, 1:2000, 1:4000, 1:8000 and 1:16000 as a function of S-gene RNA concentration in the presence of S^{Cy5} probe (FIG. 39A), and normalized fluorescence of Midori™ Green at a dilution of 1:1000 as a function of time upon thermophoresis for samples with various RNA concentrations (FIG. 39B); signals obtained using blue wavelength acquisition mode.

FIGS. 40A and 40B presents a graph showing the thermophoretic migration signal (ΔFₙₒᵣₘ) of S^{Cy5} probe as a function of S-gene RNA concentration in the presence of Midori™ Green (MG) at dilutions of 1:1000, 1:2000, 1:4000, 1:8000 and 1:16000 or with no Midori™ Green (FIG. 40A), and normalized fluorescence of S^{Cy5} with no Midori™ Green (black) or with Midori™ Green at a dilution of 1:1000 (gray), as a function of time upon thermophoresis for samples with various RNA concentrations (FIG. 40B); signals obtained using red wavelength acquisition mode.

FIGS. 41A, 41B and 41C presents graphs showing the linear portion of the thermophoretic migration signal (ΔFnorm) for S^{Cy5} probe in the presence (gray) or absence (black) of Midori™ Green at a 1:16000 dilution (FIG. 41A) or in the presence of Midori™ Green at dilutions of 1:1000 (black) or 1:8000 (gray) (FIG. 41B), as a function of S-gene RNA concentration (Midori™ Green was added after hybridization with S-gene RNA), and a bar graph showing the slopes of the linear regression models for the aforementioned samples (FIG. 41C); signals obtained using red wavelength acquisition mode.

FIGS. 42A and 42B present graphs showing the thermophoretic migration signal (ΔFₙₒᵣₘ) of 1, 10 or 100 μM Dox in the presence of S-gene RNA and S^{Cy5} probe (Dox added after hybridization of probe with S-gene RNA) as a function of RNA concentration (FIG. 42A), and normalized fluorescence as a function of time upon thermophoresis (FIG. 42B); signals obtained using blue wavelength acquisition mode.

FIGS. 43A and 43B present graphs showing the thermophoretic migration signal (ΔFₙₒᵣₘ) of S^{Cy5} probe in the presence (or absence) of 1, 10 or 100 μM Dox (added after hybridization with S-gene RNA) as a function of RNA concentration (FIG. 43A), and normalized fluorescence as a function of time upon thermophoresis, with dashed lines indicating samples without Dox (FIG. 43B); signals obtained using red wavelength acquisition mode.

FIG. 44 presents a graph showing initial fluorescence (F_{Initial}) of S^{Cy5} probe in the presence (or absence) of 1, 10 or 100 μM Dox, as a function of RNA concentration; signals obtained using red wavelength acquisition mode.

FIGS. 45A and 45B present a graph showing the linear portion of the thermophoretic migration signal (ΔFₙₒᵣₘ) for S^{Cy5} probe in the presence (or absence) of 1 μM, 10 μM or 100 μM Dox (added after hybridization with S-gene RNA), as a function of S-gene RNA concentration (FIG. 45A), and a bar graph showing the slopes of the linear regression models for the aforementioned samples (FIG. 45B); signals obtained using red wavelength acquisition mode.

FIG. 46 presents a graph showing the normalized fluorescence (Fₙₒᵣₘ) of 100 μM Dox in the presence of S-gene RNA, as a function of RNA concentration; signals obtained using blue wavelength acquisition mode.

FIG. 47 presents a graph showing the normalized fluorescence of 3.125, 6.25, 12.5, 25, 50 or 100 μM Dox, as a function of time upon thermophoresis; signals obtained using blue wavelength acquisition mode.

FIGS. 48A and 48B present graphs showing the thermophoretic migration signal (ΔFₙₒᵣₘ) of S^{Cy5} probe in the presence of 100 μM Dox (added after hybridization with S-gene RNA), as a function of RNA concentration following exposure to S-gene RNA (gray) or control RNA (black) and subsequent thermophoresis (FIG. 48A), and normalized fluorescence as a function of time upon thermophoresis (FIG. 48B); signals obtained using red wavelength acquisition mode.

FIGS. 49A and 49B present graphs showing the thermophoretic migration signal (ΔFₙₒᵣₘ) for 100 μM Dox added to S^{Cy5}, S_{N}^{Cy5} and S_{E}^{Cy5} probes or to a mixture of S^{Cy5} and S_{E}^{Cy5} probes (after hybridization of the probes with S-gene RNA), as a function of S-gene RNA concentration (FIG. 49A), and normalized fluorescence of S^{Cy5} (dark gray solid lines), S_{N}^{Cy5} (black solid lines) and S_{E}^{Cy5} (light gray solid lines) probes or a mixture of S^{Cy5} and S_{E}^{Cy5} probes (gray dashed lines) as a function of time upon thermophoresis, with black dashed line indicating control sample without S-gene RNA (FIG. 49B); signals obtained using blue wavelength acquisition mode.

FIG. 50 presents a graph showing the thermophoretic migration signal (ΔFₙₒᵣₘ) for S^{Cy5}, S_{N}^{Cy5} and S_{E}^{Cy5} probes and for a mixture of S^{Cy5} and S_{E}^{Cy5} probes in the presence of 100 μM Dox (added after hybridization with S-gene RNA), as a function of S-gene RNA concentration; signals obtained using red wavelength acquisition mode.

FIGS. 51A, 51B and 51C present graphs showing microscale thermophoresis curves at various S-gene RNA concentrations for S^{Cy5}, along with those of S_{N}^{Cy5} negative control (FIG. 51A; dark lines are S_{N}^{Cy5}, light gray are S^{Cy5}), S_{E}^{Cy5} (FIG. 51B; dark lines are S^{Cy5}, light gray lines are S_{E}^{cy5}), and a mixture of S^{Cy5} and S_{E}^{Cy5} (FIG. 51C; dashed lines are S^{Cy5}+S_{E}^{Cy5}).

FIGS. 52A and 52B present a graph showing the thermophoretic migration signal (ΔFₙₒᵣₘ) for S^{Cy5}, S_{N}^{Cy5} and S_{E}^{Cy5} probes and for a mixture of S^{Cy5} and S_{E}^{Cy5} probes in the presence of 100 μM Dox (added after hybridization with S-gene RNA), as a function of S-gene RNA concentration (FIG. 52A), and a bar graph showing the slopes of the linear regression models for the aforementioned probes (FIG. 52B); signals obtained using red wavelength acquisition mode.

FIGS. 53A and 53B present a graph showing the thermophoretic migration signal (ΔFₙₒᵣₘ) for S^{Cy5} or a mixture of S^{Cy5} and S_{E}^{Cy5} probes in the presence or absence of 100 μM Dox (added after hybridization with S-gene RNA), as a function of S-gene RNA concentration (FIG. 53A), and a bar graph showing the slopes of the linear regression models for the aforementioned samples (FIG. 53B); signals obtained using red wavelength acquisition mode.

FIGS. 54A and 54B present a graph showing the thermophoretic migration signal (ΔFₙₒᵣₘ) for a mixture of S^{Cy5} and S_{E}^{Cy5} probes in the presence or absence of 100 μM Dox (added after hybridization with S-gene RNA), as a function of S-gene RNA concentration (FIG. 54A), and a bar graph showing the slopes of the linear regression models for the aforementioned samples (FIG. 54B); signals obtained using red wavelength acquisition mode.

FIG. 55 presents a graph showing the thermophoretic migration signal (ΔFₙₒᵣₘ) for exemplary probes (S^{Cy5} and S_{E}^{Cy5}) in the presence of S-gene RNA and S-HA gene RNA, as a function of RNA concentration.

FIGS. 56A and 56B present a graph showing the linear portion of the thermophoretic migration signal (ΔFₙₒᵣₘ) for exemplary probes (S^{Cy5} and S_{E}^{Cy5}) in the presence of S-gene RNA and S-HA gene RNA, as a function of S-gene RNA concentration at low RNA concentrations, as presented in FIG. 55 (FIG. 56A) and a bar graph showing the slopes of the linear regression models for each combination of probe and target RNA (FIG. 56B).

FIG. 57 presents an image of an agarose gel showing two batches of RNA extracted from E. coli (DH5α strain).

FIGS. 58A and 58B present graphs showing the thermophoretic migration signal (ΔFₙₒᵣₘ) of 16S₁^{Cy5} probe as a function of the total concentration of bacterial RNA, following hybridization for 10 minutes at temperatures of 37° C., 42° C., 51° C., 56° C., 62° C., 71° C., 82° C. and 86° C. (FIG. 58A), and microscale thermophoresis curves for various RNA concentrations following hybridization at 71° C. (FIG. 58B).

FIGS. 59A and 59B present graphs showing the normalized fluorescence (Fₙₒᵣₘ) of 16S₁^{Cy5} probe as a function of the total concentration of RNA extracted from E. coli (DH5α strain) or human HEK 293T cells, following hybridization for 2 hours (FIG. 59A), and microscale thermophoresis curves (FIG. 59B).

FIGS. 60A, 60B and 60C present graphs showing the thermophoretic migration signal (ΔFₙₒᵣₘ) of 16S₁^{Cy5} probe as a function of the total concentration of RNA extracted from E. coli (DH5α strain) or human HEK 293T cells, following hybridization for 2 hours and then addition of 100 μM doxorubicin (FIG. 60A), microscale thermophoresis curves (FIG. 60B), and a graph showing a linear regression model for the aforementioned samples (FIG. 60C); signals obtained using red wavelength acquisition mode.

FIG. 61 presents a bar graph shows the ratio of sensitivity of thermophoretic migration signals of 16S₁^{Cy5} probe in the presence of Dox to the sensitivity in the absence of Dox; the sensitivity in each case was calculated by subtracting the thermophoretic migration signal (ΔFₙₒᵣₘ) in the presence of human RNA from the signal in the presence of bacterial RNA to obtain ΔΔFₙₒᵣₘ value.

FIGS. 62A and 62B present graphs showing the thermophoretic migration signal (ΔFₙₒᵣₘ) of doxorubicin (100 μM) in the presence of 16S₁^{Cy5} hybridized for 2 hours with RNA extracted from E. coli (DH5α strain) or human HEK 293T cells, as a function of the total concentration of RNA following hybridization for 2 hours and then addition of 100 μM doxorubicin and thermophoresis (FIG. 62A), and microscale thermophoresis curves (FIG. 62B) for bacterial RNA (dark lines) and human RNA (light lines); signals obtained using blue wavelength acquisition mode.

FIG. 63 presents a graph showing the thermophoretic migration signal (ΔFₙₒᵣₘ) of 16S₁^{Cy5} probe as a function of the total concentration of bacterial RNA, following hybridization for 0, 5, 30, 60 or 120 minutes with the RNA at 71° C., followed by addition of 100 μM doxorubicin and thermophoresis; signals obtained using red wavelength acquisition mode.

FIG. 64 presents a graph showing the thermophoretic migration signal (ΔFₙₒᵣₘ) of 16S₁^{Cy5}, 16S₂^{Cy5}, 16S₃^{Cy5}, 16S₄^{Cy5}, 16S₅^{Cy5}, 16S₆^{Cy5} and 16S₇^{Cy5} probes hybridized with bacterial DNA for 10 minutes, followed by addition of 100 μM doxorubicin and thermophoresis, as a function of RNA concentration; signals obtained using red wavelength acquisition mode.

FIG. 65 presents a graph showing the linear portion of the thermophoretic migration signal (ΔFnorm) data presented in FIG. 64.

FIG. 66 presents microscale thermophoresis curves for 16S₂^{Cy5} (light lines) or 16S₇^{Cy5} (dark lines) probes hybridized with bacterial DNA for 10 minutes, followed by addition of 100 μM doxorubicin; signals obtained using red wavelength acquisition mode.

FIG. 67 presents a graph showing the melting point (Tm) of 16S₁^{Cy5}, 16S₂^{Cy5}, 16S₃^{Cy5}, 16S₄^{Cy5}, 16S₅^{Cy5}, 16S₆^{Cy5} and 16S₇^{Cy5} probes as a function of the slopes of the linear regression models for each probe, as shown in FIG. 65.

FIG. 68 presents a bar graph showing the slopes of the linear regression models for the thermophoretic migration signal (ΔFₙₒᵣₘ) of 16S₇^{Cy5} probe alone (7) or in combination with 16S₁^{Cy5} (7+1), 16S₂^{Cy5} (7+2), 16S₃^{Cy5} (7+3), 16S₄^{Cy5} (7+4), 16S₅^{Cy5} (7+5) or 16S₆^{Cy5} (7+6) probes as a function of total bacterial RNA concentration, upon hybridization of probes for 10 minutes with RNA, followed by incubation with 100 μM doxorubicin; signals obtained using red wavelength acquisition mode.

FIG. 69 presents a bar graph showing the slopes of the linear regression models for the thermophoretic migration signal (ΔFₙₒᵣₘ) of 16S₇^{Cy5} probe alone (7) or in combination with one or more of 16S₁^{Cy5} (1), 16S₂^{Cy5} (2), 16S₃^{Cy5} (3), 16S₄^{Cy5} (4), 16S₅^{Cy5} (5) or 16S₆^{Cy5} (6) probes as a function of total bacterial RNA concentration, upon hybridization of probes for 10 minutes with RNA, followed by incubation with 100 μM doxorubicin; signals obtained using red wavelength acquisition mode.

FIG. 70 presents a bar graph showing the slopes of the linear regression models for the thermophoretic migration signal (ΔFₙₒᵣₘ) of 16S₇^{Cy5} probe alone (7) or in combination with one or more of 16S₁^{Cy5} (1), 16S₃^{Cy5} (3), 16S₄^{Cy5} (4), 16S₅^{Cy5} (5) or 16S₆^{Cy5} (6) probes as a function of total bacterial RNA concentration, upon hybridization of probes for 10 minutes with RNA, followed by incubation with 100 μM doxorubicin; signals obtained using red wavelength acquisition mode.

FIGS. 71A and 71B present graphs showing the thermophoretic migration signal (ΔFₙₒᵣₘ) of a mixture of 16S₃^{Cy5}, 16S₅^{Cy5}, 16S₆^{Cy5} and 16S₇^{Cy5} probes hybridized for 10 minutes with bacterial RNA, followed by incubation with 100 μM doxorubicin (+Dox) or hybridization buffer (−Dox), as a function of the total concentration of bacterial RNA (FIG. 71A), and microscale thermophoresis curves, with black dashed line indicating control sample with no RNA (FIG. 71B); signals obtained using red wavelength acquisition mode.

FIGS. 72A and 72B present a graph showing the linear portion of the thermophoretic migration signal (ΔFₙₒᵣₘ) a mixture of 16S₃^{Cy5}, 16S₅^{Cy5}, 16S₆^{Cy5} and 16S₇^{Cy5} probes in the presence or absence of Dox, as a function of total bacterial RNA concentration, as presented in FIG. 71A (FIG. 72A) and a bar graph showing the slopes of the linear regression models for each group (FIG. 72B).

FIGS. 73A-E present bacterial infections diagnosis by targeting 16S ribosomal RNA using MST; (FIG. 73A) The dose-dependent generated signals of 16S₁^{Cy5} probe and 16S_{7,6,5,3}^{Cy5} probe mix hybridized with increasing concentrations of total extracted RNA, followed by post-hybridization with 100 μM Dox or hybridization buffer as control; Measurements recorded using the red wavelength acquisition mode; Magnification of the responses without Dox are shown in the small graph; Error bars representing SD of different replicates n=2; (FIG. 73B) The linear regression model of (FIG. 73A); All MST curves of 16S_{7,6,5,3}^{Cy5} probe mix followed by post-hybridization incubation with 100 μM Dox (grey) or hybridization buffer as control (black) are shown in the upper graph; Error bars representing SD of different replicates n=2; (FIG. 73C) Representative standard curve of the generated signals of 16S_{7,6,5,3}^{Cy5} probe mix hybridized with increasing concentrations of total extracted RNA from 108 E. coli bacteria, followed by post-hybridization incubation with 100 μM Dox; The dashed line represents the starting of the competition between the DNA:RNA hybrid and free RNA on Dox, and characterized by Dox (black line) signal increase and Cy5 (grey line) signal decrease; MST curves are shown in the upper graph. (FIG. 73D) Bacterial load determination in 10 mL of natural urine samples spiked with pathogenic E. coli strains; The strains derived from three different human patients (n=3); No RNA control (n=16); Error bars representing SD of different samples; (FIG. 73E) Antimicrobial susceptibility testing (AST) of 5*10\hat{ }5 E. coli bacterial cultures grown for 90-150 minutes in the presence of 12.5 mg/mL ampicillin antibiotic. t0 n=2, t1.5 and t2.5 n=3; Error bars representing SD of independent cultures; *** p value<0.001 according to student t-test; Error bars representing SD of different cell cultures; F.I-Fluorescence intensity, t-Time in MST graphs.

FIGS. 74A-E present bacterial infections diagnosis by targeting 16S ribosomal RNA using MST; (FIG. 74A) The effect of hybridization time at 71° C. on the dose-dependent generated signals of 16S₁^{Cy5} probe hybridized with increasing concentrations of total bacterial RNA followed by post-hybridization with 100 μM Dox; signals recorded using the red wavelength acquisition mode; (FIG. 74B) The dose-dependent generated signals of 16S₇^{Cy5} in the presence and absence of 16S₁^{Cy5} hybridized with increasing concentrations of total RNA, followed by post-hybridization with 100 M Dox; Error bars describing SEM of two independent experiments; (FIG. 74C); The different linear regression models of FIG. 74C which are used for bacterial load determination; (FIGS. 74D-E) Two independent growth curves of E. coli; (FIG. 74D) Showing the increase in signal as represented by ΔFₙₒᵣₘ [‰] values; Growth started from 10⁵ bacteria (FIG. 74E) extrapolated bacteria values; Growth started from 10⁸ bacteria and optical density (O.D) measurements were preformed prior RNA extraction.

### DESCRIPTION OF SPECIFIC EMBODIMENTS OF THE INVENTION

The present invention, in some embodiments thereof, relates to diagnostics, and more particularly, but not exclusively, to a method of detecting a nucleic acid, such as, for example, a nucleic acid associated with a disease.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have envisioned that microscale thermophoresis can be adapted, using specially designed probes, to detect specific nucleic acids, and that such a method can be used to rapidly and accurately detect and/or quantify the presence of specific genes and/or organisms, at relatively low cost. The inventors have further envisioned that such methods may replace (for at least some applications) conventional RT-PCR methods, which are accurate, but are considerably time-consuming and costly.

While reducing the present invention to practice, the inventors have shown concentration-dependent detection of viral RNA, bacterial RNA and human RNA; detection times (following sample preparation) of as little as several seconds; the ability to concomitantly detect of RNA from two types of virus using probes specific for each RNA; and the use of a combination of multiple probes specific for the same type of RNA or a combination of a specific probe and an intercalating agent to enhance sensitivity.

Without being bound by any particular theory, it is believed that the method described herein may be less prone to workflow errors due to the lack of any need for cDNA conversion and DNA amplification, as in RT-PCT methods for RNA detection.

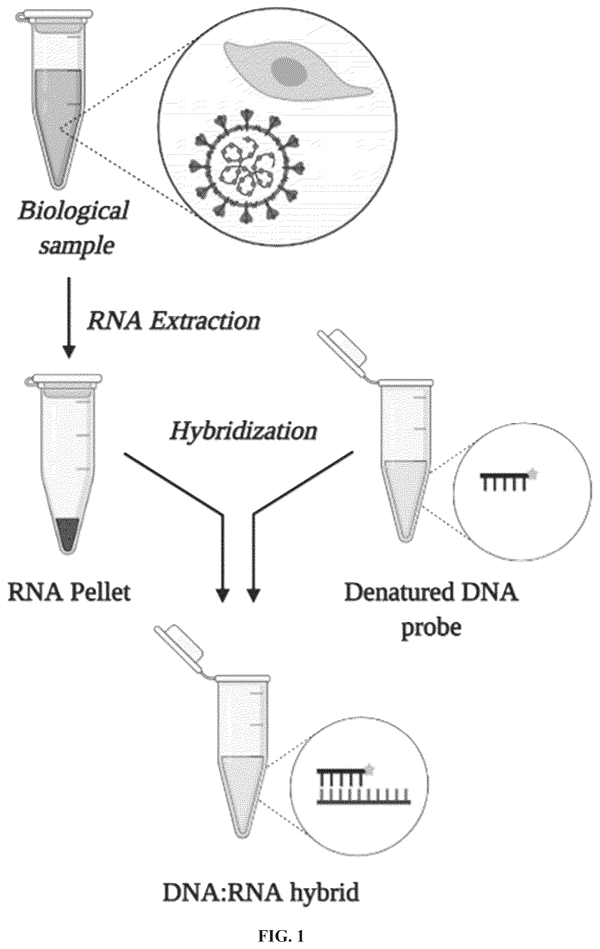

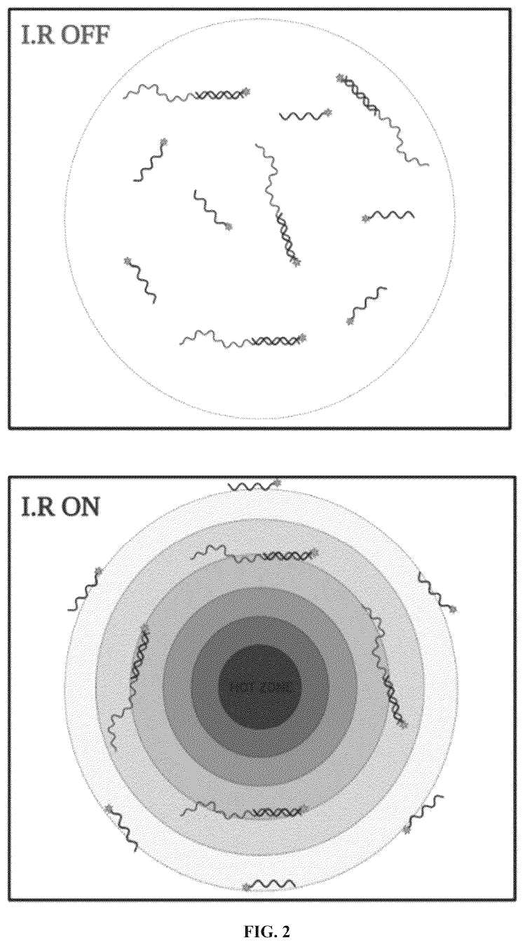

Referring to the drawings, FIG. 1 presents a schematic depiction of a method according to some embodiments of the invention. FIG. 2 presents a schematic depiction of the spatial distribution of an exemplary probe and probe-target nucleic acid hybrid with and without a temperature gradient.

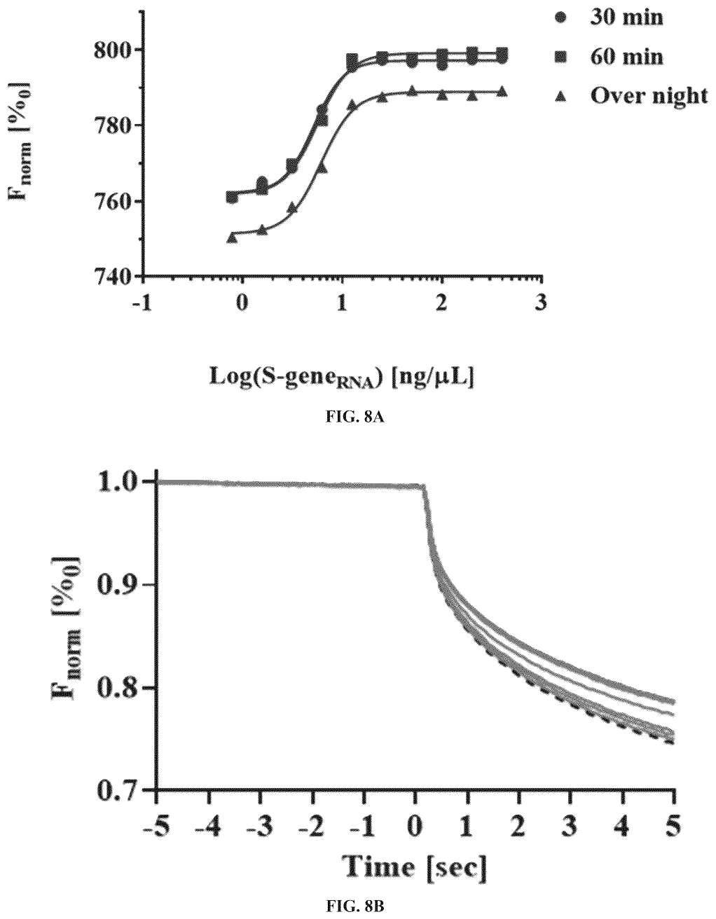

FIGS. 3-7B and 64-67 show the effect of the presence of a target nucleic acid (of various types) on a signal associated with a probe specific to the target nucleic acid. FIGS. 8A-16, 30A-30B and 59A-60C show the specificity of the probe-target nucleic acid interaction in the presence of other types of nucleic acid.

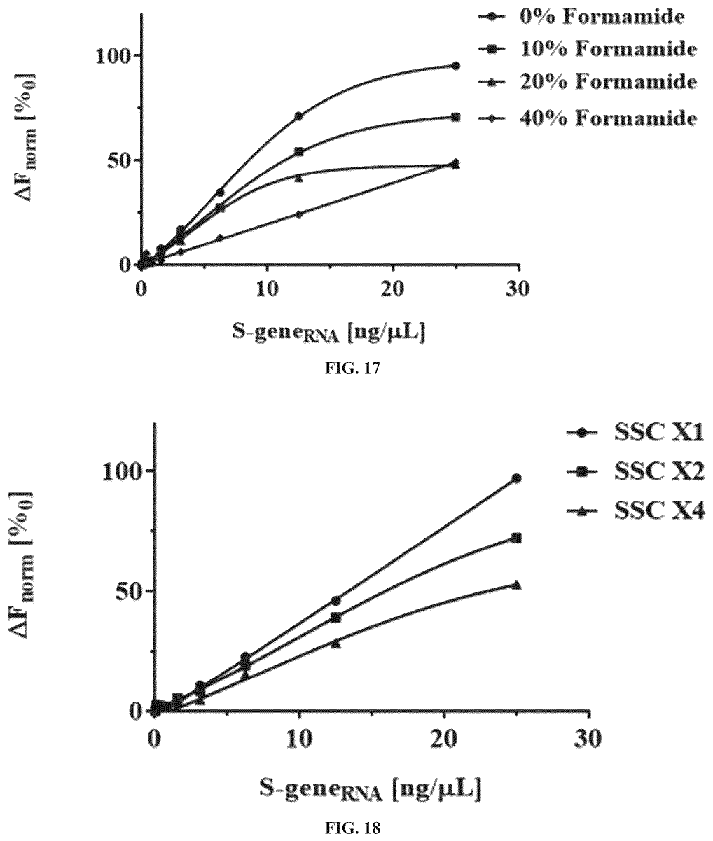

FIGS. 17-29B, 58A-58B and 63 show the effect of probe-target nucleic acid hybridization conditions on detection sensitivity. FIGS. 55-56B show that longer target nucleic acids may be associated with greater sensitivity.

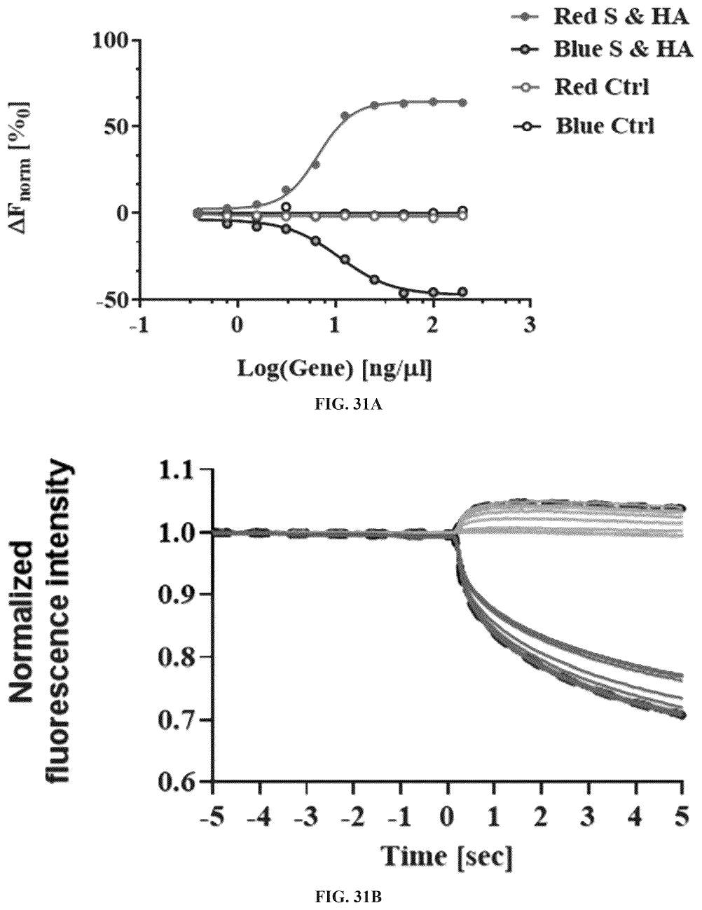

FIGS. 31A-35 show the ability to concomitantly detect two different types of RNA using two different specific probes.

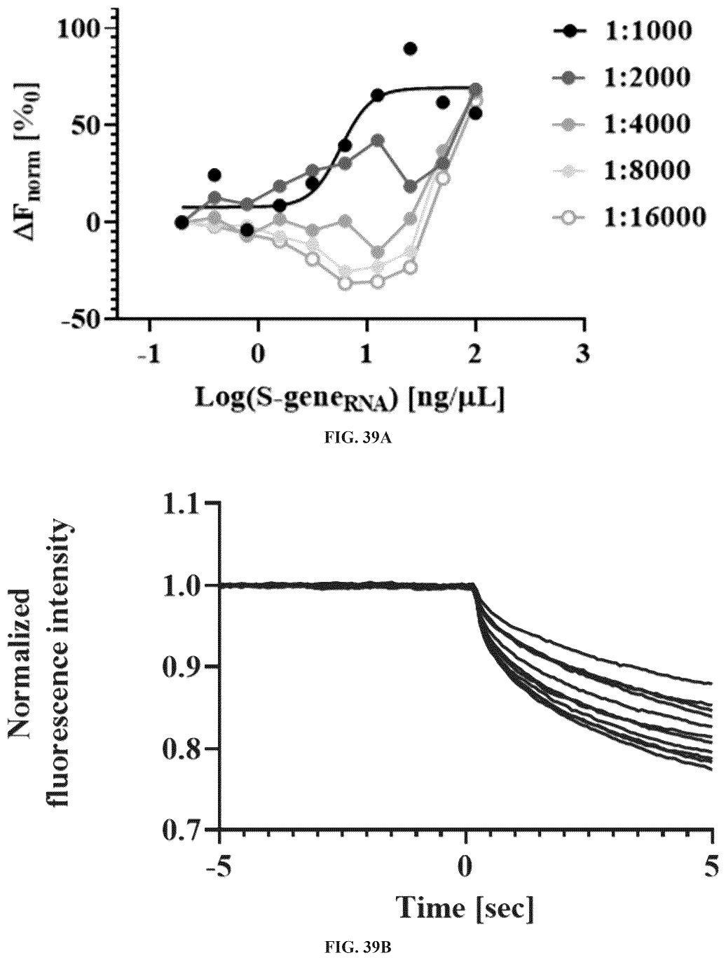

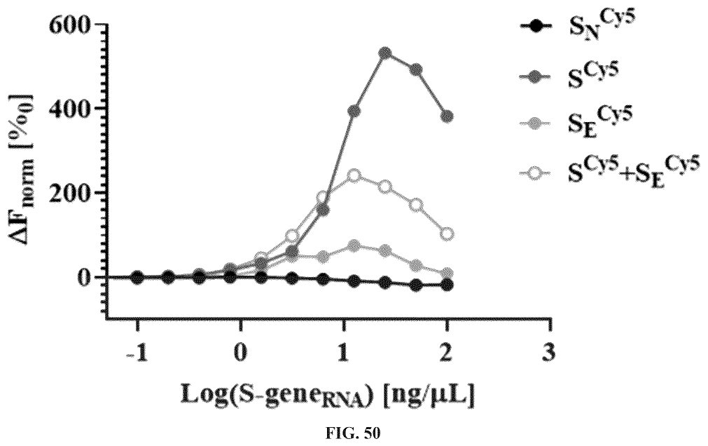

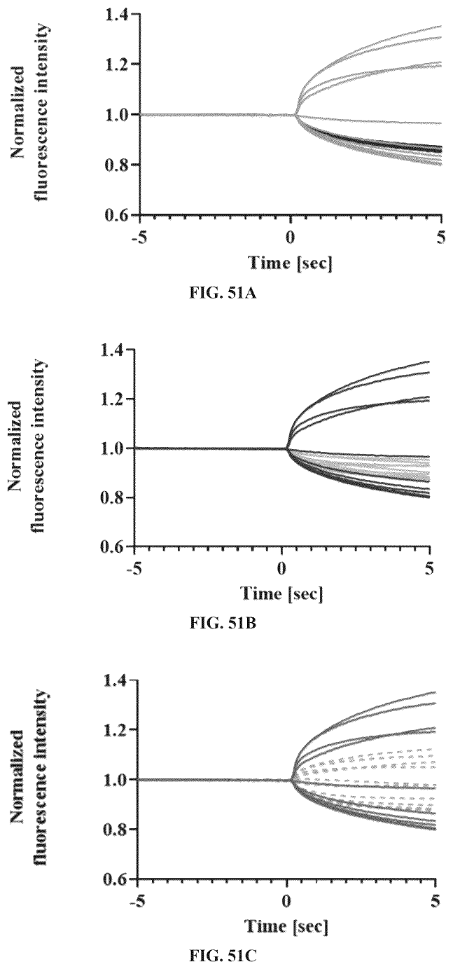

FIGS. 36-38B show the ability to enhance sensitivity by using multiple probes specific to different portions of the same target nucleic acid. FIGS. 39A-49B and 61-62B show the ability to enhance sensitivity by using a specific probe in combination with an intercalating agent. FIGS. 50-54B show the ability to enhance sensitivity by using multiple specific probes in combination with an intercalating agent.

According to an aspect of some embodiments of the invention, there is provided a method of determining a presence of a target nucleic acid in a sample. The method according to this aspect comprises:
(a) contacting a nucleic acid-containing fraction of a sample with a probe compound capable of binding to the target nucleic acid;
(b) exposing the probe compound to a temperature gradient; and
(c) detecting a signal of the probe compound during the abovementioned exposure to a temperature gradient.

The detected signal is indicative of a presence or absence of the target nucleic acid in the sample.

As exemplified herein and discussed in more detail below, the method may also be adapted so as to be suitable for simultaneously determining a presence of more than one target nucleic acid in a sample.

Herein, the term “nucleic acid-containing fraction” refers to a fraction of a sample which is prepared in such a manner as to contain (and optionally be enriched in) nucleic acids originating from the sample. It is to be appreciated that the nucleic acid-containing fraction may optionally be devoid of nucleic acids, for example, if the sample is devoid of nucleic acid.

A nucleic acid-containing fraction may be obtained according to any suitable technique known in the art (e.g., following lysis of cells from a sample); for example, such as used for RT-PCR methods. Automatic machinery for nucleic acid (e.g., RNA) extraction is common in diagnostic laboratories, and may be readily utilized for embodiments of the invention.

For example, a nucleic acid-containing fraction may be obtained by extracting a sample using guanidinium thiocyanate and water-saturated phenol and chloroform, whereby nucleic acid is recovered from the aqueous phase and typically precipitated with isopropanol. A pH of about 4-6 may be used to obtain RNA alone, whereas a pH of about 7-8 may be used to obtain DNA (e.g., in combination with RNA).

A nucleic acid-containing fraction may optionally be prepared from any type of sample in which a target nucleic acid may be present, including, without limitation, a food or water sample (e.g., for evaluating a presence of a microbe or virus in the food or water), an agricultural sample (e.g., for evaluating a presence of a pathogen of a plant or animal), or a biological sample (e.g., a biological sample obtained from a human or non-human subject, wherein the method is for determining a presence in the subject of the target nucleic acid and/or of a disease or disorder associated with the target nucleic acid).

According to an aspect of some embodiments of the invention, there is provided a method of determining a presence of one or more target nucleic acids in a human or non-human subject, the method comprising determining a presence of the target nucleic acid(s), according to any of the respective embodiments described herein, in a biological sample obtained from the subject.

Examples of biological samples include, without limitation, body fluids such as whole blood, serum, plasma, cerebrospinal fluid, urine, lymph fluids, and various external secretions of the respiratory, intestinal and genitourinary tracts, tears, saliva, milk as well as white blood cells, malignant tissues, amniotic fluid and chorionic villi.

### Target Nucleic Acid:

Herein throughout, the term “nucleic acid” refers to a polymer composed of monomers (referred to as “nucleotides”) which comprise a saccharide (typically a 5-carbon saccharide), a phosphate group and a nitrogen-containing base (typically a purine or pyrimidine derivative). Polymer chains of the nucleic acid, referred to as “strands”, may exist alone (i.e., as single-stranded nucleic acid), or in association with one or more other strands, to form double-stranded nucleic acid, triple-stranded nucleic acid, etc. “RNA” refers herein to a nucleic acid (as defined herein) wherein the saccharide of a majority of the nucleotides is ribose; and “DNA” refers herein to a nucleic acid (as defined herein) wherein the saccharide of a majority of the nucleotides is deoxyribose.

According to some of any of the embodiments described herein relating to a target nucleic acid (e.g., whose presence is determined according to a method according to any of the respective embodiments described herein), the target nucleic acid is a single-stranded nucleic acid. The single-stranded nucleic acid may optionally be a naturally occurring (e.g., in an organism) single-stranded nucleic acid. Alternatively, or additionally, the single-stranded nucleic acid may optionally be formed from a nucleic acid (e.g., a naturally occurring nucleic acid) which is not single-stranded (e.g., a double-stranded nucleic acid), for example, by heat treatment (also referred to in the art as “melting” of a nucleic acid) and/or by exposure to a suitable chemical agent. Examples of chemical agents known to produce single-stranded nucleic acid include, without limitation, formamide, guanidine, salicylate, dimethyl sulfoxide, propylene glycol and urea.

The target nucleic acid may be, for example, DNA or RNA (e.g., single stranded or double-stranded) or a copolymer thereof (wherein DNA nucleotides and RNA nucleotides are present in the same strand) or a hybrid thereof (e.g., wherein a strand of DNA is non-covalently attached to a strand of RNA). In exemplary embodiments, the target nucleic acid comprises RNA (e.g., single-stranded RNA).

In some embodiments of the present invention, target nucleic acid comprises a nucleic acid of a pathogen. In the context of the present invention, the term “pathogen” is used to describe an infectious microorganism or agent, such as a virus, a bacterium, yeast, a protozoan, a prion, a viroid, or a fungus. Since the most common use of the term “pathogen” refers to the cause of sickness, it is noted that in the context of the present invention, the term “pathogen” is meant to encompass embodiments pertaining to cancer. In the context of cancer and the present invention, the pathogen may be a gene with a carcinogenic mutation, or a gene of a mutagen; in the context of a faulty gene, the pathogen has human origins (e.g., the TP53 gene in humans), and the target nucleic acid is that of the subject rather than an external (non-self) source (e.g., human papillomavirus, or HPV).

The target nucleic acid according to any of the embodiments described herein may optionally be associated with a cancer (e.g., in which a presence of the target nucleic acid or an absence of the nucleic acid is associated with the presence of cancer), a genetic abnormality (e.g., in which a presence of the target nucleic acid or an absence of the nucleic acid is associated with the presence of the genetic abnormality), a microbe and/or a virus.

The target nucleic acid may optionally be a genomic nucleic acid or mRNA (optionally genomic nucleic acid or mRNA of a microbe and/or virus), for example, mRNA associated with transcription of a particular functional protein (e.g., a functional protein whose activity is associated with presence or absence of a cancer, a functional protein associated with absence of a genetic abnormality, or a functional protein associated with resistant to a therapeutic agent such as an anticancer agent or an antimicrobial or antiviral agent) or dysfunctional protein (e.g., a dysfunctional protein associated with a cancer or genetic abnormality). In any of the embodiments wherein the target nucleic acid is associated with a virus, the target nucleic acid may optionally be a genomic nucleic acid of a virus (e.g., an RNA genome of an RNA virus or a DNA genome of a DNA virus) and/or viral mRNA.

Herein, the term “microbe” refers to any microscopic organism, which may exist in a single-celled form and/or as a colony of cells. Examples of microbes include, without limitation, bacteria (including Gram-positive bacteria, Gram-negative bacteria, archaea and mycoplasma), protozoa and some fungi and plants.

Herein, the term “virus” refers to an agent that replicates only inside living cells of an organism, and encompasses agents composed solely of a nucleic acid, such as viroids. Examples of viruses include, without limitation, double strand DNA viruses, such as adenoviruses, herpesviruses (e.g., varicella zoster virus, herpes simplex virus-1 and/or herpes simplex virus-2), polyomaviruses (e.g., JC virus), and poxviruses; single strand DNA viruses, such as parvoviruses; double strand RNA viruses, such as reoviruses; (+)-single strand RNA viruses, such as coronaviruses (e.g., coronavirus HKU1, coronavirus NL63, coronavirus 229E, coronavirus OC43, Middle East respiratory syndrome coronavirus (MERS-CoV) and/or SARS-CoV), flaviviruses (e.g., hepatitis C virus and/or West Nile virus), hepeviruses (e.g., hepatitis E virus), picornaviruses (e.g., hepatitis A virus, enteroviruses and/or rhinoviruses, such as human enteroviruses and/or rhinoviruses) and togaviruses; (−)-single strand RNA viruses, such as orthomyxoviruses (e.g., influenza A virus, such as H1 (e.g., H1-2009) and H3 subtypes, and/or influenza B virus), filoviruses (e.g., Ebola virus), paramyxoviruses (e.g., parainfluenza virus type 1, 2, 3 and/or 4), pneumoviruses (e.g., respiratory syncytial virus and/or human metapneumovirus) and rhabdoviruses; RNA retroviruses; DNA retroviruses, such as hepadnaviruses (e.g., hepatitis B virus); satellite viruses, such as deltaviruses (e.g., hepatitis D virus); and viroids.

A microbe or virus, according to any of the respective embodiments described herein may optionally be a pathogen (e.g., of a plant, a human and/or a non-human animal) and/or associated with spoilage (e.g., of food). Examples of pathogens which are considered to be of particular medical importance include, without limitation, Enterococcus faecium, Staphylococcus aureus, Klebsiella pneumoniae, Acinetobacter baumannii, Pseudomonas aeruginosa, Enterobacter sp., and Escherichia coli.

Examples of Gram-positive bacterial pathogens include, without limitation, Enterococcus (e.g., Enterococcus faecalis and/or Enterococcus faecium), Listeria (e.g., Listeria monocytogenes), Staphylococcus (e.g., Staphylococcus aureus, Staphylococcus epidermidis, and/or Staphylococcus lugdunensis) and Streptococcus (e.g., Streptococcus agalactiae, Streptococcus pyogenes, and/or Streptococcus pneumoniae).

Examples of Gram-negative bacterial pathogens include, without limitation, Enterobacterales bacteria, such as Enterobacter (e.g., Enterobacter cloacae and Enterobacter cloacae complex members), Klebsiella (e.g., Klebsiella aerogenes, Klebsiella oxytoca and/or Klebsiella pneumonia group members), Proteus, Salmonella, and/or Serratia marcescens; Acinetobacter (e.g., Acinetobacter calcoaceticus-baumannii complex members); Bacteroides fragilis; Bordetella pertussis; Chlamydia and Chlamydophila (e.g., Chlamydophila pneumoniae); Haemophilus, influenza; Neisseria meningitidis; Pseudomonas aeruginosa; and Stenotrophomonas maltophilia.

Examples of fungal microbial pathogens include, without limitation, Candida (e.g., Candida albicans, Candida auris, Candida glabrata, Candida krusei, Candida parapsilosis, and/or Candida tropicalis) and Cryptococcus (e.g., Cryptococcus gattii and/or Cryptococcus neoformans).

In some of any of the embodiments described herein, the target nucleic acid is a ribosomal subunit (rRNA), for example, a prokaryotic ribosomal subunit (e.g., a bacterial rRNA). Examples of such rRNA include, without limitation, 16S and 23S rRNA.

In some of any of the embodiments described herein, the target nucleic acid is associated with resistance of a microbe to an antimicrobial agent (e.g., a bacterial protein associated with resistance to an antibiotic agent) or of a virus to an antiviral agent. The nucleic acid may optionally be associated with genes associated with resistance due to beta lactamases (e.g., carbapenemase (including IMP, KPC, VIM, OXA-48-like, and NDM), ESBL (extended spectrum beta lactamase), and/or CTX-M beta lactamase), colistin resistance (e.g., mcr-1), methicillin resistance (e.g., mecA/mecC and/or MREJ), and/or vancomycin resistance (e.g., Van-A or Van-B).

The skilled person will know to select a type of biological sample suitable for determining a presence of a given nucleic acid. For example, cerebrospinal fluid is a suitable sample for determining a presence of varicella zoster virus, enteroviruses, herpes simplex virus-1, herpes simplex virus-1, JC virus and/or West Nile virus.

In some of any of the respective embodiments described herein, the method comprises concomitantly determining a presence of two or more distinct nucleic acids using two or more distinct probe compounds, for example, determining a presence of a first target nucleic acid in the sample using a first probe compound and a presence of a second target nucleic acid in the sample using a second probe compound. In some such embodiments, the method comprises concomitantly detecting the signal of the first probe compound and the signal of the second probe compound. For example, the signals of the probes may optionally be fluorescent signals detected at different wavelengths (wherein excitation of the different probes may be at the same wavelength or at different wavelengths). Fluorescent labels, techniques and apparatuses suitable for concomitantly detecting signals of different wavelengths will be known to the skilled person.

As exemplified herein, determining a presence of two or more distinct nucleic acids may be useful for diagnosing (e.g., concomitantly) a presence or absence of different conditions (e.g., infections) with related symptoms, such as COVID-19 and influenza, which are each associated with symptoms such as cough, sore throat, and shortness of breath.

As further exemplified herein, determining a presence of two or more distinct nucleic acids may be useful for providing a concentration control, wherein one (or more) of the target nucleic acids is selected as having relatively constant concentrations (e.g., a human nucleic acid such as a ribosomal RNA).

Alternatively or additionally, determining a presence of two or more distinct nucleic acids may be useful for determining a presence or absence of a variant (e.g., mutant). For example, in some such embodiments, one of the nucleic acids may be unaffected by the variance (e.g., a canonical sequence of a pathogen), such that a signal associated with a probe targeting the nucleic acid is sensitive to the presence of the nucleic acid but relatively insensitive to what variant is present; and another nucleic acid represents a location of variance (e.g., point mutation), such that a signal associated with a probe targeting this nucleic acid is sensitive to whether a variant is present. A sequence variant (which may be known or unknown) may optionally be associated with a mismatch which reduces a signal associated with the probe relative to a signal in the presence of a “normal” sequence which the probe is designed for. Alternatively, a probe may optionally be designed for detection of a specific (known) sequence variant. The two nucleic acid sequences targeted by the two probes may be separate or attached, e.g., representing different portions of a single nucleic acid.

It is expected that during the life of a patent maturing from this application many relevant target nucleic acids and/or associations thereof with a disease or disorder will be uncovered and the scope of the term “target nucleic acid” is intended to include all such nucleic acids a priori.

### Probe Compound:

In some of any of the respective embodiments described herein, the probe compound comprises a nucleic acid (as defined herein) having a sequence (referred to herein interchangeably as the “probe compound sequence” or “probe sequence”) complementary to at least a portion of the target nucleic acid. In some such embodiments, the nucleic acid comprised by the probe compound comprises DNA.

Herein and in the art, a nucleic acid sequence is considered “complementary” to another sequence when parallel alignment of the two sequences (in opposite directions, that is, wherein one sequence is in a 5-prime to 3-prime direction and the other sequence is in a 3-prime to 5-prime direction) results in each base of a sequence being adjacent to an opposite base of the other sequence; wherein adenine (A) is the opposite base of thymine (T) or uracil (U), and vice versa, and guanine (G) is the opposite base of cytosine (C), and vice versa. In some of any of the respective embodiments described herein, a length of the nucleic acid (as determined according to number of nucleotides) comprised by the probe compound is no more than 10% of the length of the target nucleic acid (as determined according to number of nucleotides). In some embodiments, the length of the nucleic acid comprised by the probe compound is no more than 5% of the length of the target nucleic acid. In some embodiments, the length of the nucleic acid comprised by the probe compound is no more than 2% of the length of the target nucleic acid. In some embodiments, the length of the nucleic acid comprised by the probe compound is no more than 1% of the length of the target nucleic acid. In some embodiments, the length of the nucleic acid comprised by the probe compound is no more than 0.5% of the length of the target nucleic acid. In some embodiments, the length of the nucleic acid comprised by the probe compound is no more than 0.2% of the length of the target nucleic acid.

Without being bound by any particular theory, it is believed that when the target nucleic acid is considerably longer (e.g., at least 10-fold or at least 100-fold) than the probe compound sequence, the effect (upon thermophoresis) of binding of the target nucleic acid to the probe compound is more prominent, thereby providing greater signal resolution.

Furthermore, when the target nucleic acid is considerably longer than the probe compound sequence, a wide variety of portions of the target nucleic acid may optionally be selected as being complementary to the probe compound sequence. Thus, the probe compound sequence may optionally be selected to exhibit one or more desired characteristics, such as a relatively high GC %, absence of self-annealing or inter-loops, and/or minimization to homology to other nucleic acids, according to any of the respective embodiments described herein.

In some of any of the respective embodiments described herein, a GC % of the probe compound sequence is at least 54%. In some such embodiments, the GC % of the probe compound sequence is at least 56%. In some embodiments, the GC % of the probe compound sequence is at least 58%. In some embodiments, the GC % of the probe compound sequence is at least 60%. In some embodiments, the GC % of the probe compound sequence is at least 62%. In some embodiments, the GC % of the probe compound sequence is at least 64%. In some embodiments, the GC % of the probe compound sequence is at least 66%. In some embodiments, the GC % of the probe compound sequence is at least 68%. In some embodiments, the GC % of the probe compound sequence is at least 70%.

In some of any of the respective embodiments described herein, a GC % of the probe compound sequence is at least 90% of the highest possible GC % for a sequence of the length of the probe compound sequence. Thus, for example, if the probe compound sequence is 25 nucleotides in length, and the highest GC % for any 25-nucleotide portion of the target nucleic acid is 80% (20 of 25 nucleotides), then at least 90% of the highest possible GC % for a sequence of such a length would be a GC % of at least 72% (18 of 25 nucleotides). In some such embodiments, a GC % of the probe compound sequence is at least 95% of the highest possible GC % for a sequence of the length of the probe compound sequence. In some embodiments, a GC % of the probe compound sequence is the highest possible GC % for a sequence of the length of the probe compound sequence.

Herein, the term “GC %” refers to the percentage of nucleotides which comprise either guanine (or a guanine derivative which selectively binds non-covalently to cysteine similarly to the manner in which guanine does) or cysteine (or a cysteine derivative which selectively binds non-covalently to guanine similarly to the manner in which cysteine does).

In some of any of the respective embodiments described herein, the sequence complementary to at least a portion of the target nucleic acid is at least 14 bases in length (e.g., from 14 to 100 or from 14 to 50 bases in length). In some such embodiments, a GC % of the probe compound sequence is at least 90% of the highest possible GC % for a sequence of the length of the probe compound sequence (e.g., according to any of the respective embodiments described herein). In some of any of the aforementioned embodiments, a length of the nucleic acid comprised by the probe compound is no more than 10% or no more than 5% or no more than 2% or no more than 1% or no more than 0.5% or no more than 0.2% of the length of the target nucleic acid sequence (e.g., according to any of the respective embodiments described herein).

Without being bound by any particular theory, it is believed that sequences of at least 14 bases in length are advantageous in that the melting temperature of such sequences is determined by the GC % (as opposed to other factors), and is thus readily controllable by controlling the GC % (e.g., according to any of the respective embodiments described herein).

In some of any of the respective embodiments described herein, the sequence complementary to at least a portion of the target nucleic acid is at least 16 bases in length (e.g., from 16 to 100 or from 16 to 50 bases in length). In some such embodiments, a GC % of the probe compound sequence is at least 90% of the highest possible GC % for a sequence of the length of the probe compound sequence (e.g., according to any of the respective embodiments described herein). In some of any of the aforementioned embodiments, a length of the nucleic acid comprised by the probe compound is no more than 10% or no more than 5% or no more than 2% or no more than 1% or no more than 0.5% or no more than 0.2% of the length of the target nucleic acid sequence (e.g., according to any of the respective embodiments described herein).

In some of any of the respective embodiments described herein, the sequence complementary to at least a portion of the target nucleic acid is at least 20 bases in length (e.g., from 20 to 100 or from 20 to 50 bases in length). In some such embodiments, a GC % of the probe compound sequence is at least 90% of the highest possible GC % for a sequence of the length of the probe compound sequence (e.g., according to any of the respective embodiments described herein). In some of any of the aforementioned embodiments, a length of the nucleic acid comprised by the probe compound is no more than 10% or no more than 5% or no more than 2% or no more than 1% or no more than 0.5% or no more than 0.2% of the length of the target nucleic acid sequence (e.g., according to any of the respective embodiments described herein).

In some of any of the respective embodiments described herein, the sequence complementary to at least a portion of the target nucleic acid is at least 25 bases in length (e.g., from 25 to 100 or from 25 to 50 bases in length). In some such embodiments, a GC % of the probe compound sequence is at least 90% of the highest possible GC % for a sequence of the length of the probe compound sequence (e.g., according to any of the respective embodiments described herein). In some of any of the aforementioned embodiments, a length of the nucleic acid comprised by the probe compound is no more than 10% or no more than 5% or no more than 2% or no more than 1% or no more than 0.5% or no more than 0.2% of the length of the target nucleic acid sequence (e.g., according to any of the respective embodiments described herein).

In some of any of the respective embodiments described herein, the sequence complementary to at least a portion of the target nucleic acid is at least 30 bases in length (e.g., from 30 to 100 or from 30 to 50 bases in length). In some such embodiments, a GC % of the probe compound sequence is at least 90% of the highest possible GC % for a sequence of the length of the probe compound sequence (e.g., according to any of the respective embodiments described herein). In some of any of the aforementioned embodiments, a length of the nucleic acid comprised by the probe compound is no more than 10% or no more than 5% or no more than 2% or no more than 1% or no more than 0.5% or no more than 0.2% of the length of the target nucleic acid sequence (e.g., according to any of the respective embodiments described herein).

In some of any of the respective embodiments, the nucleic acid of the probe does not exhibit self-annealing and/or inter-loops.

Self-annealing and/or inter-loops may be determined using the OligoCalc oligonucleotide properties calculator (e.g., online at biotools(dot)nubic(dot)northwestern(dot)edu), wherein self-annealing is considered to require at least 5 self-complementary base pairs for self-dimerization, and inter-loops are considered to require at least 4 self-complementary base pairs for hairpin formation.

In some of any of the respective embodiments, the nucleic acid of the probe is selected to minimize homology with viral RNA, bacterial RNA, and/or the human transcriptome.

Homology may optionally be determined by aligning candidate sequences using NCBI BLAST algorithm versus standard database (e.g., to determine homology with viral and/or bacterial RNA) and/or human transcriptome database (e.g., to determine homology with the human transcriptome). In some embodiment, minimizing homology comprises selecting a sequence with the lowest percentage of identity with a sequence in the relevant database(s). In some embodiment, minimizing homology comprises selecting a sequence with a percentage of identity with a sequence in the relevant database(s) which is below a predetermined threshold (e.g., removing candidates with a sequence identity above the threshold). Such a threshold may optionally be in a range of from 40% to 80%, or from 50% to 70%, 60% is an exemplary threshold.

As exemplified herein, a probe compound complementary to a portion of the target nucleic acid at or close to the middle of the target nucleic acid may result in more sensitivity than a probe compound complementary to an end of the target nucleic acid.

In some embodiments, a distance between a portion of the target nucleic acid complementary to a probe compound nucleic acid and a terminus of the target nucleic acid (i.e., the distance between the terminus and the closest nucleotide of the complementary portion) is at least 10 bases, optionally at least 30 bases, optionally at least 100 bases, optionally at least 300 bases, and optionally at least 1000 bases.

In some embodiments, a distance between a portion of the target nucleic acid complementary to a probe compound nucleic acid and a terminus of the target nucleic acid is at least 1% of a length of the target nucleic acid, optionally at least 3% of a length of the target nucleic acid, optionally at least 10% of a length of the target nucleic acid, and optionally at least 30% of a length of the target nucleic acid.

The probe compound optionally comprises a label suitable for being detected according to any suitable detection technique known in the art, for example, a label conjugated to a moiety capable of binding to the target nucleic acid. Examples of suitable labels include, without limitation, a radioactive label, a fluorescent label, a phosphorescent label, and a chemiluminescent chemical.

It is expected that during the life of a patent maturing from this application many relevant labels will be developed and the scope of the term “label” is intended to include all such new technologies a priori.

In some such embodiments, the probe compound comprises a fluorescent label, i.e. the label comprises a fluorophore. Examples of suitable fluorophores include, but are not limited to, cyanine dyes (e.g., cyanine 5 dyes), fluoresceins, coumarins, carbopyronins, phycoerythrins and phycoerythrin-cyanine conjugates, rhodamine dyes (e.g., ATTO 488, Texas Red), acridine and phenothiazine dyes, green fluorescent protein, blue fluorescent protein, and the like. In exemplary embodiments, the fluorophore is a cyanine dye (e.g., cyanine 5 dye, such as described herein) and/or an ATTO 488 dye.

Additional guidance regarding fluorophore selection and/or methods of linking fluorophores to various types of molecules may be found, for example, in Richard P. Haugland, “Molecular Probes: Handbook of Fluorescent Probes and Research Chemicals 1992-1994”, 5th ed., Molecular Probes, Inc. (1994); Hermanson, “Bioconjugate Techniques”, Academic Press New York, N.Y. (1995); Kay et al. [Biochemistry 1995, 34:293]; Stubbs et al. [Biochemistry 1996, 35:937]; Gakamsky et al., “Evaluating Receptor Stoichiometry by Fluorescence Resonance Energy Transfer,” in “Receptors: A Practical Approach,” 2nd ed., Stanford C. and Horton R. (eds.), Oxford University Press, U K. (2001); and U.S. Pat. Nos. 6,037,137 and 6,350,466.

The mode by which the probe compound binds to the target nucleic acid may affect thermophoretic migration of the hybrid. Thus, the method provided herein can be enhanced by using more than one probe compound for detecting a target nucleic acid in a sample, wherein each of the probe compound is designed to bind to the target nucleic acid. This concept is demonstrated in Examples 4 and 6 hereinbelow.

Without being bound by any particular theory, it is believed that certain fluorescent labels provide an advantageous combination of relatively high spatial resolution (e.g., for selectively differentiating between adjacent regions of a temperature gradient), rapid detection, and relatively high signal-to-noise ratios at low concentrations. When using more than one probe compound in a single assay, the fluorescent labels that are attached to the nucleic acid portion of the probe compounds must differ in the color of the light they emit. While reducing the present invention to practice, it was discovered that when the fluorescent labels differ in other properties, such as charge, heat capacity, dynamic molecular volume, size/weight, flexibility, and other thermodynamic properties.

In some of any of the embodiments described herein, the probe compound comprises a nucleic acid (e.g., the moiety capable of binding to the target nucleic acid is a complementary nucleic acid) and a detectable label (e.g., fluorescent label) conjugated to a 5-prime end of the nucleic acid of the probe compound.

Without being bound by any particular theory, it is believed that conjugation of the detectable label to the 5-prime end may result in less interference with binding of the probe compound to the target nucleic acid, as compared to conjugation to another portion of the nucleic acid of the probe compound.

Without being bound by any particular theory, it is believed that thermophoretic migration of probe compounds comprising a nucleic acid and a label such as a fluorescent label is generally affected by the label to a greater degree than by the particular sequence of the nucleic acid.

### Experimental Conditions:

Contacting a nucleic acid-containing fraction of a sample with a probe compound according to any of the respective embodiments described herein may optionally be effected under conditions (e.g., temperature, pH and/or reaction mixture composition) selected to enhance binding of the probe compound to the target nucleic acid and/or reduce non-specific binding of the probe compound (which may result in a false positive signal), e.g., as exemplified in the Examples section herein.

In some of any of the respective embodiments, contacting a nucleic acid-containing fraction of a sample with a probe compound is effected (e.g., in an aqueous solution) in a presence of one or more additional compounds, for example, a salt (e.g., NaCl), a buffer (e.g., a citrate salt such as trisodium citrate), formamide and/or a surfactant (e.g., polysorbate 20).

A suitable concentration of formamide may be, for example, from 1 to 12 weight percent, optionally from 1 to 4 weight percent, or from 2 to 8 weight percent, or from 4 to 12 weight percent.

A suitable concentration of surfactant (e.g., polysorbate 20) may be, for example, from 0.005 to 0.5 weight percent, optionally from 0.005 to 0.05 weight percent, or from 0.015 to 0.15 weight percent, or from 0.05 to 0.5 weight percent. In exemplary embodiments, the concentration is about 0.05 weight percent.

Without being bound by any particular theory, it is believed that formamide and/or surfactant may be useful for reducing non-specific binding of nucleic acid strands, for example, wherein the target nucleic acid comprises single strand RNA. It is further believed that the formamide and/or surfactant concentrations described herein will not interfere considerably with the detection of the target nucleic acid (e.g., as exemplified herein).

A suitable concentration of salt (e.g., NaCl), not including a buffer, may be, for example, in a range of from 0.15 mM to 1.5 M (e.g., from 50 mM to 1.5 M), or from 0.2 mM to 1.2 M (e.g., from 75 mM to 1.2 M), or from 0.3 mM to 1 M (e.g., from 100 mM to 1 M), or from 0.4 mM to 800 mM (e.g., from 125 mM to 800 mM), or from 0.6 mM to 600 mM (e.g., from 150 mM to 600 mM. In some exemplary embodiments, the salt (e.g., NaCl) concentration is about 150 mM.

A suitable concentration of buffer (i.e., a total concentration of all forms of the buffer compound, including all acid and base forms) may be, for example, in a range of from 5 mM to 1.5 M (e.g., from 5 to 150 mM), or from 7.5 nM to 1.2 M (e.g., from 7.5 to 120 mM), or from 10 mM to 1 M (e.g., from 10 to 100 mM), or from 12.5 mM to 800 mM (e.g., from 12.5 to 80 mM), or from 15 mM to 600 mM (e.g., from 15 to 60 mM).

Besides controlling pH, a buffer may also be used to increase the ion concentration (e.g., in addition to a salt which is not a buffer). A suitable total concentration of buffer (e.g., citrate buffer) and salt (e.g., NaCl) may be for example, from 5 mM to 3 M (e.g., from 100 mM to 3 M), or from 7.5 mM to 2.5 M (e.g., from 150 mM to 2.5 M), or from 10 mM to 2 M (e.g., from 200 mM to 2 M), or from 12.5 mM to 1.6 M (e.g., from 250 mM to 1.6 M), or from 15 mM to 1.2 M (e.g., from 300 mM to 1.2 M).

Alternatively or additionally, a concentration of salt (e.g., NaCl) and/or buffer (e.g., citrate buffer) may optionally be selected such that a total concentration of ions (in the environment in which probe compound and nucleic acid-containing fraction are contacted) is in a range of from 0.3 mM to 3 M (e.g., from 100 mM to 3 M), or from 0.4 mM to 2.5 M (e.g., from 150 mM to 2.5 M), or from 0.6 mM to 2 M (e.g., from 200 mM to 2 M), or from 0.8 mM to 1.6 M (e.g., from 250 mM to 1.6 M), or from 1.2 mM to 1.2 M (e.g., from 300 mM to 1.2 M).

Exemplary solutions of buffer and salt (for contacting a nucleic acid-containing fraction of a sample with a probe compound) include about 60 mM trisodium citrate and about 600 mM NaCl for use with a probe compound comprising an ATTO 488 dye; and about 150 mM trisodium citrate and about 150 mM NaCl for use with a probe compound comprising a cyanine dye (according to any of the respective embodiments described herein), optionally even when used together with another probe compound comprising another dye, e.g., ATTO 488 (according to any of the respective embodiments described herein).

Contacting a nucleic acid-containing fraction of a sample with a probe compound according to any of the respective embodiments described herein may optionally be effected at a pH in a range of from 5 to 9, optionally from 6 to 8. In exemplary embodiments, the pH is about 7. Various buffers suitable for obtaining such a pH will be known to the skilled person. Citrate is an exemplary buffer for obtaining such a pH.

In some of any of the respective embodiments, contacting a nucleic acid-containing fraction of a sample with a probe compound is effected (e.g., in an aqueous solution) at a temperature in a range of from 32° C. to 82° C., optionally from 42° C. to 82° C., or from 52° C. to 82° C., or from 62° C. to 82° C., or from 32° C. to 72° C., or from 42° C. to 72° C., or from 52° C. to 72° C., or from 32° C. to 62° C., or from 42° C. to 62° C.

In some of any of the respective embodiments, contacting a nucleic acid-containing fraction of a sample with a probe compound is effected (e.g., in an aqueous solution) for a duration of at least 1 minute (e.g., from 1 to 240 minutes or from 1 to 60 minutes, or from 1 to 20 minutes, or from 1 to 10 minutes), or at least 2 minutes (e.g., from 2 to 240 minutes or from 2 to 60 minutes, or from 2 to 20 minutes, or from 2 to 10 minutes), or at least 5 minutes (e.g., from 5 to 240 minutes or from 5 to 60 minutes, or from 5 to 20 minutes, or from 5 to 10 minutes), or at least 10 minutes (e.g., from 10 to 240 minutes or from 10 to 60 minutes, or from 10 to 20 minutes).

In some exemplary embodiments, contacting a nucleic acid-containing fraction of a sample with a probe compound is effected at about 62° C. for from 5 to 10 minutes.

Without being bound by any particular theory, it is believed that moderate heating, such as at a temperature in a range of from 32° C. to 82° C., according to any or the respective embodiments described herein), for a sufficient time (e.g., at least 1 minute, according to any or the respective embodiments described herein) facilitates moving the system towards thermodynamic equilibrium, whereby the probe compound becomes bound to the most energetically favorable ligand (e.g., the target nucleic acid) rather than to the most kinetically favorable ligands (which may represent non-specific binding).

In some of any of the respective embodiments, a suitable temperature and duration of contacting for a given probe compound and target nucleic acid is selected by a calibration procedure, comprising contacting the probe compound with a target nucleic acid control (e.g., using various concentrations of target nucleic acid) at different temperatures (e.g., in a range of 32° C. to 82° C., according to any of the respective embodiments described herein), optionally for about 10 minutes. Upon selecting a suitable temperature, a suitable duration is optionally selected by contacting the probe compound and target nucleic acid (e.g., using various concentrations of target nucleic acid) at the selected temperature for various durations, optionally durations of up to 240 minutes. When more than one probe compound is used (e.g., according to any of the respective embodiments described herein), such a calibration procedure may optionally be effected for each probe compound (and its respective target nucleic acid), such that a selected temperature and duration of contacting of the probe compounds and target nucleic acid(s) is suitable for all of the probe compounds being used. In general, when multiple temperatures and/or durations provide results of similar quality, the lowest temperature and shortest duration will typically be preferable.

In some of any of the respective embodiments described herein, the probe compounds are subjected to denaturing conditions prior to being contacted with a nucleic acid-containing fraction. The denaturing conditions optionally comprise a temperature of at least 80° C. (e.g., from 80 to 120° C.) or at least 90° C. (e.g., from 90 to 120° C.) or at least 100° C. (e.g., from 100 to 120° C.), for example, for a period of at least 1 minute or at least 2 minutes or at least 5 minutes; and optionally followed by cooling (e.g., at a temperature of 4° C.), for example, for a period of at least 1 minute or at least 2 minutes or at least 5 minutes. Exemplary denaturing conditions include a temperature of about 100° C. for about 5 minutes, followed by cooling at 4° C. for about 5 minutes.

The denaturing conditions may optionally be effected in a solution (e.g., comprising buffer, salt, formamide and/or surfactant) such as used for contacting a nucleic acid-containing fraction of a sample with a probe compound according to any of the respective embodiments described herein; or with a moderately more concentrated solution (e.g., concentrated ×2) such that the conditions for contacting a nucleic acid-containing fraction of a sample with a probe compound (according to any of the respective embodiments described herein) may be readily obtained by dilution.

In some of any of the respective embodiments described herein, the method further comprises contacting the nucleic acid-containing fraction of the sample with an additional compound capable of binding to nucleic acids (in addition to the probe compound according to any of the respective embodiments described herein), optionally wherein binding to nucleic acids is independent of nucleic acid sequence (e.g., as opposed to a probe compound selective for a specific sequence). Contacting with the additional compound is optionally effected subsequently to contacting with the probe compound, for example, so as to reduce interference with binding of the probe compound to the target nucleic acid, and/or facilitate interaction of the additional compound with a probe compound-target nucleic acid complex.

In some of any of the respective embodiments described herein, the additional compound comprises at least one intercalating agent. In the context of the present invention, the term “intercalating agent” refers to substances that bind between the planar bases of nucleic acids, such as the hybrids which forms from the target nucleic acid and the probe compound. Examples of intercalating agent include, without limitation, anthracyclines (e.g., doxorubicin, daunorubicin, epirubicin, and idarubicin), acridine dyes (e.g., proflavine, acridine orange, quinacrine, and substituted derivatives thereof), phenanthridine derivatives (e.g., ethidium bromide, propidium iodide), berberine, dactinomycin, thalidomide, 4′,6-diamidino-2-phenylindole (DAPI), and the commercial agent Midori™ Green. Doxorubicin is an exemplary anthracycline.

When an anthracycline is utilized, the total concentration of anthracyclines is optionally at least 25 μM, optionally at least 50 μM, and optionally at least 75 μM. In some exemplary embodiments, the concentration of anthracycline (e.g., doxorubicin) is about 100 μM. As exemplified herein, a relatively high concentrations of anthracycline (e.g., at least 50 μM, or about 100 μM) exhibited substantially different behavior than did lower concentrations (e.g., about 1-10 μM), such that signal resolution may be improved for reasons unrelated to a simple linear correlation of signal to concentration.

As exemplified herein, a suitable concentration of Midori™ Green may be obtained by diluting a stock solution thereof to a degree in a range of from 1:1000 to 1:8000.

In some of any of the respective embodiments, a suitable temperature and duration of contacting of the nucleic acid-containing fraction of the sample with a given additional compound capable of binding to nucleic acids is selected by a calibration procedure (e.g., similar to the calibration procedure described herein for probe compounds), comprising testing the additional compound at different temperatures (e.g., using various concentrations of target nucleic acid) to select a suitable temperature, and/or selecting a suitable duration by testing the additional compound (e.g., at a temperature selected as described herein) using different durations of contacting (e.g., using various concentrations of target nucleic acid).

Compounds and compositions utilized herein according to any of the respective embodiments described herein—for example, reagents for preparing a nucleic acid-containing fraction of a sample, probe compound(s), additional compound(s) capable of binding to nucleic acid, and a composition suitable for contacting the probe compound with a nucleic acid-containing fraction (e.g., comprising a buffer, a salt, formamide and/or a surfactant) or ingredients for preparing such a composition—may optionally be included in a diagnostic kit/article of manufacture, preferably along with appropriate instructions for use and labels indicating regulatory approval (e.g., FDA approval) for use in determining presence of a target nucleic acid and/or diagnosing a disease or disorder according to any of the respective embodiments described herein.

Such a kit can include, for example, at least one container including at least one probe compound (and optionally additional compound(s) capable of binding to nucleic acids) according to any of the respective embodiments described herein; ingredients for preparing a composition suitable for contacting the probe compound with a nucleic acid-containing fraction (e.g., comprising a buffer, a salt, formamide and/or a surfactant, according to any of the respective embodiments described herein); and/or at least one container including one or more reagents for preparing a nucleic acid-containing fraction of a sample. The kit may optionally further comprise one or more additional compound(s) capable of binding to nucleic acids in a separate container from the probe compound(s), so as to allow for addition only subsequently to contact of the probe compound(s) with the nucleic acid-containing fraction (e.g., as described herein). The kit may also include appropriate buffers and preservatives for improving the shelf-life of the kit.

The ingredients for preparing a composition suitable for contacting the probe compound with a nucleic acid-containing fraction may optionally be in the same container as the probe compound(s) and/or additional compound(s) capable of binding to nucleic acid, e.g., in the form of a liquid composition (e.g., aqueous solution comprising the probe compound(s) and/or additional compound(s)) or powder which results in such a liquid composition upon addition of water. Alternatively or additionally, the ingredients of the composition suitable for contacting the probe compound with a nucleic acid-containing fraction may be divided unevenly between more than one container of the kit, such that the composition is formed upon combining the contents of the different containers.

### Temperature Gradient and Signal Detection:

Exposure of the probe compound to a temperature gradient according to any of the respective embodiments described herein is optionally effected by applying heating or cooling to a sample comprising the probe compound (wherein different portions of the sample are heated or cooled to different degrees). Heating is typically more convenient, especially for effecting rapid temperature changes. Heating may optionally be effected by electromagnetic radiation, such as infrared radiation, to a selected location. A laser (e.g., infrared laser) may be particularly convenient, for example, by being readily capable of irradiating only a small, well defined area, and only with a specific wavelength (e.g., thereby avoiding wavelengths which may interact with compounds in a manner other than generating a temperature gradient).

Upon generation of a temperature gradient by heating, a portion of the sample is heated to the maximal extent (i.e., more than any other region in the sample) and this portion is referred to herein as the “high temperature region” of the gradient; and a portion of the sample is heated to the minimal extent (i.e., less than any other region in the sample, optionally not heated at all) and this portion is referred to herein as the “low temperature region” of the gradient.

The low temperature region, according to some of any of the respective embodiments, comprises a temperature of about 25° C. (i.e., from 20 to 30° C., optionally from 23 to 27° C., and optionally 25° C.). In embodiments wherein the low temperature region does not undergo substantial heating at all, this will also be the temperature of the sample prior to heating.

The temperature gradient is optionally generated in a narrow tube (e.g., capillary) comprising a sample (e.g., nucleic acid-containing fraction) being tested, which may simplify and/or amplify the temperature gradient by essentially reducing the system to one dimension (the axis of the tube).

Herein, the phrase “detecting the signal” encompasses acquiring experimental data (for example, measuring light intensity, e.g., using a photodetector) and analysis of the data. Analysis of the data may optionally comprise, for example, one or more of (optionally all of): averaging data points acquired over a time period to obtain a single value; comparing data associated with a temperature gradient and data associated with a baseline, optionally presented as a ratio (e.g., normalization); and comparing data associated with a nucleic acid-containing fraction being tested with a control sample lacking the target nucleic acid.

Accordingly, the term “signal” may refer herein to a final product of data analysis or to raw data (e.g., detected fluorescent light intensity) or to partially processed data to be subjected to further data analysis (e.g., normalized data indicating a difference between data associated with a temperature gradient and data associated with a baseline, but which has not been compared to data for a control sample lacking the target nucleic acid).

In some of any of the respective embodiments described herein, acquiring experimental data is effected (i.e. begins) at least one second after initial exposure of the probe compound to the temperature gradient. The “pause” of at least one second may optionally be useful to allow time for molecules to react (e.g., by thermophoretic migration) to a generated temperature gradient and/or to allow time for the temperature gradient to increase (e.g., to generate a larger signal). In some embodiments, acquiring experimental data is effected at least about 10 seconds after initial exposure of the probe compound to the temperature gradient, and in some embodiments, at least about 25 seconds after initial exposure of the probe compound to the temperature gradient. Relatively long pauses (e.g., of about 25 seconds or more) before data acquisition may be useful for allowing the system to arrive at or close to a steady state, which may be associated with a maximal signal (and resolving power) and/or with a reduced level of sample-to-sample variation. The length of pause needed to arrive at or close to a steady state may be affected by the heating intensity (e.g., laser intensity), dimensions and/or heat loss of a given system, and may be readily determined for any given system.

In some of any of the respective embodiments described herein, acquiring experimental data is effected for a duration of at least one second, optionally at least 2 seconds and optionally at least 5 seconds. The duration of acquiring experimental data may be correlated with signal-to-noise resolution.

In some of any of the respective embodiments described herein, acquiring experimental data is effected (i.e., begins) no more than 60 seconds (e.g., from 1 to 60 seconds or from 10 to 60 seconds or from 25 to 60 seconds) after initial exposure of the probe compound to the temperature gradient, optionally no more than 10 seconds (e.g., from 1 to 10 seconds), no more than 5 seconds (e.g., from 1 to 5 seconds), or no more than 2 seconds (e.g., from 1 to 2 seconds) after initial exposure to the temperature gradient.

In some of any of the respective embodiments described herein, acquiring experimental data is effected for a duration of no more than 10 seconds (e.g., from 1 to 10 seconds or from 2 to 10 seconds or from 5 to 10 seconds), optionally no more than 5 seconds (e.g., from 1 to 5 seconds or from 2 to 5 seconds), optionally no more than 2 seconds (e.g., from 1 to 2 seconds), and optionally for about 1 second.

In some of any of the respective embodiments described herein, acquiring experimental data is completed no more than 60 seconds (e.g., from 2 to 60 seconds or from 10 to 60 seconds or from 25 to 60 seconds) after initial exposure of the probe compound to the temperature gradient, optionally no more than 10 seconds (e.g., from 2 to 10 seconds), or no more than 5 seconds (e.g., from 2 to 5 seconds) after initial exposure to the temperature gradient.

Relatively short pauses and/or data acquisition duration may facilitate a higher test rate which may be desirable even if it entails a moderate decrease in resolution.

Acquiring experimental data, according to any of the respective embodiments, may be effected for any one or more region, optionally at the high temperature region and/or at the low temperature region of the temperature gradient. Optionally, acquiring experimental data is effected for one region, which is the region in which temperature changes the most over time, for example, the high temperature region; and compared to a baseline data acquired prior to application of the temperature gradient, for example, wherein the acquired data for a temperature gradient is normalized to the baseline data (e.g., represented as a ratio of acquired data for a temperature gradient to baseline data) in the absence of a temperature gradient. Alternatively or additionally, acquiring experimental data is effected at two regions (optionally simultaneously) of the temperature gradient (e.g., for example, the high temperature region and low temperature region), for example, wherein the difference in data values from the two regions is normalized to the data values of either of the two regions (e.g., represented as a ratio of difference between the two values to one of the values).

In some of any of the embodiments described herein, baseline data is acquired prior to application of a temperature gradient (e.g., in addition to acquiring experimental data after exposure of the probe compound to a temperature gradient, according to any of the respective embodiments described herein), and the baseline data is optionally acquired immediately before application of the temperature gradient, for example, the acquiring data is completed no more than one second (optionally no more than 0.1 second) prior to generation of the temperature gradient. The duration of data acquisition for the baseline signal may be according to any of the embodiments regarding duration of the signal described elsewhere herein (e.g., at least one second and/or optionally no more than 10 seconds).

In some of any of the embodiments described herein, detecting the signal further comprises comparing a signal of the probe compound in the presence of a nucleic acid-containing fraction with a signal of a probe compound in the presence of a control sample in which the target nucleic acid is known to be absent, for example, determining a change in the signal with respect to the signal of the probe compound in the control, that is, a difference between the signal for the nucleic acid-containing fraction and the signal for the control sample.

Optionally, a difference between the signal for the nucleic acid-containing fraction and the signal for the control sample that is beyond a predetermined threshold of the probe compound in the absence of the target nucleic acid is indicative of a presence of the target nucleic acid in the sample. That is, a very small difference (below a predetermined threshold) of a given sample from the control sample (without the target nucleic acid) may be regarded as merely routine sample-to-sample variation which is not indicative of a presence of the target nucleic acid in the sample. The predetermined threshold may optionally be selected by making multiple determinations of control samples in order to evaluate the sample-to-sample variation, and assuming such variation is representative of the variation for future measurements. Alternatively or additionally, the threshold may be determined by estimation, based on an estimated based on experience with comparable technology. In addition, the predetermined threshold may optionally be selected according to the needs of the practitioner, e.g., based on the relative costs of a false positive and false negative error. For example, for a severe, but readily treatable disease, the cost of a false positive is relatively low as compared to the cost of a false negative, which favors a relatively low threshold; whereas the relative cost of a false positive may be higher, which favors a relatively high threshold, for research purposes and for a disease which is not particularly severe (e.g., wherein the cost of a false negative is relatively low), or which is severe but difficult to treat (e.g., having a high psychological cost if an untreatable lethal condition is falsely diagnosed and/or a high economic cost if a condition with an expensive treatment is falsely diagnosed).

The data acquisition and data analysis according to any of the respective embodiments described herein may optionally be performed using a commercially available system for performing microscale thermophoresis (MST) measurements.

In some of any of the respective embodiments described herein, the signal is a fluorescent signal, e.g., the probe compound comprises a fluorescent label and signal detection comprises exposing a region of the sample to radiation at an excitation wavelength suitable for the fluorescent label, and detecting radiation at an emission wavelength suitable for the fluorescent label.

### Diagnosis:

Medical diagnosis is the process of determining which disease or condition explains a subject's symptoms and signs, whereas the information required for diagnosis is typically collected from a history and physical examination of the subject. According to some embodiments of the present invention, at least one of the procedures effected for diagnosis includes the method(s) provided herein. According to an aspect of some embodiments of the invention, there is provided a method of diagnosing a disease or disorder in a subject associated with a nucleic acid, the method is effected by determining the presence of a target nucleic acid associated with the disease or disorder, according to any of the embodiments described herein relating to a method of determining a presence of a target nucleic acid in a sample taken from the subject. A presence of the target nucleic acid in the sample taken from the subject is indicative of a presence of the disease or disorder in the subject. Such a disease or disorder may optionally be, for example, cancer and/or genetic abnormality, or a disease or disorder associated with a pathogen (fungus, microbe or virus).

Since carcinogenesis relates to mutations and to mutagens, the present invention provides means to determine, both qualitatively and quantitatively, the presence of a mutated gene suspected of carcinogenicity, and/or a nucleic acid of a carcinogenic mutagens.

In the context of embodiments of the present invention, a disease or disorder associated with a pathogen, such as a fungal, microbial or viral infection, include chronic, subacute and/or acute infections, bacteremia and viremia. Diagnosis of one or more viral infection is a particularly useful application of some embodiments of the invention, as viruses are difficult to identify with confidence except by detection of specific nucleic acids.

In addition, such a method may optionally be for diagnosing a presence or absence of a plurality of diseases or disorders, by detecting a presence or absence of a plurality of target nucleic acids using a plurality of probe compounds each corresponding to each of the target nucleic acids, each target nucleic acid being associated with a different disease or disorder. In one embodiment, the plurality of diseases or disorders are viral infections that are associated with similar symptoms.

In some embodiments, the method provided herein is useful for diagnosing a presence or absence of a diseases or disorders having more than one indicator, wherein each indicator is associated with a different target nucleic acid. The method is effected by detecting a presence or absence of a plurality of target nucleic acids using a plurality of probe compounds, each corresponding to each of the target nucleic acids, each target nucleic acid being associated with a different indicator of the disease or disorder.

In some embodiments, the method is used for quantitative determination of a target nucleic acid in a sample. While qualitative determination of the presence of an agent causing a disease or disorder in a subject is of high medicinal value, particularly when provided rapidly, a quantitative determination of the same allows the practitioner to decide on, and follow a course of treatment and/or a regimen, while the subject is being treated and monitored, and thereby further allow the practitioner to modify the treatment and/or regimen based on the results obtained by using the method provided herein. Furthermore, rapid quantitative determination such as obtainable using the method provided herein, can be used for research as well as diagnostic purposes, as an assay for the effectiveness of a pharmaceutically active agent, a course of treatment/regimen, and the overall wellbeing of a subject, particularly when the method is useful for following more than one target nucleic acid simultaneously and concomitantly.

Herein, the terms “diagnosing”, “diagnosis” and grammatical derivatives thereof refer to determining presence or absence of a pathology (e.g., a disease, disorder, condition or syndrome), classifying a pathology or a symptom, determining a severity of the pathology, monitoring pathology progression, forecasting an outcome of a pathology and/or prospects of recovery, and/or screening of a subject for a specific disease or disorder.

Screening of the subject for a specific disease or disorder according by identifying presence of an associated target nucleic acid may optionally be followed by substantiation of the screen result using another method, for example, confirming a presence of the target nucleic acid using PCR technology.

As discussed hereinabove, the target nucleic acid is optionally associated with resistance of a microbe to an antimicrobial agent (e.g., a bacterial protein associated with resistance to an antibiotic agent) or of a virus to an antiviral agent. Detecting a nucleic acid associated with resistance to an antimicrobial or antiviral agent may be useful, for example, in determining therapeutic strategy. For example, the difference between successful and unsuccessful antibiotic treatment of bacterial infections may be particularly acute, wherein a suitable antibiotic is commonly extremely effective against bacterial infection, whereas an uncontrolled bacterial infection (e.g., due to use of an antibiotic to which the bacterium is resistant) may result in a serious medical crisis and even death, and/or to infection of additional individuals.

Without being bound by any particular theory, it is believed that detecting a target nucleic acid associated with resistance to an antimicrobial or antiviral agent according to respective embodiments of the invention can avoid the lengthy need to monitor growth of the microbe or virus in the presence of the antimicrobial or antiviral agent in order to assess susceptibility; as well as provide rapid identification of the pathogen. For example, current techniques may frequently require 24-48 hours to isolate and identify a bacterium associated with an infection, followed by an additional 24-48 hours to determine an antibiotic resistance profile in order to decide on a therapeutic strategy.

In contrast, current gold standard methodologies such as rapid PCR tests and mass-spectrometry for protein pattern recognition require bacterial isolation process prior to measurements and do not provide data about bacterial growth and active antibiotic resistance genes.

Cancers which may be associated with a target nucleic acid and/or diagnosed according to any of the respective embodiments described herein can be any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute—megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

### Additional Definitions

As used herein the term “about” refers to ±10%, and in optional embodiments ±5%. In the context of a temperature, the term “about” refers to ±5° C.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

### Materials:

DNA probes conjugated to Cyanine 5 (Cy5) or ATTO 488 dye were obtained from Metabion (Germany).

The structures of the dyes used in this study are presented below:

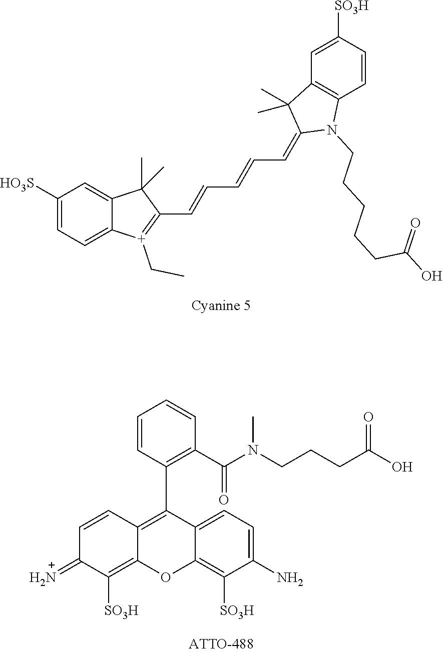

Dulbecco's modified Eagle's medium (DMEM) was obtained from Gibco.

Doxorubicin (hydrochloride) was obtained from Tzamal D-Chem Laboratories (Israel).

Fetal bovine serum was obtained from Biological Industries (Israel).

Penicillin-streptomycin was obtained from Biological Industries (Israel).

Phosphate buffered saline (PBS) buffer with 500 mM phosphate and 1500 mM NaCl (referred to herein as “X10”) was obtained from Biological Industries (Israel). Other PBS concentrations were obtained by dilution.

Saline sodium citrate (SSC) buffer with 60 mM trisodium citrate and 600 mM NaCl (referred to herein as “X4”), pH 7, was obtained from Bio-Lab Ltd. (Israel). Other buffer concentrations (e.g., X2 or X1) were obtained by dilution.

Tris was obtained from Sigma. Tris/NaCl buffer was prepared by dissolving Tris and NaCl in water at the indicated concentrations.

TRIzol™ reagent was obtained from Invitrogen.

TWEEN™ 20 (polysorbate 20) surfactant was obtained from Bio-Lab Ltd. (Israel).

### Cell Culture:

U2 osteosarcoma (OS) cells stably expressing inducible YFP-24×MS2 were maintained in low glucose Dulbecco's modified Eagle's medium (DMEM) containing 10% fetal bovine serum (FBS) and penicillin-streptomycin. Transcription was induced with 1 μg/ml doxycycline overnight and after, cells were harvested on cold ice with PBS X1. RNA was extracted from the cell pellet as described herein.

### Cloning:

In order to produce RNA of desired viral genes, desired DNA templates were cloned into a pcDNA3.1+ vector, which contains a T7 promoter that is required for in-vitro transcription using a T7 in-vitro transcription kit (Promega).

2019 nCoV (SARS CoV2) whole genome sequence was obtained from NCBI (GenBank: MN908947). Surface glycoprotein (gene S) was selected for the detection of the coronavirus, and the probes had been designed to match a SARS-CoV2-specific segment of the gene reviewed by Katano & Suzuki [“Detection of 2019-novel coronavirus sequence from clinical specimen”, available at www(dot)niid(dot)go(dot)jp/niid/images/pathol/pdf/Detection_of_nCoV_report200121.pdf]. A 1091 bp sequence from this region (1379-2468) was analyzed by NEBcutter™ program to find restriction enzymes with 0 cuts, and BamHI and EcoRI restriction sites were added to the 5′ and 3′ ends of the segment, respectively. In addition, 5 more nucleotides were added at each side in order to allow an efficient restriction, as recommended by New England BioLabs [“Cleavage Close to the End of DNA fragment” at international(dot)neb(dot)com]. The final sequence was obtained as a gBlock™ gene fragment from Integrated DNA Technologies (1 g yield). In order to form BamHI and EcoRI sticky ends, the insert was incubated for 1 h with 1 μl BamHI and 1 μl EcoRI-HF (20,000 units/ml) in CutSmart® buffer (New England BioLabs). The cleaved product was cleaned using a gel and PCR cleanup kit (Promega). In parallel, pcDNA3.1+ vector was restricted as described, and the cleaved product was isolated in 1% agarose gel. The relevant band had been cut from the gel and cleaned using a gel and PCR cleanup kit (Promega). Ligation was performed using Biogase™ fast ligation kit (Bio-Lab, Israel) following the manufacturer's protocol. Ligation products were transformed into DH5α bacteria, as follows. A total volume of the ligation reaction was added to 100 μl competent cells; followed by 30 minute incubation on ice, 2 minute heat shock at 42° C., 1 minute on ice, and addition of 1 ml LB (lysogeny broth). Recovery was then performed at 37° C. for 1 hour with shaking at 650 rpm, followed by 5 minute centrifugation at 4500 rpm. The supernatant was then discarded, leaving a volume of about 100 μl, and the obtained pellet resuspended and seeded on an agar petri dish containing 100 μg/ml ampicillin.

The bacteria were then grown overnight at 37° C., and one colony was selected for further growth in liquid media. The plasmid was extracted from the bacteria using a miniprep kit (Promega), and sequenced using universal primers for pcDNA3.1+.

N1H1 hemagglutinin (HA) gene was selected from among the WHO-approved target genes for detection of influenza virus. The sequence was obtained from NCBI (GenBank: CY249803.1), and a 1036 bp segment was taken from the middle of the gene (1379-2468). The HA insert was designed with EcoRI and XhoI restriction sites on the 5′ and 3′ ends, respectively. The insert was cloned into a pcDNA3.1+ vector using procedures such as described herein for S-gene.

S-gene+HA gene hybrid artificial gene was created and cloned into pcDNA3.1+. For the cloning of HA gene to the 3′ extension of S-gene, an HA gene insert was cut from the pcDNA vector using EcoRI and XhoI as described above. In parallel, pcDNA-S gene plasmid was cut using the same enzymes. The products were then separated using 1% agarose gel. The relevant bands were cut and cleaned using a gel and PCR cleanup kit (Promega). The HA insert was cloned to the pcDNA-S vector using procedures such as described hereinabove.

### Probe Design:

Target sequences (of SARS CoV2 S-gene, N1H1 HA-gene, and human 18S rRNA) were taken from NCBI GenBank. In order to achieve maximal melting temperature (Tm), an indication of a strong binding potential, GC-rich regions were detected in each sequence, based upon which the probes were designed.

Sequences of 20-35 nucleotides in length were uploaded to an NEB Tm calculator to determine GC content and Tm. In addition, physical constants of the oligonucleotides and self-complementary potential were analyzed using the OligoCalc oligonucleotide properties calculator. Potential probe sequences with self-complementarity of a minimum of 5 bp required for self-dimerization and/or a minimum of 4 bp required for hairpin formation were filtered out.

In order to enhance specificity of the probes, the sequences were aligned in NCBI BLAST versus standard database and the human transcriptome database. Potential probe sequences with any partial match to other pathogens and/or high similarity (more than 60% identity) to a sequence in the human transcriptome were filtered out. Since the probe designed for the human 18S rRNA was intended to be used as a positive internal control in the detection, its off-targets parameters were less rigid.

The obtained probe sequences, selected for maximal Tm value and minimal self-binding and off-targets, were then converted to the reverse complement sequences using Reverse Complement (Bioinformatics).

### In-Vitro Transcription:

PcDNA3.1+ containing 1000 bp of either S-gene or HA-gene was linearized with Xho I restriction enzyme overnight and submitted to an in-vitro transcription process using a T7 RiboMAX™ kit (Promega) and cleared from DNA using DNase for 15 minutes at 37° C.

### RNA Extraction:

RNA was extracted from samples using the guanidinium thiocyanate phenol-chloroform extraction technique, using TRIzol™ reagent. 1 ml of TRIzol™ reagent was added to a cell pellet or RNA solution after in vitro transcription. The samples were then incubated for 5 minutes at room temperature. For phase separation, 0.2 ml of chloroform was added per 1 ml of TRIzol™ reagent, and the samples were gently inverted and incubated at room temperature for 3 minutes. The samples were then centrifuged at 12,000×g for 15 minutes at 4° C. Following centrifugation, the mixture separates into a lower (red) phenol-chloroform phase, an interphase, and an upper (colorless) aqueous phase, wherein RNA remains exclusively in the aqueous phase. The upper phase was transferred into a fresh tube without disturbing the interphase. For RNA precipitation, 0.5 ml of isopropyl alcohol was added to the tube per 400 μl of the aqueous phase. The samples were incubated at room temperature for 10 minutes and centrifuged at 12,000×g for 10 minutes at 4° C. The supernatant was discarded completely, and the RNA pellet was washed with 1 ml of 75% ethanol, mixed by gently inverting the tube, and centrifuged at 12,000×g for 10 minutes at 4° C. This step was repeated a second time after discarding the ethanol of the first wash, and the samples were then centrifuged at 7,500×g for 5 minutes. The ethanol was removed completely from the samples, and the samples were left to dry in the chemical hood with open tubes for 15-20 minutes.

For bacterial RNA extraction, DH5α E. coli cells transfected with pcDNA3.1+ plasmid were grown overnight in LB medium containing 100 μg/ml ampicillin. The concentration of bacterial cells was determined according to OD at 600 nm, and ˜7·10⁸ cells were taken for RNA extraction. 1 volume of bacterial culture was mixed with 2 volumes of RNAprotect® Bacteria Reagent (QIAGEN), vortexed for 5 seconds and incubated for 5 minutes at room temperature. The sample was then centrifuged for 10 minutes at 5,000×g at room temperature. After discarding supernatant, the pellet was resuspended in 100 μl TE buffer (10 mM Tris-HCl, 1 mM EDTA, pH 8) containing 1 mg/ml lysozyme. The samples were vortexed for 5 seconds, and then incubated at room temperature for 10 minutes with vortexing every 2 minutes. 1 ml TRIzol™ reagent was added to the sample. The sample was incubated at room temperature for 5 minutes, and then RNA extraction was performed as described hereinabove.

### Microscale Thermophoresis:

RNA was extracted from a sample, and after extraction, the RNA pellet was solubilized and hybridized with a specific fluorescently labeled DNA probe in a hybridization solution (as schematically depicted in FIG. 1). After hybridization, the hybridized sample was loaded into an MST capillary and inserted into a Monolith™ MST instrument (NanoTemper Technologies GmbH, Germany). This instrument included a blue mode (493 nm excitation, 650 nm emission) and red mode (521 nm excitation, 670 nm emission) for detection, and an I.R laser (1475 nm+/−15 nm) for heating. The intensities for both fluorescence detection (LED) and IR laser are adjustable, in a range of 0%-100%, wherein the temperature gradient generated has a temperature difference of up to about 2-8° C. Thermophoretic force was induced by infrared laser after 1 second, and maintained for 2 seconds, for a total of a 3 second measurement window (measurement time may optionally be further reduced to 2 seconds using current available MST instruments).

For RNA titration measurements, the extracted RNA solution was diluted to X2 and a serial dilution was prepared in water. Hybridization solutions were prepared as described herein, were X1 solution consisted of SSC buffer X1, 0.05% TWEEN™ 20 surfactant, and 1% formamide unless indicated otherwise. Fluorescently labeled probes were diluted from a 100 μM stock to 1:2400 in hybridization solution X2, denatured for 5 minutes at 100° C. and then incubated on ice for 5 minutes. If a mixture of uniformly labeled probes was prepared, the probes were diluted to a 1:1200 dilution in water and then diluted to 1:2400 with hybridization solution X4 (1:1/number of probes) to create equal molarity of the fluorescent dye. The probe solution was combined with the titration series in a 1:1 (v/v) ratio (10 μL total volume) and allowed to hybridize at a defined temperature for a given time as described. If post-hybridization with intercalating agents (ICA), doxorubicin or Midori™ Green, was performed, the samples were combined with ICA X5 solution 4:1 (v:v) ratio, 8 μL and 2 μL respectively. For cell based measurements, after extraction, the RNA pellet was solubilized and hybridized with a specific fluorescently labeled DNA probe in a hybridization solution (as schematically depicted in FIG. 1).

As depicted schematically in FIG. 2, before applying the thermophoretic force, both free probe and the DNA:RNA hybrid are distributed evenly in space due to mass diffusion; therefore, fluorescence signal is constant. Upon application of a thermophoretic force, both the free probe and the DNA:RNA hybrid migrate along the thermophoretic field away from or into the heating zone. During that period, the flux is controlled by both mass and thermophoretic diffusions; therefore, a change in fluorescence is observed.

As depicted in FIG. 3, fluorescent signals were quantified in the 1 second before application of a temperature gradient (F_{cold}) and between 1 and 2 seconds after application of the temperature gradient (Fₕₒₜ).

### Example 1

### Preparation of Exemplary Microscale Thermophoresis Probes and Proof of Concept

Viral RNA was extracted from a biological sample, and after extraction, the RNA pellet was solubilized and hybridized with a specific fluorescently labeled DNA probe in a hybridization solution, using the procedures described in the Materials and Methods section hereinabove. In initial experiments, the hybridization solution comprised formamide and saline sodium citrate (SSC) buffer solution (X20 SSC; 3 M sodium chloride and 300 mM trisodium citrate, adjusted to pH 7.0 with HCl) and TWEEN™ 20 surfactant (0.05%). After 30 min of hybridization at 42° C. or 62° C., the sample was loaded into a microscale thermophoresis (MST) capillary and placed into an MST instrument.

Formamide is a chemical detergent used in the process of nucleic acid hybridization to lower hybridization temperature and to increase its specificity, which may help to avoid false positive results which could interfere with practical use in clinical diagnosis.

In order to test the feasibility of detecting an extracted viral RNA from a biological sample, a human cell line (U-2 OS) which stably expresses MS2 bacteriophage viral RNA under a doxycycline expression system was used, along with a specific Cy5-DNA probe which emits light in the red spectrum range. In order to determine the lowest amount of probe needed for detection, initial florescence was determined for decreasing concentrations of Cy5-DNA probe in a hybridization solution. A concentration of 100 ng/μL was in the range of the instrument and displayed reliable MST curves (data not shown). In order to model viral infection of human cells, U-2 OS (osteosarcoma) cells were treated with doxycycline for 24 hours in order to initiate MS2 bacteriophage RNA synthesis.

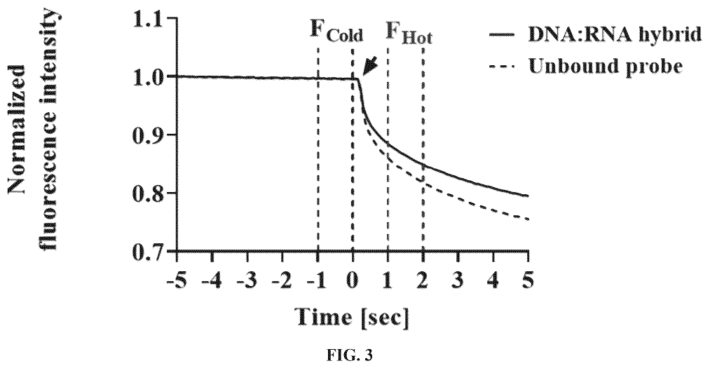

As shown in FIG. 4, increasing concentrations of extracted RNA from treated cells generated a dose-dependent signal, as compared to extracted RNA from untreated cells, which was similar to that of control samples with no RNA.

In order to test biological variance, single point measurements of extracted RNA from different biological samples were performed.

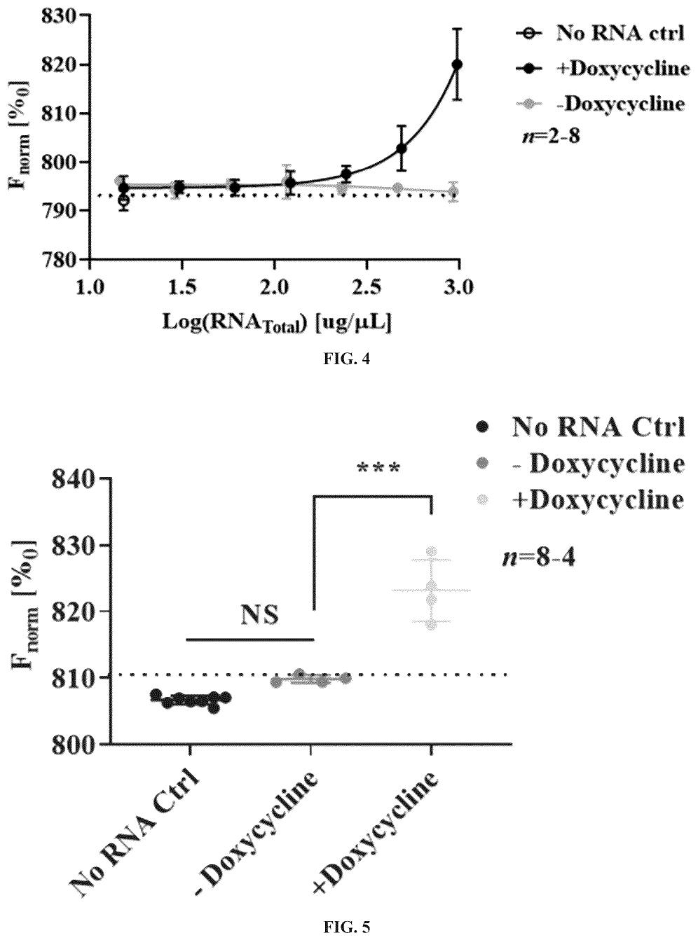

As shown in FIG. 5, the signals of samples containing extracted RNA from treated cells were significantly different from those of untreated extracted RNA and of no RNA control samples.

Taken together, these results indicate that a method described herein can reliably determine the existence of viral RNA in human samples using one-point measurements.

In view of the above, the ability of the method to detect SARS-CoV2 RNA was investigated. As the S-gene of SARS-CoV2 is specific to this virus, a probe was designed to target it. A 1089 bp segment of the S-gene (SEQ ID NO: 1) was cloned into a pcDNA3.1+ plasmid, and a 33 base DNA (SEQ ID NO: 2) tagged with a red florescent dye (S^{Cy5}) was designed according to the complementary RNA sequence to have the highest as possible GC % content for high affinity binding with its target, highest as possible melting temperature, negligible homology to the human genome and no homology to other viruses. Single strand RNA (ssRNA) segments of the S-gene (SEQ ID NO: 1) were then created using an in-vitro transcription system. In addition, a specific probe with SEQ ID NO: 6 was designed which was tagged with a blue fluorescent dye for targeting human 18S rRNA (18S^{ATTO488}) (SEQ ID NO: 5). The structures and properties of the probes are summarized in Table 1 below.

TABLE 1
Structure and physical properties of exemplary DNA probes

| | | | | Length | Tm | GC |
|---|---|---|---|---|---|---|
| Name | SEQ ID NO | Target | Fluorescent dye | (nucleotides) | ° C. | % |
| S^{Cy5} | 2 | S-gene (SARS- | Cy5 (red) | 33 | 79 | 61 |
| | | CoV2) | | | | |
| 18S^{ATTO488} | 6 | 18S rRNA | ATTO 488 (blue) | 46 | 83 | 54 |
| | | (human) | | | | |

As the probes are tagged with different florescent dyes, 18S^{ATTO488} can be used as a control for the RNA extraction process. S^{Cy5} was hybridized with increasing concentrations of in-vitro transcribed S-gene ssRNA, and 18S^{ATTO488} was hybridized with increasing concentrations of human RNA extracted from HEK293T cell line, at different hybridization temperatures. The signals of 18S^{ATTO488} were measured using a blue wavelength acquisition mode and S^{Cy5} using a red wavelength acquisition mode.

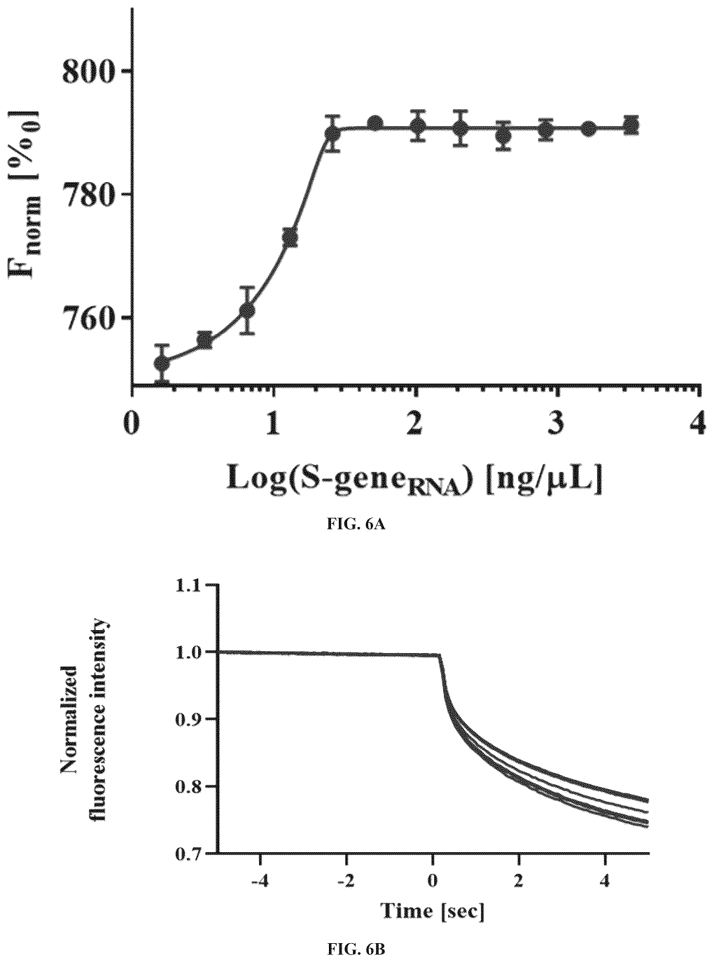

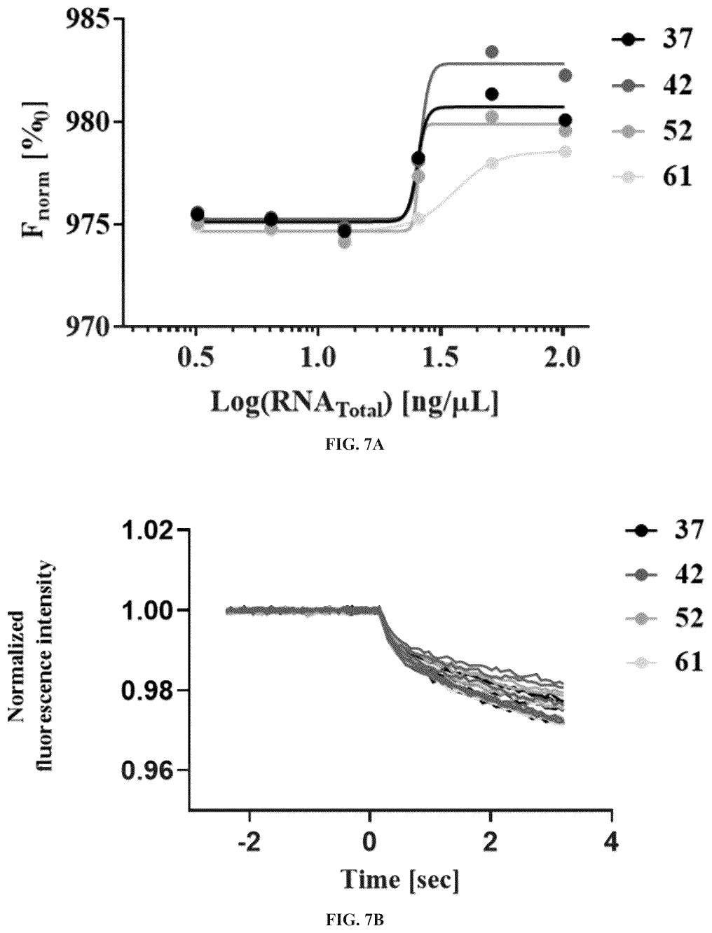

As shown in FIGS. 6A-7B, the fluorescent signal of both the 18S^{ATTO488} probe and the S^{Cy5} probe was dependent on the dose of complementary RNA (18S RNA and S-gene RNA, respectively).

As further shown in FIGS. 7A and 7B, hybridization at 42° C. resulted in particularly large changes in fluorescent signal upon thermophoresis, as compared to lower (37° C.) and higher (52° C. and 61° C.) temperatures.

In order to assess optimal conditions for hybridization, the exemplary S^{Cy5} and 18S^{ATTO488} probes were mixed together and incubated with increasing concentrations of S-gene in the presence of constant concentration of human extracted RNA.

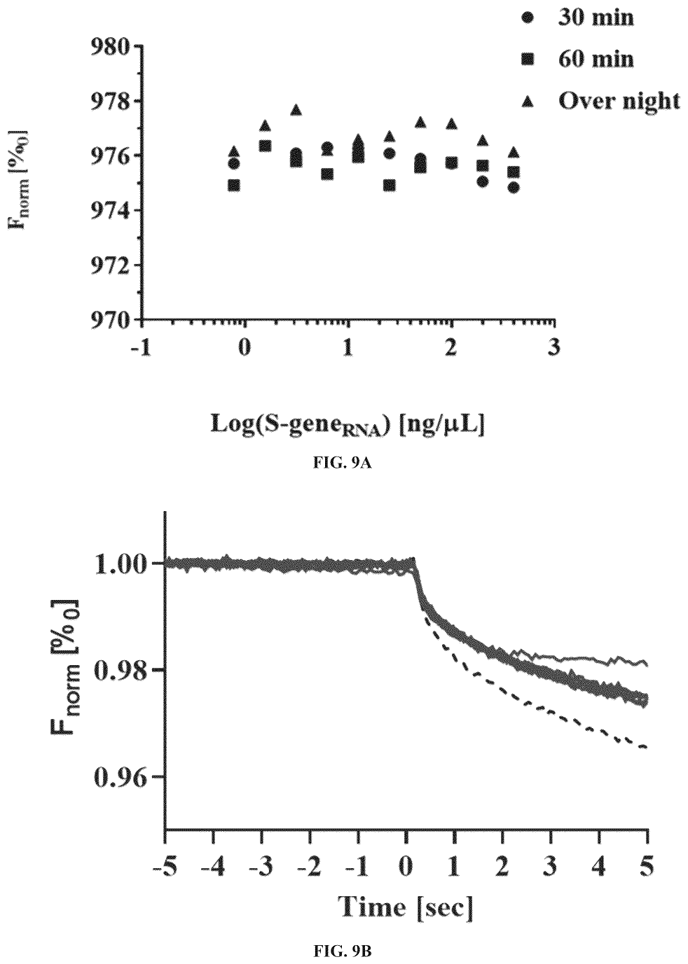

As shown in FIGS. 8A-9B, concomitant detection of S-gene by S^{Cy5} and of 18S rRNA (in human extracted RNA) by 18S^{ATTO488} was effective and quantitative, following hybridization at 42° C. for 30 minutes.

In order to test the selectivity of the S^{Cy5} and 18S^{ATTO488} probes to their targets, the probes were incubated with high concentrations of S-gene (the target of S^{Cy5}), human extracted RNA (comprising the target of 18S^{ATTO488}) or control RNA generated using a pGEM® plasmid to evaluate maximum signal responses.

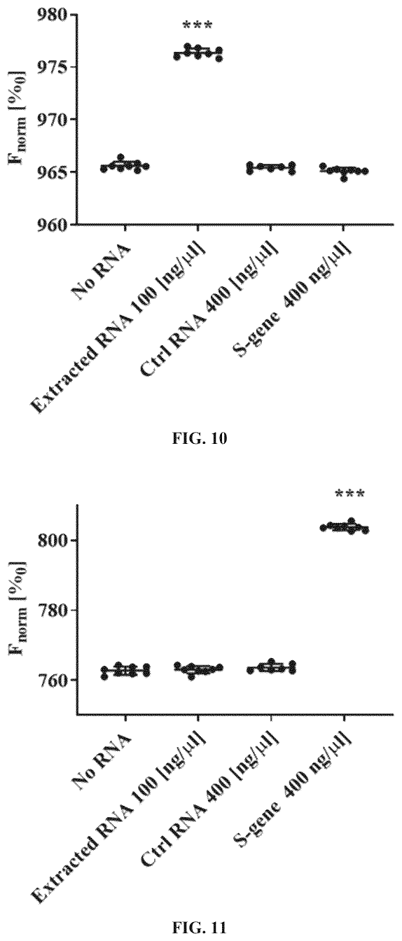

As shown in FIGS. 10 and 11, the S^{Cy5} and 18S^{ATTO488} probes both exhibited selective signal responses to their target RNA.

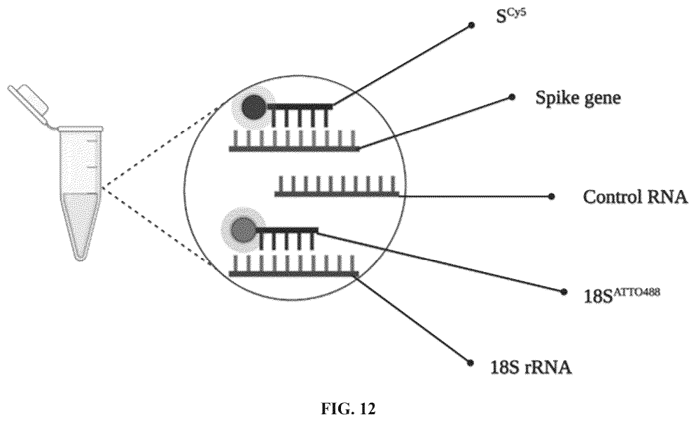

In order to test the ability of an exemplary method to selectively detect the S-gene in a mixture of human RNA, S^{Cy5} and 18S^{ATTO488} probes were incubated together with increasing concentrations of S-gene or control RNA, in the presence of constant concentration of human RNA (as depicted schematically in FIG. 12).

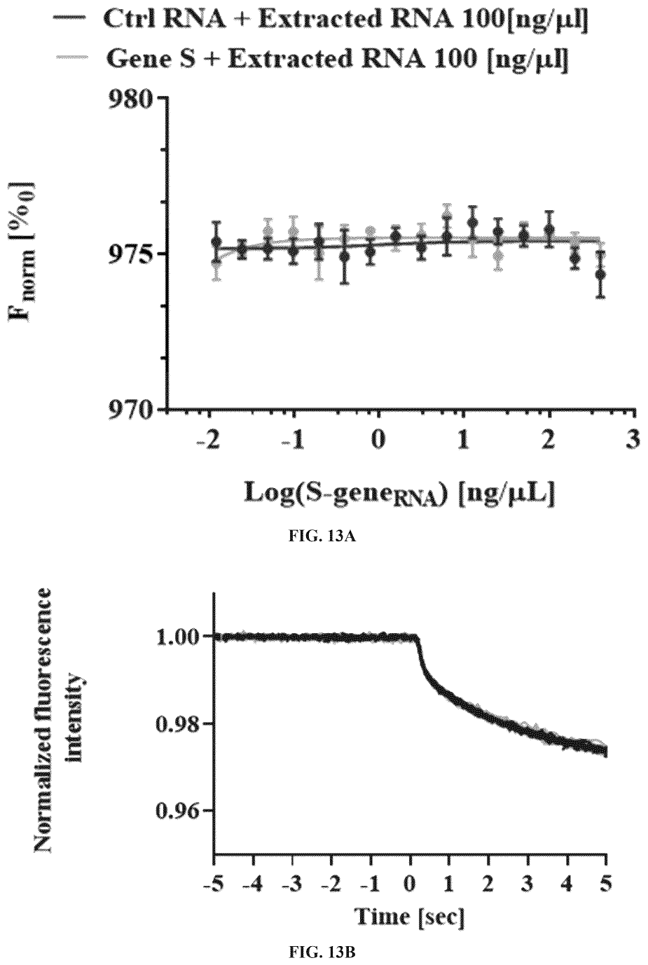

As shown in FIGS. 14A and 14B, increasing concentrations of S-gene RNA, but not control RNA, generated a dose dependent signal, as determined using a red wavelength acquisition mode.

As shown in FIGS. 13A and 13B, all S-gene RNA and control RNA incubations exhibited a constant signal, as determined using a blue wavelength acquisition mode, corresponding to a DNA:RNA hybrid bound state of the 18S rRNA.

These results indicate that pathogens such as SARS-CoV2 can be detected selectively when human RNA is present in solution.

For evaluating the linear dynamic range for this type of measurement, S-gene concentrations were fitted to a linear regression model.

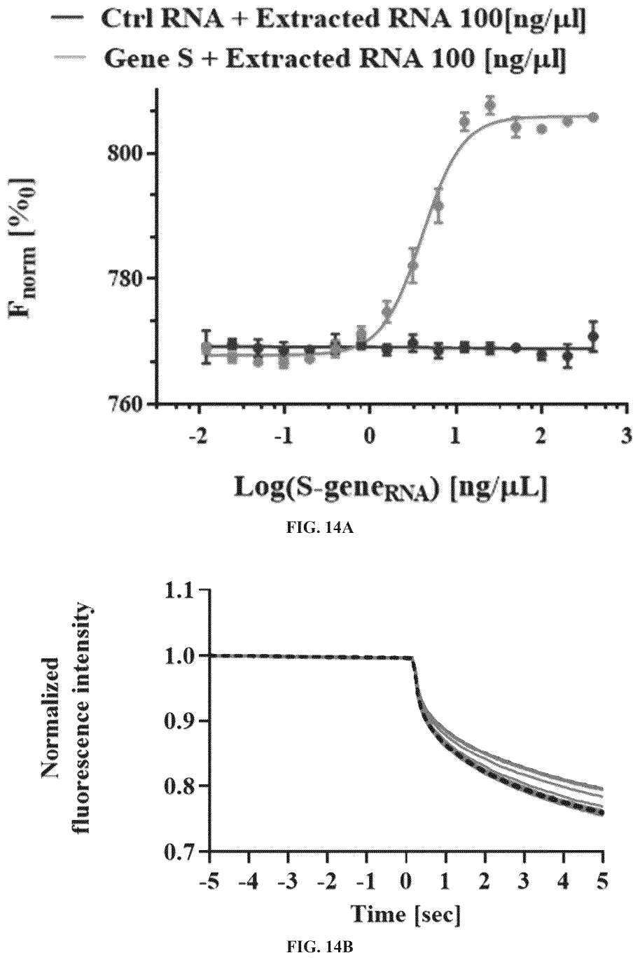

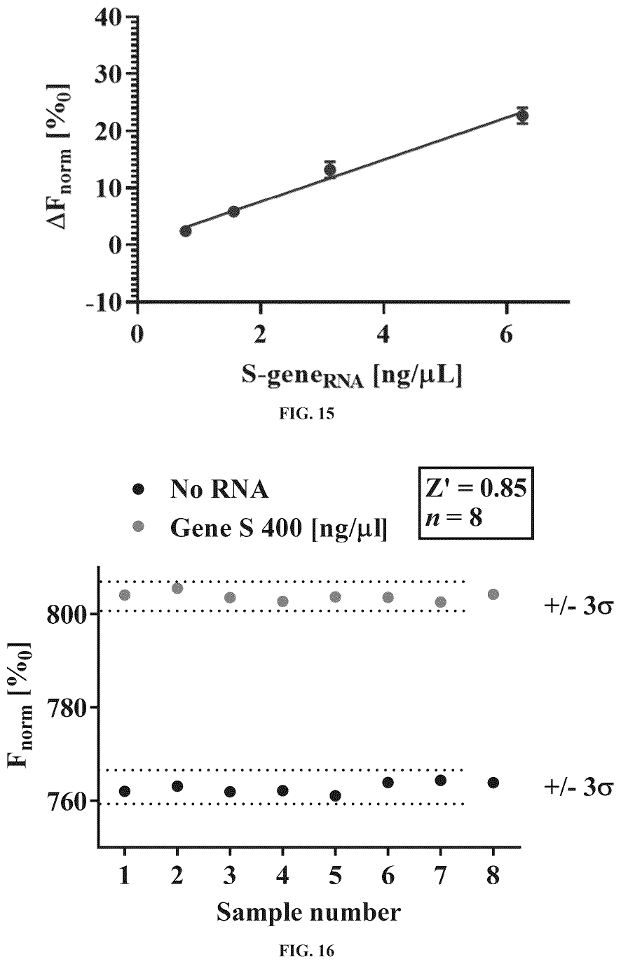

As shown in FIG. 15, a linear correlation was observed in a concentration range between 0.78 to 6.25 ng/μL.

As shown in FIG. 16, the Z′-factor statistical parameter, indicative of assay robustness, was determined to be 0.85 for the maximum signal response of S^{Cy5} probe in the presence of S-gene compared to no RNA control.

These results indicate that the diagnosis method is robust and can be operated in a high-throughput screening approach for screening large portion of the population, using 1-point measurement.

### Example 2

### Effect of Hybridization Conditions on Microscale Thermophoresis Detection of SARS-CoV2 RNA

Hybridization in experiments described in Example 1 were performed in a solution comprising SSC buffer with formamide detergent, where the thermophoretic field caused the migration flux of both S^{Cy5} free probe and the S^{Cy5}:S-gene DNA:RNA hybrid to be directed away from the heating point (as depicted schematically in FIG. 2). The thermophoretic migration of molecules may be affected by size, charge, hydration shell, ionic strength, induced electric fields and heat of transfer capability. In order to further develop a method for clinical diagnosis, the effect of various parameters of the thermophoretic field was investigated.

In one example, the effect of formamide concentration on migration flux was investigated, by incubating S^{Cy5} with increasing concentrations of S-gene in hybridization solution comprising different amounts of formamide (and SSC X2 buffer).

As shown in FIG. 17, the magnitude of the thermophoretic signal response was inversely correlated to formamide concentration.

Without being bound by any particular theory, it is believed that these results may be explained by two possible mechanisms: a) formamide may be competing with DNA:RNA hybridization, thereby weakening the binding interaction; and b) the ability of formamide to create different hydrogen bond networks may affect separation under a temperature gradient, thereby affecting the signal response.

In order to assess the effect of different buffers and buffer concentrations on signal response, samples with SSC buffer, phosphate-buffered saline (PBS) buffer and Tris/NaCl buffer were prepared at various buffer concentrations.

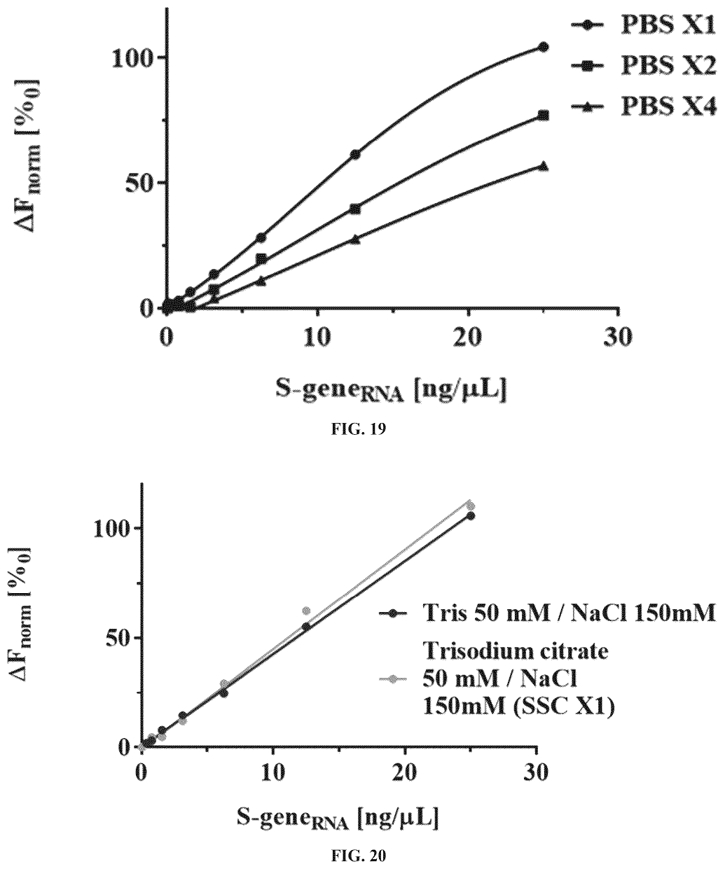

As shown in FIGS. 18 and 19, both SSC and PBS buffers were associated with signal responses which increased upon decreased buffer concentration.

As shown in FIG. 20, Tris/NaCl buffer, which is also used in nucleic acid research, exhibited similar responses to SSC buffer.

These results indicate that salt concentration is a key feature that can affect detection sensitivity. In order to test this assumption, hybridization was performed in pure water.

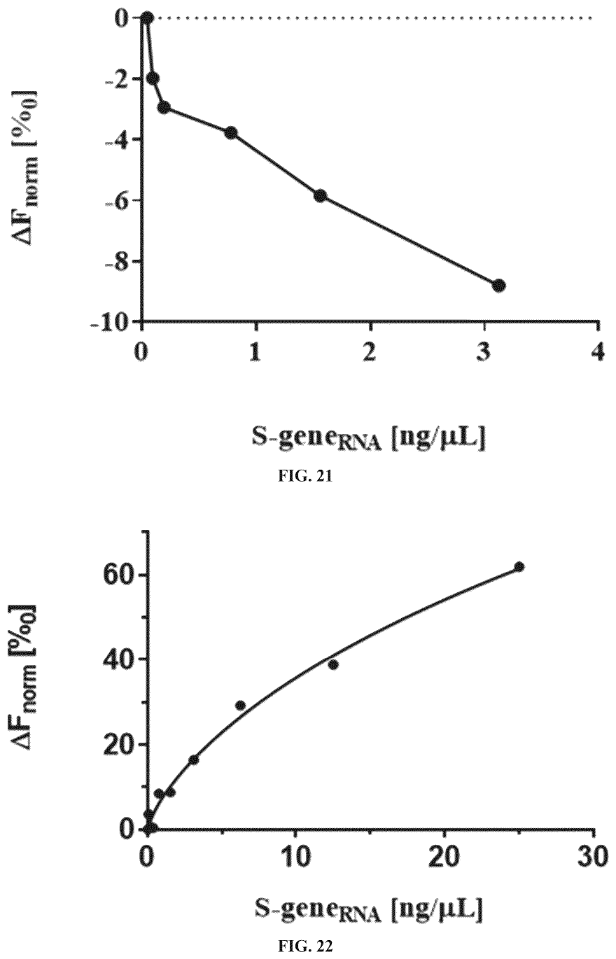

As shown in FIG. 21, a salt-free solution surprisingly reversed the thermophoretic migration flux, causing the DNA:RNA hybrid to migrate further away from the heating zone than the free probe.

In order to determine if NaCl or buffer salts are the cause for the shift in the migration pattern, hybridization was performed in Tris buffer (pH 7.4) with decreasing concentrations of NaCl and Tris alone was used as a control.

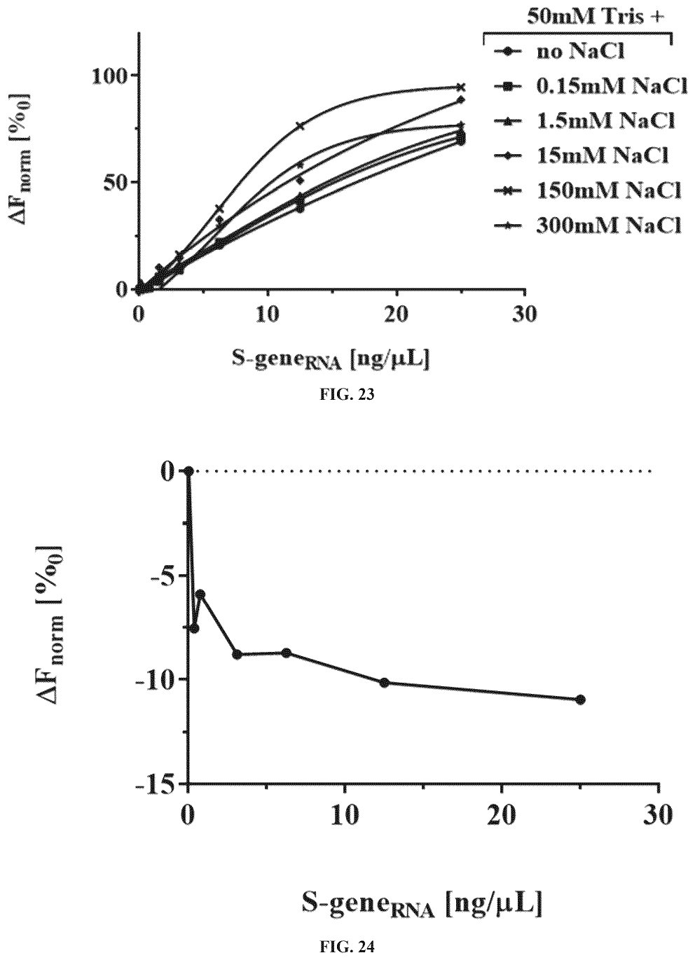

As shown in FIGS. 22 and 23, S^{Cy5} hybridization in Tris solutions with a variety of NaCl concentrations (FIG. 23) resulted in the same thermophoretic behavior as in NaCl solution without Tris (FIG. 22). As further shown in FIG. 23, 150 mM NaCl resulted in the strongest signal response, as compared to NaCl concentrations of 300 mM or of 15 mM or less.

In contrast, as shown in FIG. 24, hybridization in a salt-free solution with a low concentration of Tris buffer resulted in migration flux in the opposite direction.

These results indicate that Tris and NaCl have similar effects on thermophoresis.

As both Tris and NaCl salts contain a chloride atom, hybridization was performed in sodium phosphate buffer without NaCl, in order to assess whether the observed effect is specific or not to chloride ion.

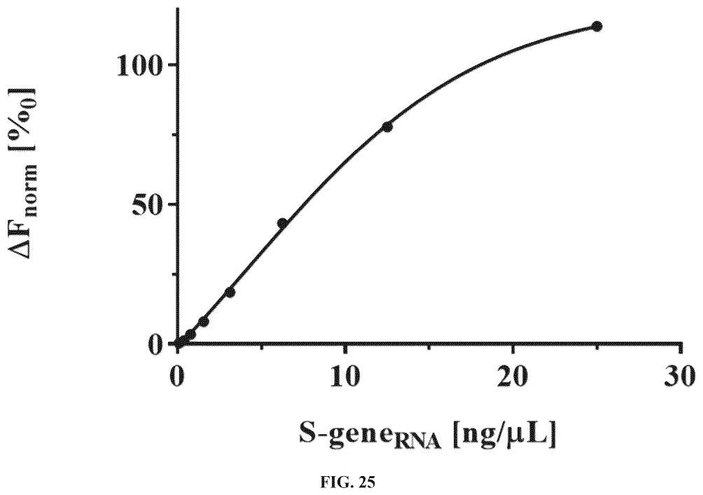

As shown in FIGS. 25, hybridization in sodium phosphate buffer resulted in the same thermophoretic behavior as hybridization in Tris and NaCl solutions.

These results indicate that salts in general control thermophoretic migration pattern. Moreover, as the samples with both pure water and with NaCl solution shared a pH of about 5, and displayed opposite flux patterns, whereas samples with NaCl solution exhibited a migration pattern similar to that of Tris at pH 7, these results indicate that salts, but not pH, are responsible for the reversal of direction of thermophoretic migration.

The thermophoretic behavior upon a given change in temperature (ΔT) is mathematically defined by the Soret coefficient (S_{T}), which is dependent on the entropy of ionic shielding and water hydration shell of a molecule. Under the same force, positive values of the Soret coefficient are associated with molecules drifting out from the heating zone whereas negative values are associated with the molecules entering it, as shown in the following equation:

$$\frac{{F}_{hot}}{{F}_{cold}} = \frac{{C}_{hot}}{{C}_{cold}} = {e}^{- {S}_{T} Δ T} = 1 - {S}_{T} Δ T$$

Without being bound by any particular theory, salts are believed to control the thermophoretic behavior by creating an induced electric field due to the thermophoretic force, altering the ionic strength and electrostatic shielding in solution. It is further believed that both S^{Cy5} free probe and S^{Cy5}:S-gene hybrid drift away from the heating zone, but that their relative migration distances are dependent on the presence of salts, with little regard to the species of salt; such that it is the ionic strength or electrostatic shielding (rather than the induced electric field) which is the cause of the observed effect, possibly due to alteration of the hydration shell of the S^{Cy5} free probe and S^{Cy5}:S-gene hybrid.

In summary, the above results indicate that reduction of the amount of buffer salts and a low concentration of formamide (e.g., from 0% to 10% v/v) are associated with enhances detection sensitivity.

### Example 3

### Simultaneous Testing for SARS-CoV2 and Influenza RNA

As both Covid-19 and influenza are respiratory diseases which share similar symptoms, the ability to diagnose them simultaneously in a single sample was investigated. The HA-gene of H1N1 (annual influenza strain) was selected as a target, as it is unique to the influenza virus, and targeted in current clinical RT-PCR tests.

A 1112 base segment of the HA-gene (SEQ ID NO: 7) and a DNA probe were designed according to procedures such as described above for preparing a probe for SARS-CoV2 S-gene.

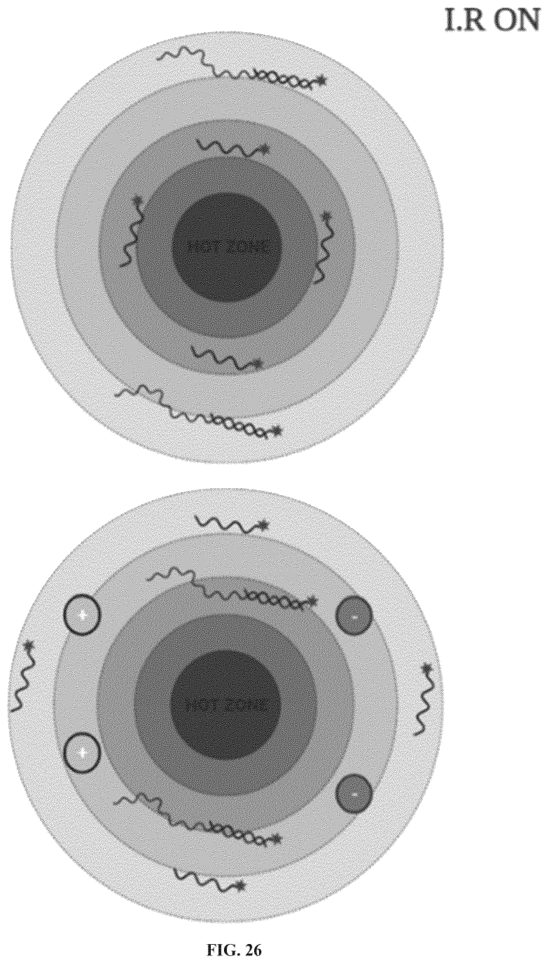

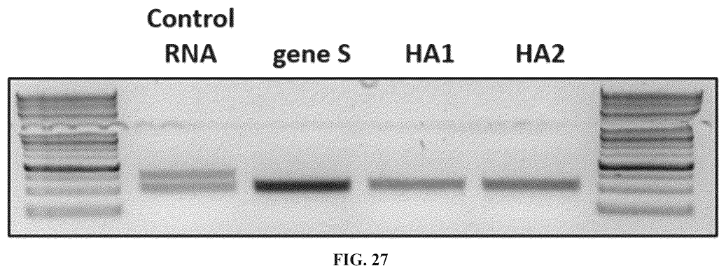

The probe, referred to herein as HA^{ATTO488}, comprised DNA having SEQ ID NO: 8 tagged with a blue fluorescent dye (ATTO 488) to allow the detection of both viruses simultaneously in the same sample using red and blue wavelength acquisition modes. In-vitro transcription of single stranded forms of control RNA, S-gene RNA (SEQ ID NO: 1) and two batches of HA-gene RNA (SEQ ID NO: 7) was confirmed by gel electrophoresis (as shown in FIG. 27).

The HA^{ATTO488} probe was first hybridized with increasing concentrations of HA-gene RNA in a hybridization solution comprising different concentrations of formamide and SSC buffer, in order to assess its optimal hybridization conditions.

As shown in FIGS. 28A and 28B, increasing concentrations of HA-gene generated a dose-dependent signal, wherein the fluxes of the free probe and of the HA^{ATTO488}:HA-gene (DNA:RNA) hybrid were surprisingly directed towards the heating zone with decreasing concentrations of formamide.

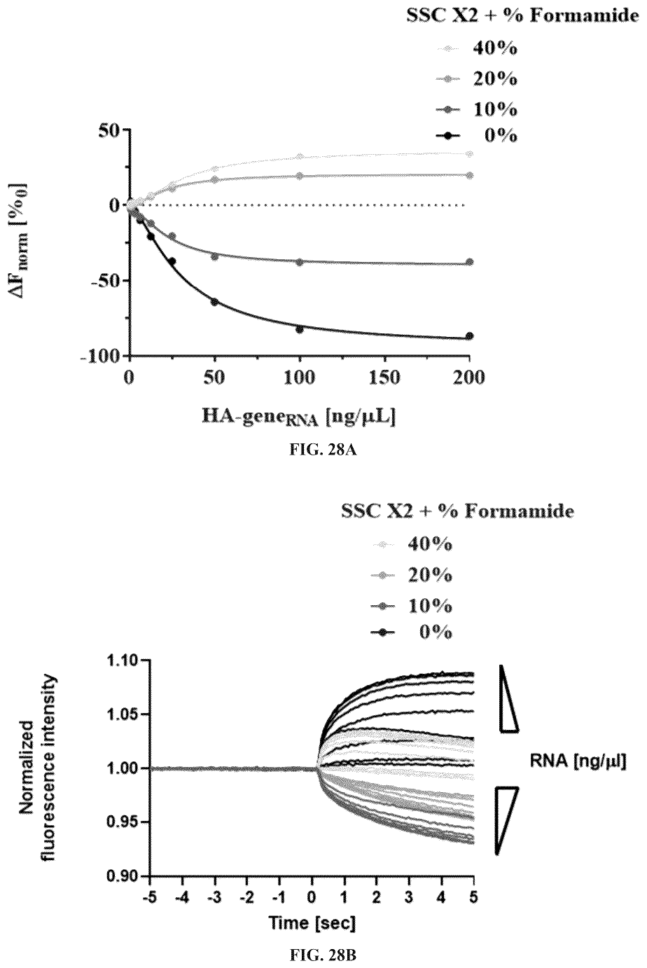

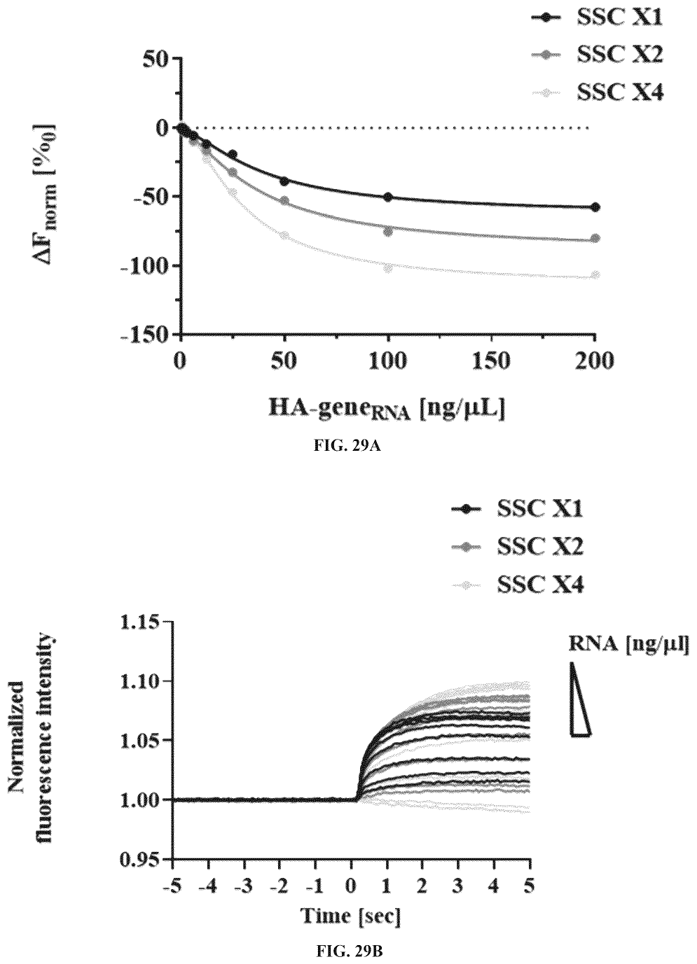

Moreover, as shown in FIGS. 29A and 29B, higher concentrations of saline sodium citrate buffer increased detection sensitivity for HA-gene using HA^{ATTO488} probe in a dose-dependent manner; as opposed to results for the Cy5-tagged probe (FIGS. 17 and 18).

The ability to use both probes simultaneously (co-incubated) was investigated. To this end, experiments were performed in a solution of 10% (v/v) formamide and SSC X1, in order to facilitate better detection for both probes, yet avoiding unspecific hybridizations. In order to test specificity under these conditions, HA^{ATTO488} was incubated with increasing concentrations of HA-gene, S-gene or control RNA.

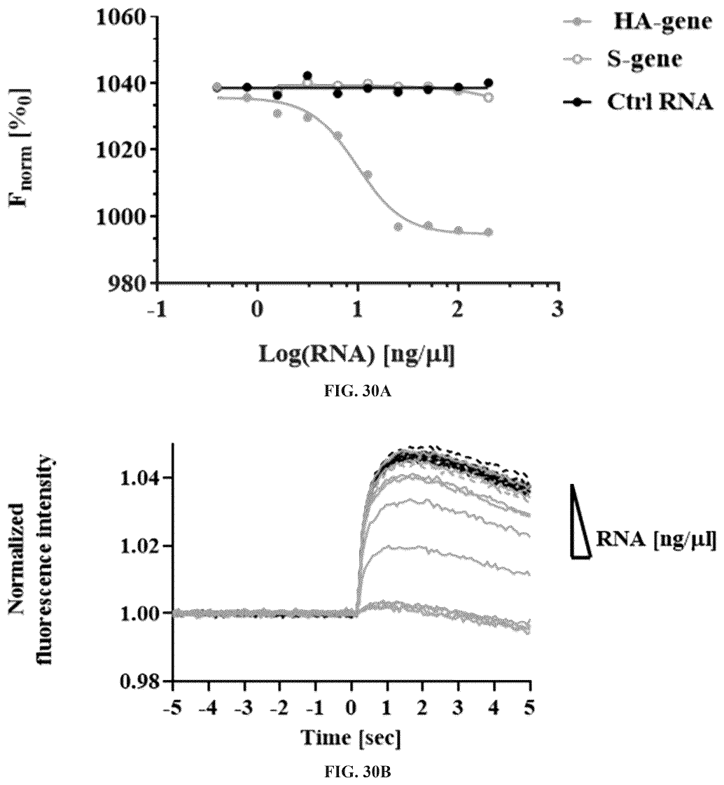

As shown in FIGS. 30A and 30B, the signal of the HA^{ATTO488} probe was dependent on the concentration of HA-gene but not on the concentration of S-gene or control RNA.

Reciprocally, when S^{Cy5} probe was incubated with increasing concentrations of HA-gene no response in signal was observed when measured using the red wavelength acquisition mode (data not shown).

In order to test the reliability of exemplary probes for detecting both viruses stimulatingly, S^{Cy5} and HA^{ATTO488} probes were incubated together with a mix of increasing concentrations of HA-gene and S-gene RNA segments or control RNA, in hybridization solution comprising 10% (v/v) formamide and saline sodium citrate (X1) buffer. The structures and properties of the two probes are summarized in Table 2 below.

TABLE 2
Structure and physical properties of exemplary DNA probes

| | DNA | | Fluorescent | Length | | GC |
|---|---|---|---|---|---|---|
| Name | sequence | Target | dye | (nucleotides) | Tm | % |
| S^{Cy5} | SEQ ID NO: 2 | S-gene | Cy5 | 33 | 79° C. | 61% |
| | | (SARS-CoV2) | (red) | | | |
| HA^{ATTO488} | SEQ ID NO: 8 | HA gene | ATTO488 | 33 | 81° C. | 61% |
| | | (H1N1 influenza) | (blue) | | | |

As shown in FIGS. 31A and 31B, increasing concentrations of each of the genes generated a dose-dependent signal, specific to the respective probe (and wavelength acquisition mode), 10% (v/v) formamide and saline sodium citrate (X1) buffer; whereas no change in signal was observed with increasing concentrations of control RNA with either blue or red wavelength acquisition mode.

In order to further improve sensitivity, the measurements were repeated in a solution of 1% (v/v) formamide and saline sodium citrate (X1) buffer.

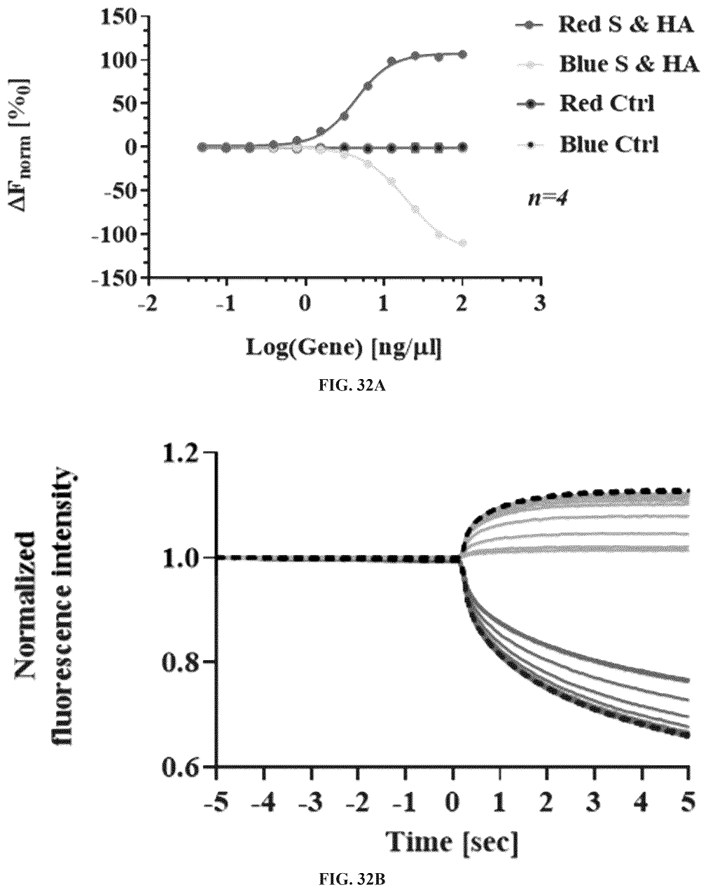

As shown in FIGS. 32A and 32B, increasing concentrations of each of the genes generated a dose-dependent signal, specific to the respective probe (and wavelength acquisition mode), in 1% (v/v) formamide and saline sodium citrate (X1) buffer; whereas no change in signal was observed with increasing concentrations of control RNA with either blue or red wavelength acquisition mode.

For evaluating the linear dynamic range, the S-gene and HA-gene signal responses were fitted to linear regression models.

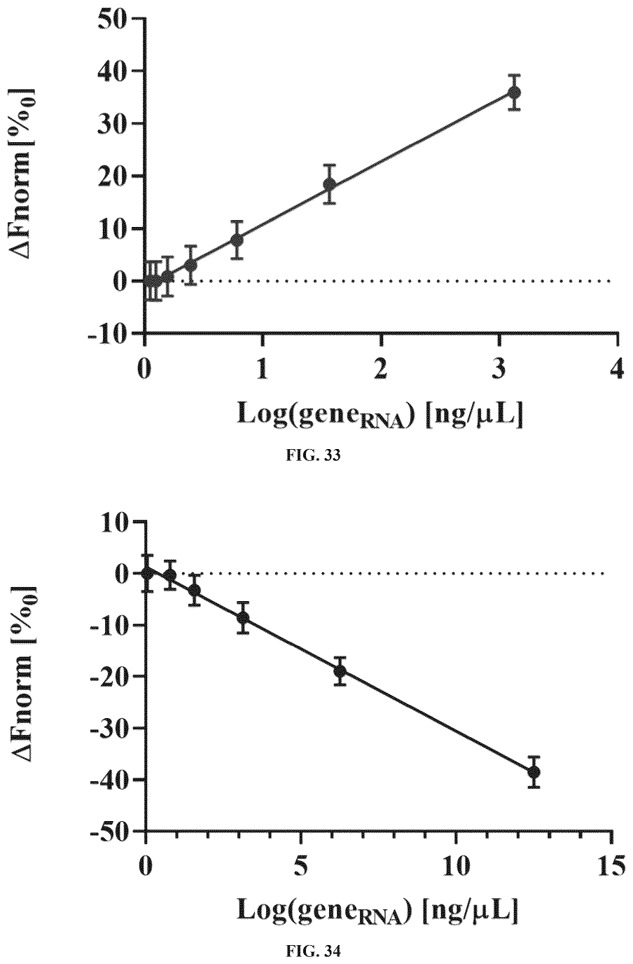

As shown in FIGS. 33 and 34, linear correlations were observed in a concentration range between 0.098 to 3.125 ng/μL for S-gene (FIG. 33) and between 0.78 to 12.5 ng/μL for HA-gene (FIG. 34).

During the abovementioned measurements it was observed that hybridization at 62° C. for 10 minutes was sufficient for reaching steady-state binding, and therefore these conditions were used for the following experiment.

Taken together, the above results indicate that embodiments of the invention can be used for specific detection of two different RNA targets simultaneously, while minimizing false positive results.

The results herein indicate that S^{Cy5} and HA^{ATTO488} probes exhibit different thermophoretic behaviors.

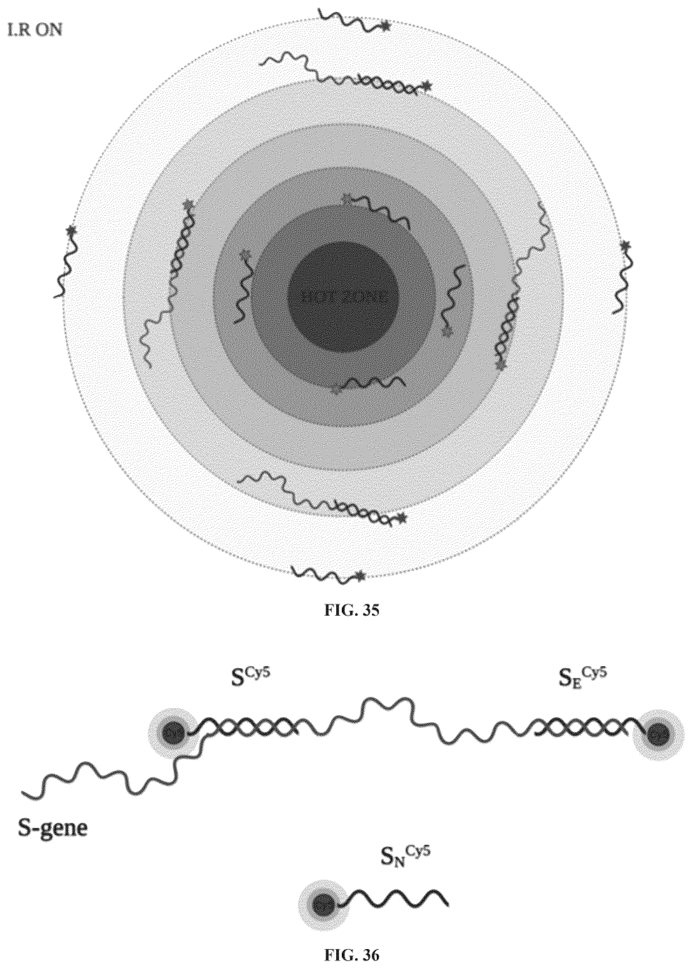

FIG. 35 schematically depicts the distribution of both free probes and DNA:RNA hybrids under a thermophoretic force, as observed under optimized conditions.

Without being bound by any particular theory, it is believed that as both RNA targets (S-gene and HA-gene) are about 1 kb in length, and both matching probes are of the same length and share similar physicochemical properties, the observed differences in thermophoretic migration are a result of the differences in structure of the attached fluorescent dye, Cy5 or ATTO488. It is further believed that small molecules could be used for controlling the thermophoretic migration fluxes of designed probes, which may affect the separation of the free probe from the DNA:RNA hybrid, thereby improving detection sensitivity.

### Example 4

### Effect of Probe Binding Location and Combinations of Probes on RNA Detection Sensitivity

Hybridization according to procedures described hereinabove occurred at the middle of the in-vitro transcribed target gene. The location in the target RNA which binds to the probe may affect thermophoretic migration. In addition, the signal recorded is the average flux of the labeled molecules; therefore if more than one probe binds to the target gene at the same time while the probes are tagged with the same fluorescent dye, it may enhance detection at lower RNA levels. In order to investigate the effect of simultaneously using multiple probes, which bind to different locations at the same target gene, two reverse complement sequence DNA probes tagged with Cy5 were tested: the probe described hereinabove, referred to as S^{Cy5}, which binds at the middle of the target gene; and a probe designed to bind at the edge of the target gene, referred to herein as S_{E}^{Cy5}, comprising DNA having SEQ ID NO: 3. In addition, a “non-binding” probe (S_{N}^{Cy5}) comprising DNA having SEQ ID NO: 4 (which exhibits no change in MST signal, despite having a complementary sequence to the S-gene under the tested conditions, indicating that it does not bind or binds but does not affect migration) serves as a negative control (as depicted in FIG. 36) was tested. The structures and properties of the probes are summarized in Table 3 below. The aforementioned probes were incubated together or separately with S-gene RNA.

TABLE 3
Structure and physical properties of exemplary
DNA probes complementary to SARS-CoV2 S-gene

| | DNA | Fluorescent | Length | | |
|---|---|---|---|---|---|
| Name | sequence | dye | (nucleotides) | Tm | GC % |
| S^{Cy5} | SEQ ID | Cy5 (red) | 33 | 79° C. | 61% |
| | NO: 2 | | | | |
| S_{E}^{Cy5} | SEQ ID | Cy5 (red) | 22 | 64° C. | 45% |
| | NO: 3 | | | | |
| S_{N}^{Cy5} | SEQ ID | Cy5 (red) | 22 | 66° C. | 50% |
| | NO: 4 | | | | |

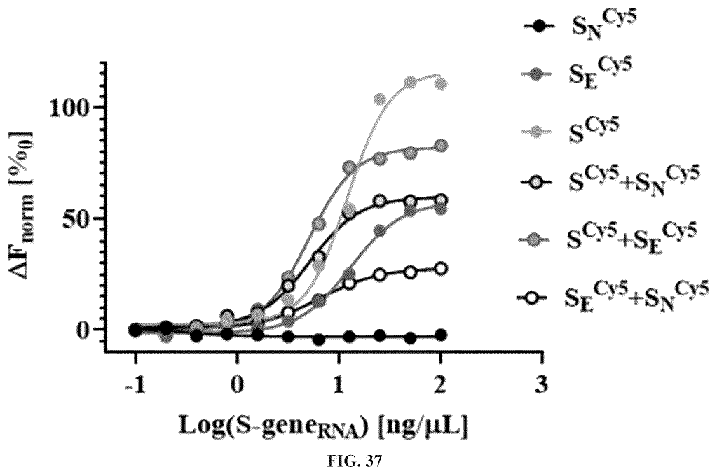

As shown in FIG. 37, both S^{Cy5} and S_{E}^{Cy5} exhibited a thermophoretic signal dependent on S-gene RNA concentration), whereas no dependence of the S_{N}^{Cy5} signal on S-gene concentration was observed.

These results confirm that both S^{Cy5} and S_{E}^{Cy5} bind the S-gene, whereas the S_{N}^{Cy5} negative control does not.

As further shown in FIG. 37, S^{Cy5} exhibited a stronger response than did S_{E}^{Cy5} (ΔFₙₒᵣₘ=110.63‰ versus 54.41‰); and that when incubated together, S^{Cy5} and S_{E}^{Cy5} exhibited a better response than did the other combinations excluding S^{Cy5}. As further shown therein, S_{N}^{Cy5} did not generated dose-dependent signals and decreased the observed response when incubated with either S^{Cy5} or S_{E}^{Cy5}.

These results, including the difference in maximum responses between S^{Cy5} and S_{E}^{Cy5}, suggest that after hybrid formation, the change in thermophoretic parameters is dependent on the binding location on the target probe.

In order to evaluate responses at lower RNA levels, linear regression models were created and the slopes were compared. The slopes indicate the ability to distinguish between different analyte concentrations, and can be used to evaluate analytical sensitivity.

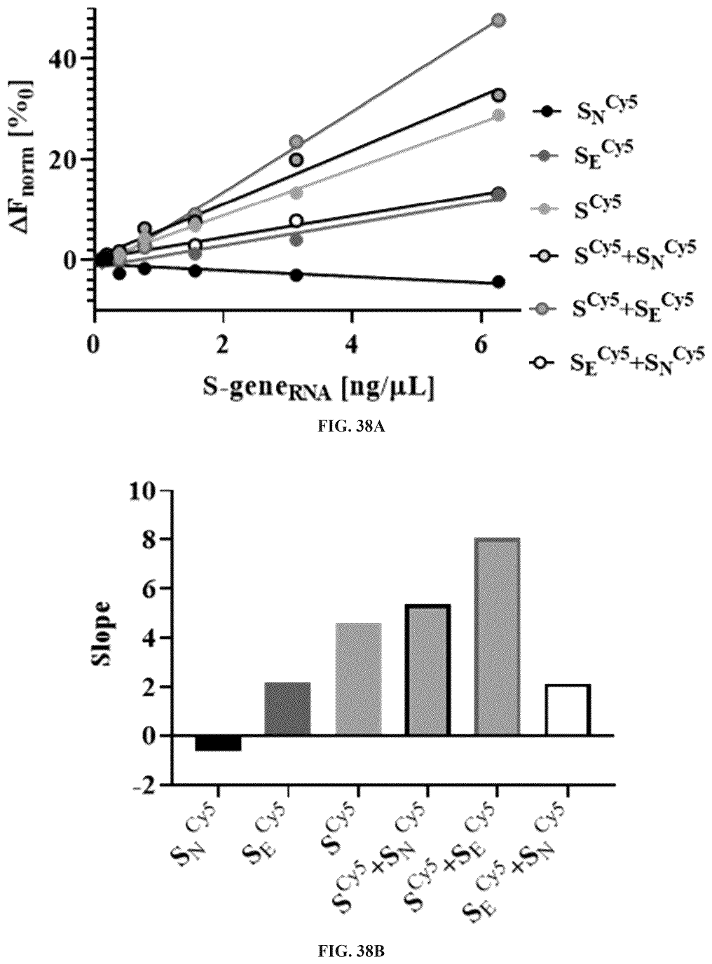

As shown in FIGS. 38A and 38B, the mix of S^{Cy5} and S_{E}^{Cy5} exhibited the steepest slope, indicating that a mix of probes which target multiple regions on the target RNA can significantly enhance detection sensitivity.

### Example 5

### Effect of Intercalating Agents on RNA Detection Sensitivity

As discussed in Example 3, it was found during simultaneous measurements of SARS-CoV2 and Influenza H1N1 that differences in the thermophoretic migration of the two probes are associated with different fluorescent dyes. This suggest that migration patterns could be further controlled using different fluorophores or additional binders. It was hypothesized that intercalating agents (ICA) which bind ssRNA/DNA and dsRNA/DNA nonspecifically could be used as detection enhancers when incubated with the nucleic acid after hybridization. In particular, as intercalating agents bind nucleic acids with higher affinity towards dsDNA/RNA, introducing them after the hybridization step may induce a more evident thermophoretic difference between the free probe and the DNA:RNA hybrid, thereby improving sensitivity of detection. In order to investigate this possibility, the effects of two intercalating agents were tested and compared: 1) Midori™ Green (MG), a mixture of molecules which bind double-stranded and single-stranded nucleic acids and which are used to stain and visualize DNA, and 2) doxorubicin (Dox), a common chemotherapy agent which binds to double strand nucleic acids with higher affinity than to single strand nucleic acids, and is used to treat cancer. As MG and Dox share blue excitation and emission spectrums, both acquisition wavelengths, red and blue, could be used to study the crosstalk between S^{Cy5} probe and the intercalating agents.

Initially, S^{Cy5} was hybridized with increasing concentrations of S-gene RNA, and afterwards the samples were divided and incubated with increasing amounts of MG. As MG stock concentration is not reported, the concentration of MG was characterized by the degree of dilution of the stock concentration.

As shown in FIGS. 39A and 39B, the MG thermophoretic migration flux (as determined using the blue wavelength acquisition mode) was directed away from the heating zone; whereas for a 1:1000 MG dilution, a complete (sigmoid) binding curve was observed, with increasing concentrations of S-gene generating a dose-dependent signal.

In addition, initial fluorescence intensity values were increased at relatively high concentrations of MG, but most of the data points were within the range of MST quantification.

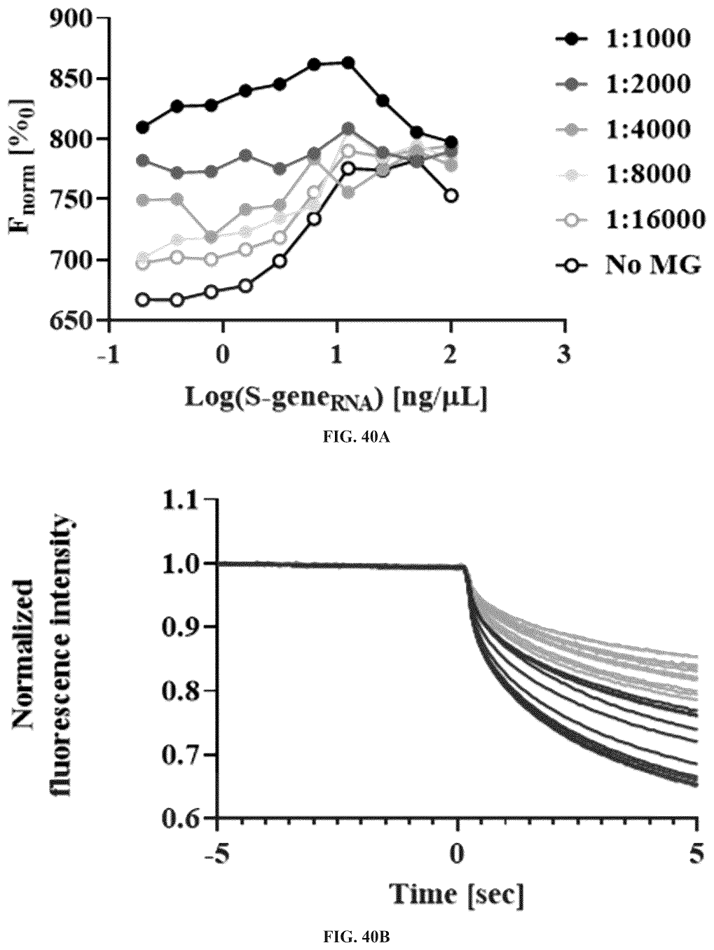

Similarly, as shown in FIGS. 40A and 40B, S^{Cy5} thermophoretic migration (as determined using the red wavelength acquisition mode) was directed away from the heating zone, and most of initial fluorescence values were within the range of MST quantification for all MG concentrations (data not shown).

As shown in FIG. 40A, MG was associated with a decrease in MST signal response up to a dilution of 1:2000. These results suggest that MG alters the thermophoretic migration which affected the average flux.

However, as further shown therein, at an MG dilution of 1:1000 (which exhibited a complete binding curve), an increase in signal response (Fₙₒᵣₘ) was observed at relatively low RNA concentrations, whereas at higher RNA concentrations, the signal plateaus and then decreases to a signal level which is similar to the control with no MG.

Taken together, these results suggest that MG is altering the thermophoretic migration flux of both the free probe and the hybrid after binding, directing the hybrid closer to the heating zone. Furthermore, as MG binds nonspecifically to both double-stranded and single stranded nucleic acids, the increase in RNA concentrations competes with S^{Cy5} and S^{Cy5}:S-gene hybrid over MG binding, allowing the flux to become similar to the control group with no MG.

In order to evaluate the analytical sensitivity, the slopes of the linear regression models were compared.

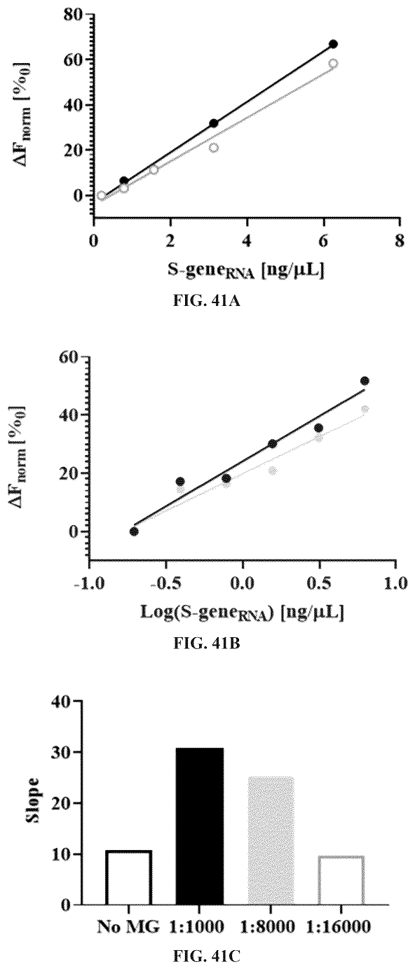

As shown in FIG. 41A, samples with no MG and with highly diluted MG (1:16000) exhibited similar linear fits to RNA concentration, using a linear scale.

Similarly, as shown in FIG. 41B, samples with MG at dilutions of 1:1000 and 1:8000 exhibited similar linear fits to RNA concentration, using a logarithmic scale.

In contrast, samples with MG dilutions of 1:2000 and 1:4000 could not be fitted linearly on either a linear scale or a logarithmic scale.

As shown in FIG. 41C, samples with MG at dilutions of 1:1000 and 1:8000 exhibited considerably steeper slopes than did samples with more diluted (1:16000) MG or no MG, indicating that MG enhances analytical sensitivity.

In order to investigate the effect of Dox on detection sensitivity, S^{Cy5} was hybridized with increasing concentrations of S-gene RNA, then the samples volumes were evenly divided to two sets, where one set was incubated with increasing concentrations of Dox and the other set was served as control.

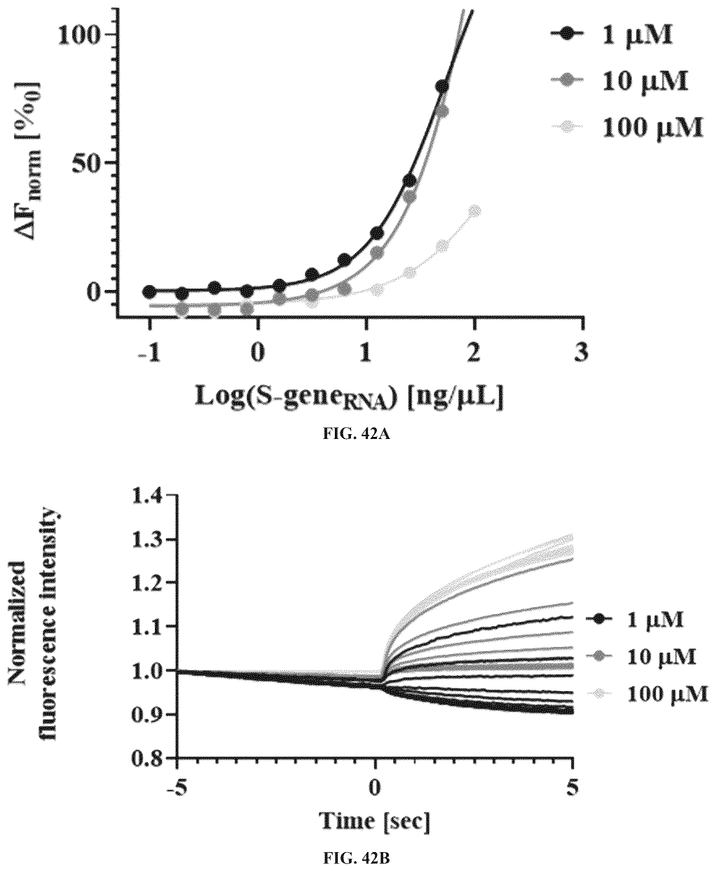

As shown in FIGS. 42A and 42B, Dox fluorescence (at blue wavelengths) at all tested concentrations was dependent on S-gene RNA concentration, with different Dox concentrations exhibiting different degrees of dependence on S-gene RNA concentration.

As shown in FIG. 42B, Dox thermophoretic migration (as determined by changes in blue fluorescence) was directed away from the heating zone, whereas increasing concentrations of S-gene generated a dose-dependent signal at all Dox concentrations to various degrees (as indicated by the proximity of the MST curves). 1 μM and 10 μM Dox MST curves were similar to each other, yet indicated Dox migration away from the heating zone, except at increased concentrations of S-gene; whereas surprisingly, 100 μM Dox MST curves all exhibited an increase in fluorescence, indicating thermophoretic migration towards the heating zone regardless of S-gene concentration. In addition, the MST curves of 100 μM Dox were in closer proximity to one another than the curves for 1 μM and 10 μM, e.g., as represented by ΔFnorm values.

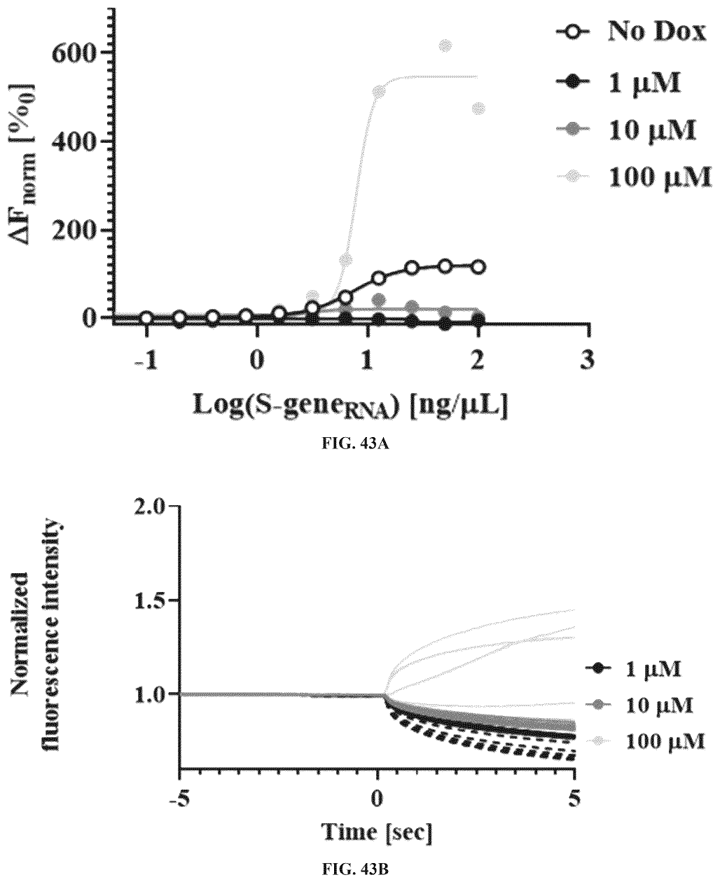

As shown in FIG. 43B, S^{Cy5} fluorescence (at red wavelengths) decreased upon application of a heating gradient in the absence of Dox, and Dox reversed this phenomenon in a dose-dependent manner, with 1 or 10 μM Dox resulting in a smaller decrease of S^{Cy5} fluorescence, and 100 μM Dox resulting in an increase of fluorescence.

As shown in FIGS. 43A and 43B, at all Dox concentrations, S^{Cy5} fluorescence was dependent on S-gene RNA concentration, although with considerably different signal responses. As shown in FIG. 43A, the greatest signal response was for 100 μM Dox, with average maximum ΔFₙₒᵣₘ values of 547.1‰, as compared to 116.5‰ for samples with no Dox.

These results indicate that Dox induces S^{Cy5}:S-gene to flow towards the heating zone in a dose-dependent manner.

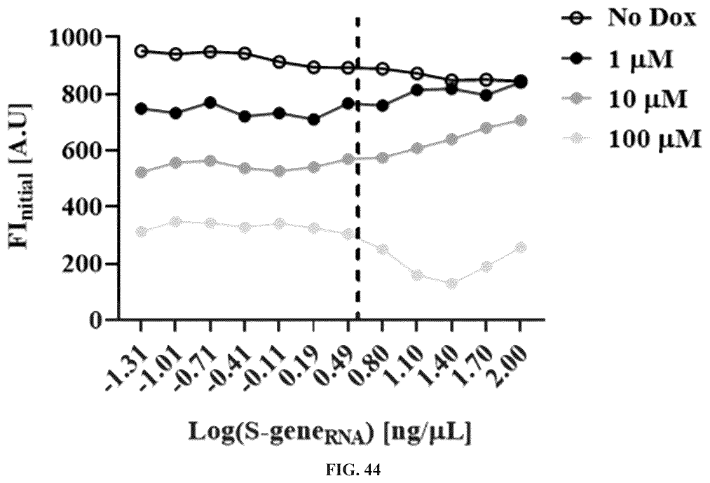

As shown in FIG. 44, Dox decreased the initial fluorescence intensity values (prior to applying a temperature gradient) of S^{Cy5} in a dose-dependent manner; and at relatively high RNA concentrations, the decrease in initial fluorescent intensity was particularly greater in the presence of 100 μM Dox.

As further shown in FIG. 44, the initial fluorescence intensity values of the first 7 data points (for the 7 lowest RNA concentrations) were within 10% variability.

This indicates that such data points may be useful for evaluating MST curves, while avoiding artifacts associated with binding-induced quenching. The signal responses for the 7 lowest RNA concentrations were therefore fitted to a linear regression model and the slopes were compared.

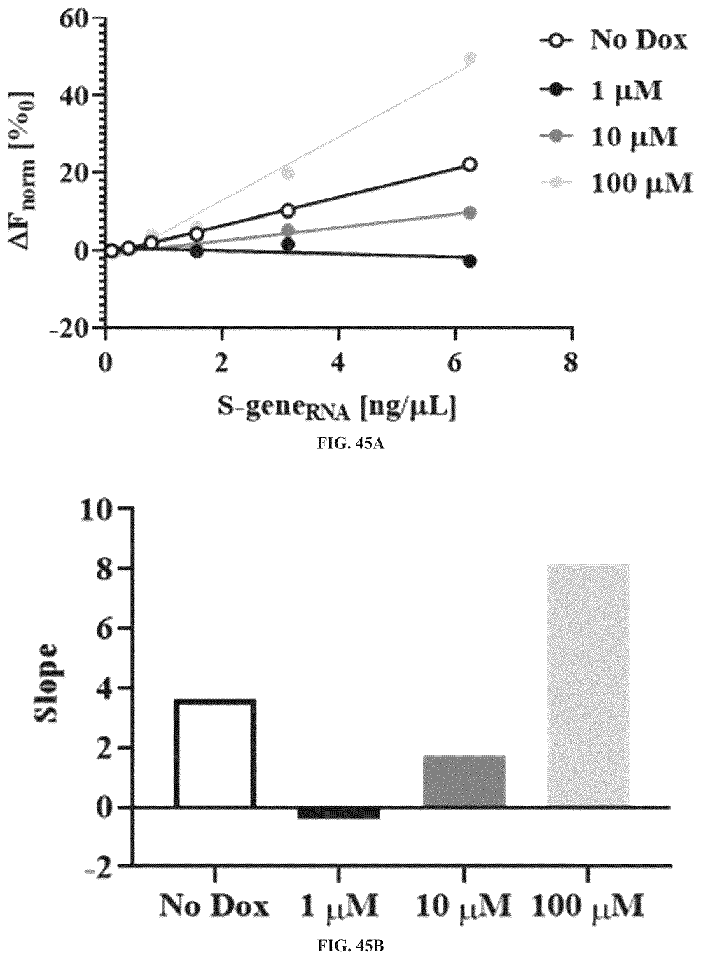

As shown in FIGS. 45A and 45B, samples with 100 μM Dox exhibited a steeper slope (greater dependence of the signal on RNA concentration) than did samples with 0, 1 or 10 μM Dox, indicating that Dox at concentrations such as 100 μM enhanced analytical sensitivity.

In order to investigate if the effect of Dox depends on DNA:RNA formation, 100 μM Dox was incubated with various concentrations of S-gene RNA in hybridization solution, with no S^{Cy5} probe.

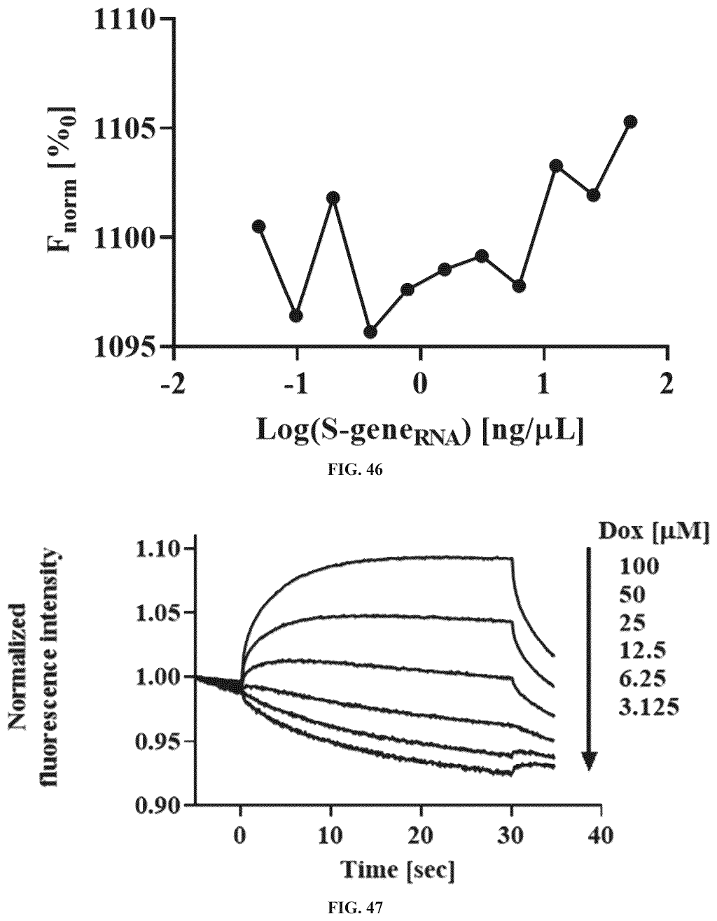

As shown in FIG. 46, S-gene RNA concentration did not noticeably affect the change in signal in the absence of the DNA probe. This result indicates that the effect of Dox is dependent on DNA:RNA formation.

In order to investigate the possibility that Dox monomers interact with each other, thereby causing Dox to migrate into the heating zone particularly at high Dox concentrations, Dox was incubated in hybridization solution without the presence of either S-gene RNA or the DNA probes.

As shown in FIG. 47, the degree of Dox migration into the heating zone was correlated to Dox concentration.

This result indicates that self-interaction occurs between Dox monomers in a dose-depended manner, regardless of the presence or absence of RNA or the DNA probes.

In order to determine whether the effect of Dox is specific towards S^{Cy5}:S-gene hybrid formation, S^{Cy5} was incubated with increasing concentrations of S-gene or control RNA, then the samples volumes were evenly divided to two sets, where one set was incubated with 100 μM Dox and the other set was served as control.

Both S-gene RNA and control RNA generated similar dose-dependent signals of Dox fluorescence (data not shown).

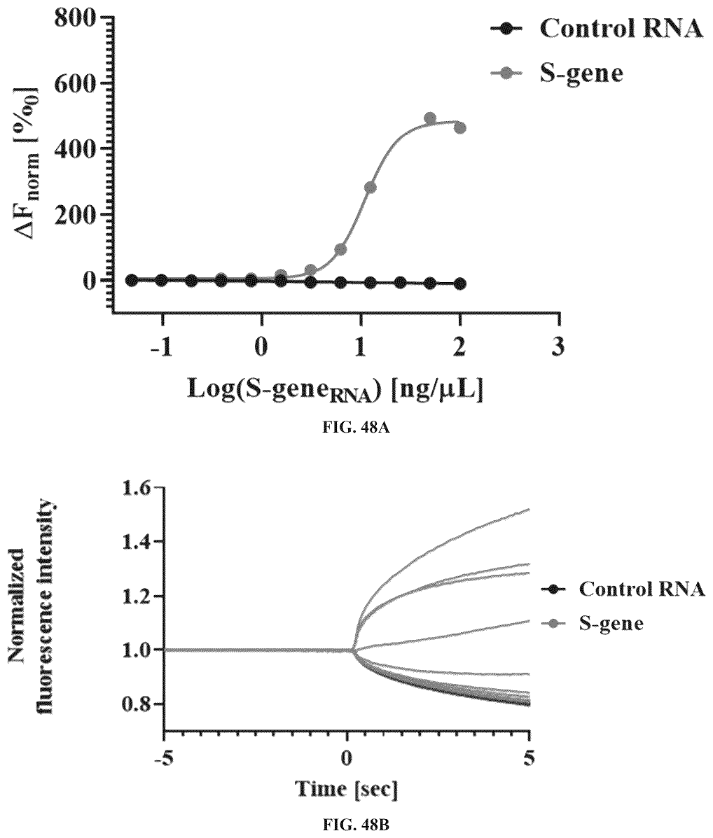

As shown in FIGS. 48A and 48B, in the presence of 100 μM Dox, the thermophoretic signal of S^{Cy5} (as determined using the red wavelength acquisition mode) was altered by S-gene RNA in a dose-dependent manner; whereas control RNA had no effect on the S^{Cy5} signal. Furthermore, only the thermophoretic flux of S^{Cy5} in the presence of relatively high S-gene RNA concentrations was directed towards the heating zone; whereas S^{Cy5} at relatively low RNA concentrations behaved similarly to S^{Cy5} in the presence of control RNA, with the thermophoretic flux being away from the heating zone.

Taken together, the above results indicate that addition of binding (e.g., intercalating) agents can enhance detection sensitivity.

### Example 6

### Combined Effect of Intercalating Agents and Multiple Probes on RNA Detection Sensitivity

As discussed in Example 5, Dox (doxorubicin) and MG (Midori™ Green) each exhibited an ability to enhance analytical sensitivity. However, Dox also exhibited selective responses towards DNA:RNA hybrids, and was therefore selected for further experiments, in which the combined effect of Dox and the use of multiple probes (e.g., as discussed in Example 4) was investigated. To this end, various Cy5-labeled probes (described in Example 4) were hybridized with S-gene RNA (at various RNA concentrations) and thereafter incubated with 100 μM Dox.

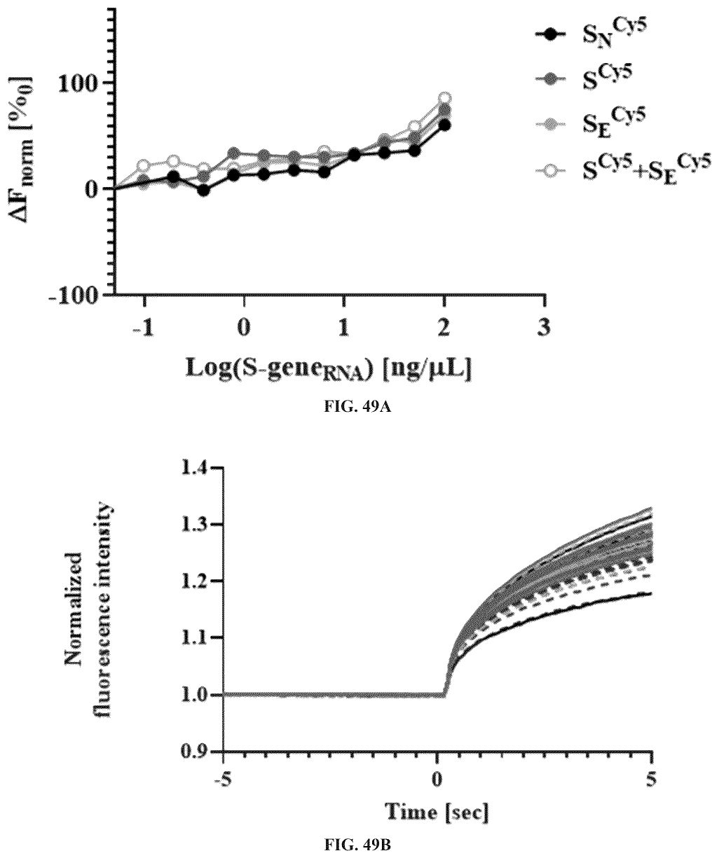

As shown in FIGS. 49A and 49B, for all of the tested probes, increasing RNA concentrations were associated with an increased signal (similar for all of the probes), as determined using the blue wavelength acquisition mode, which detects Dox fluorescence.

These results indicate that Dox interacts with RNA in a manner which depends on RNA concentration, regardless of which probe is present.

In the presence of 100 μM Dox, the flux of both S^{Cy5}+S_{E}^{Cy5} and S^{Cy5} alone (as determined using red acquisition mode) was towards the heating zone with increasing concentrations of S-gene; whereas the flux of S_{E}^{Cy5} alone was directed away from the heating zone (and with a weaker signal response than S^{Cy5} alone or S^{Cy5}+S_{E}^{Cy5}).

As shown in FIG. 50, in the presence of 100 μM Dox, the flux of S^{Cy5} alone was generally directed towards the heating zone to a greater extent than the flux of S^{Cy5}+S_{E}^{Cy5}, which is consistent with the flux of S_{E}^{Cy5} alone being directed away from the heating zone. As further shown therein, the combination of S^{Cy5}+S_{E}^{Cy5} resulted in a weaker signal than did S^{Cy5} alone in the presence of high RNA concentrations, whereas in the presence of low RNA concentrations the S^{Cy5}+S_{E}^{Cy5} response was more sensitive relative to S^{Cy5} or S_{E}^{Cy5} alone.

As shown in FIGS. 50 and 51A, the signal of S_{N}^{Cy5} was not affected by RNA concentration.

These results indicate that the probe-binding location on the target gene not only affects detection sensitivity (as discussed in Example 4), but also controls the effect of intercalating agents such as Dox on the response to the applied temperature gradient. Initial florescence values at low RNA concentrations were within 10% variability for the Cy5 labelled probes, and were therefore used for further analysis. In order to evaluate the analytical sensitivity in the linear range, the signals were fitted to a linear regression model and the slopes were compared.

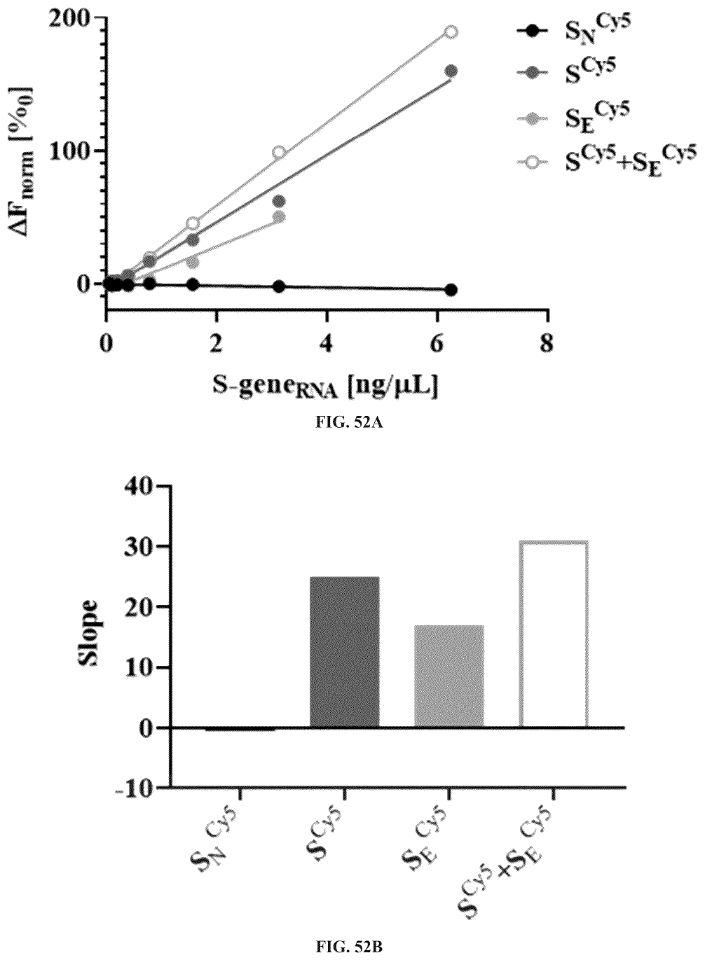

As shown in FIGS. 52A and 52B, the combination of S^{Cy5} and S_{E}^{Cy5} exhibited a greater dependence of signal on S-gene RNA concentration (upon hybridization with S-gene RNA) than did either S^{Cy5} or S_{E}^{Cy5} alone, indicating that the combination of S^{Cy5} and S_{E}^{Cy5} provides greater detection sensitivity

These results suggest that the use of a combination of probes and the post-hybridization reaction with Dox create a synergistic effect in enhancing detection sensitivity.

In order to evaluate the enhanced sensitivity more accurately, S-gene RNA was hybridized with S^{Cy5} or with a combination of S^{Cy5} and S_{E}^{Cy5}, in the absence and presence of 100 μM Dox, and the linear regression models were determined and compared.

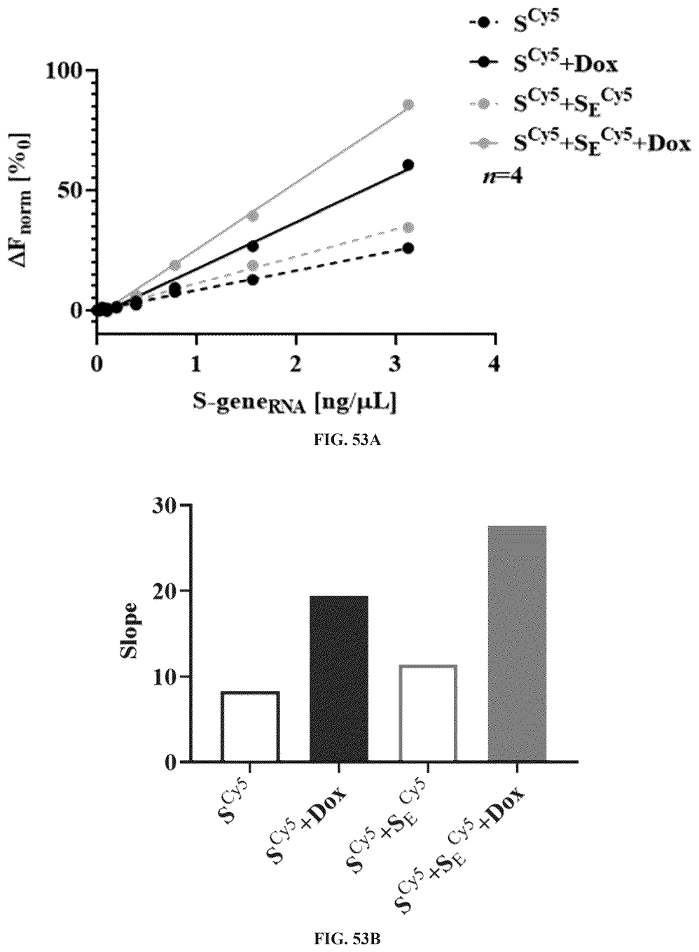

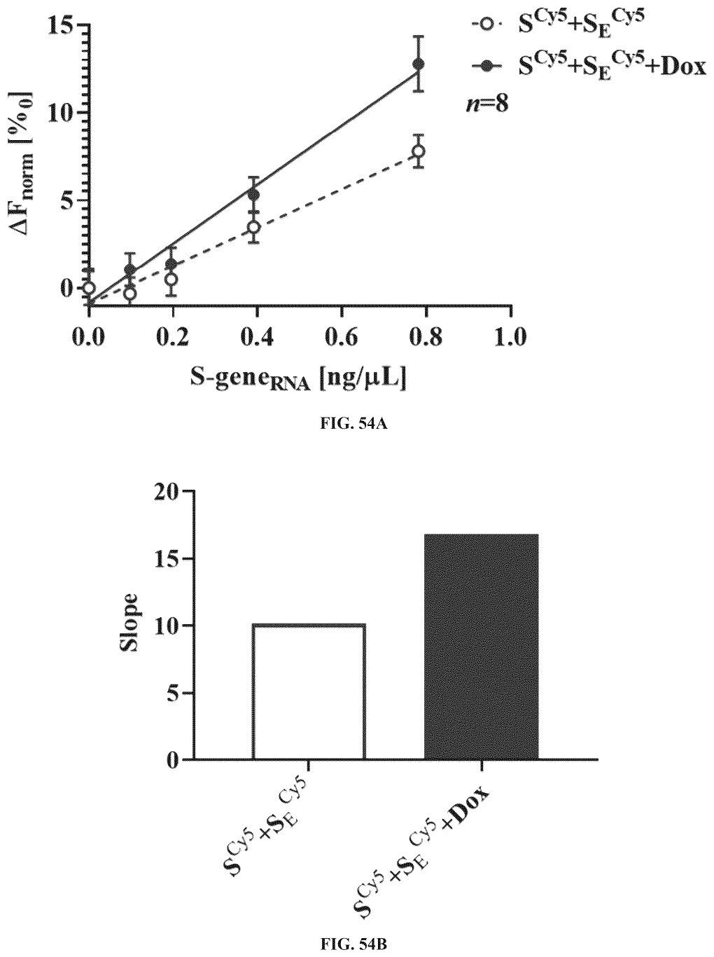

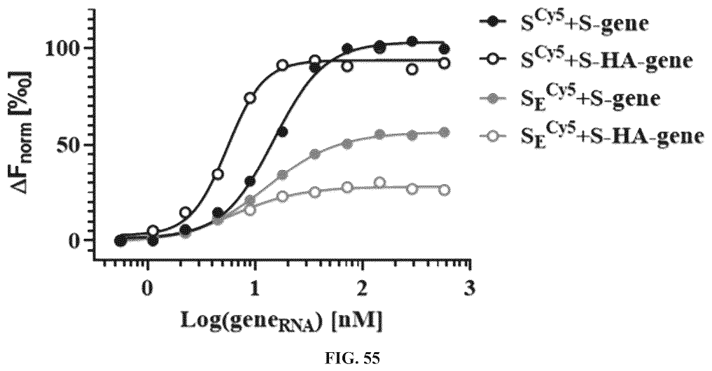

As shown in FIGS. 53A and 53B, both S^{Cy5} alone and the combination of S^{Cy5} and S_{E}^{Cy5} probes exhibited a greater dependence of signal on S-gene RNA concentration when combined with post-hybridization with Dox than without Dox, with the combination of S^{Cy5} and S_{E}^{Cy5} probes and post-hybridization with Dox resulting in the greatest dependence of signal on S-gene RNA concentration.

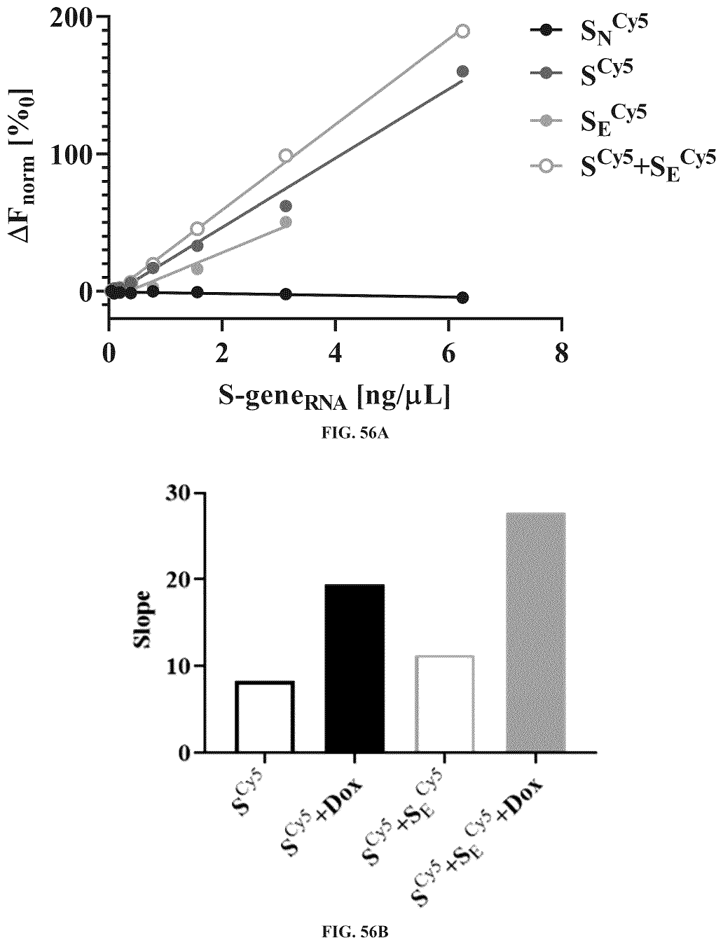

In order to evaluate sensitivity, linear regression models were generated for each incubation type. The limit of detection value (LOD_{95%}) represents the lowest amount that can be detected in a probability of 95% confidence. In addition to the previously described improvements in analytical sensitivity (as defined hereinabove), accurate values for LOD_{95%} were further determined. In order to achieve this goal, increasing concentrations of S-gene were hybridized with the combination of S^{Cy5} and S_{E}^{Cy5} in the absence and presence of 100 μM Dox, and linear regression analysis was performed, as shown in FIGS. 54A and 54B. The LOD_{95%} was calculated according to FDA guidelines using the formula: 3.3S_{y}/a, wherein S_{y} is the standard deviation of the regression and a is the linear regression slope; whereas the limit of quantification (LOQ) was calculated according to the formula: 10S_{y}/a. The LOD_{95%} value was determined to be 0.257, and the LOQ was determined to be 0.781.

These results indicate that the synergism between the use of multiple probes and the use of Dox enhances the analytical sensitivity, LOD_{95%}, and the ability to quantify the amount of target RNA.

### Example 7

### Effect of Target Gene Length and Probe Binding Location on RNA Detection Sensitivity

In the experiments described hereinabove, a single-strand RNA of about 1 kilobase was used as a target gene, whereas the SARS-CoV2 genome is about 30 kilobases, which may result in a stronger effect upon binding events with probes. In order to elucidate the effect of target gene length on detection sensitivity, an artificial gene was created by fusing the S-gene to HA-gene (each being about 1000 bases in length) to form an artificial gene of about 2000 bases in length (“S-HA gene”). As a consequence, the S_{E}^{Cy5} binding site was no longer at the edge of the gene, such that the effect of probe binding location could be further explored. S^{Cy5} and S_{E}^{Cy5} probes were incubated separately with increasing concentrations of S-gene or S-HA gene and signals were recorded using the red wavelength acquisition mode.

As shown in FIG. 55, the S^{Cy5} probe exhibited higher affinity towards S-HA gene than to S-gene, and the signal response (ΔFₙₒᵣₘ [‰] values) for S-HA gene was greater at low RNA levels than that for S-gene; whereas maximum responses did not differ between the S-HA and S-genes.

As further shown therein, S_{E}^{Cy5} probe resulted in similar signal responses for S-gene and S-HA gene at low RNA concentrations, whereas the maximum signal response was considerably stronger in the presence of S-gene than in the presence of S-HA.

In order to evaluate detection sensitivity, the signal responses at low RNA levels were fitted to linear regression models and the slopes were compared.

As shown in FIGS. 56A and 56B, the signal response of S^{Cy5} towards S-HA-gene exhibited considerably greater sensitivity than towards S-gene.

These results indicate that increased target gene length can enhance sensitivity. In addition, the considerable difference in maximum signal response, which was observed only for S_{E}^{Cy5}, suggests that the binding site location on the target gene can affect the signal response, in addition to target gene length. Moreover, the successful detection of an artificial gene demonstrates the versatility of the method described herein, and its potential ability to adapt to new mutations and/or health threats.

### Example 8

### Detection of Bacterial Ribosomal RNA

Bacterial ribosomal RNA (rRNA) is the most abundant form of RNA and each bacterium holds multiple copies of it. Detection of bacterial rRNA may therefore allow for sensitive detection of low numbers of bacteria. In addition, the amount of rRNA could indicate the number of bacteria present in a sample; and specific strain-specific regions could provide data on bacterial identification. In bacteria, one rRNA gene codes for all three rRNA types: 16S, 23S and 5S.

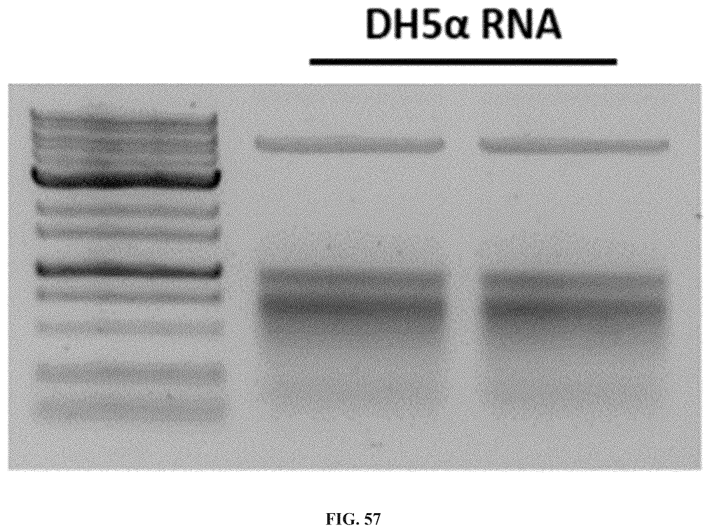

In order to evaluate the applicability of the method described herein for bacterial identification, seven Cy5-labeled probes were designed to target 16S and 23S rRNA. The structures and properties of the probes are summarized in Table 4 below. Control RNA was extracted from E. coli DH5α strain, as shown in FIG. 57.

TABLE 4
Structure and physical properties of exemplary
DNA probes complementary to E. coli 16S rRNA

| | DNA | Fluorescent | Length | | |
|---|---|---|---|---|---|
| Name | sequence | dye | (nucleotides) | Tm | GC % |
| 16S₁^{Cy5} | SEQ ID | Cy5 (red) | 42 | 89° C. | 69% |
| | NO: 9 | | | | |
| 16S₂^{Cy5} | SEQ ID | Cy5 (red) | 44 | 88° C. | 68% |
| | NO: 10 | | | | |
| 16S₃^{Cy5} | SEQ ID | Cy5 (red) | 49 | 87° C. | 63% |
| | NO: 11 | | | | |
| 16S₄^{Cy5} | SEQ ID | Cy5 (red) | 36 | 87° C. | 69% |
| | NO: 12 | | | | |
| 16S₅^{Cy5} | SEQ ID | Cy5 (red) | 31 | 86° C. | 71% |
| | NO: 13 | | | | |
| 16S₆^{Cy5} | SEQ ID | Cy5 (red) | 42 | 86° C. | 64% |
| | NO: 14 | | | | |
| 16S₇^{Cy5} | SEQ ID | Cy5 (red) | 45 | 85° C. | 62% |
| | NO: 15 | | | | |

In order to determine the optimal hybridization temperature, one of the designed probes (16S₁^{Cy5}) was incubated with increasing concentrations of bacterial RNA at different temperatures for 10 minutes. MST measurements were then performed using the red wavelength acquisition mode.

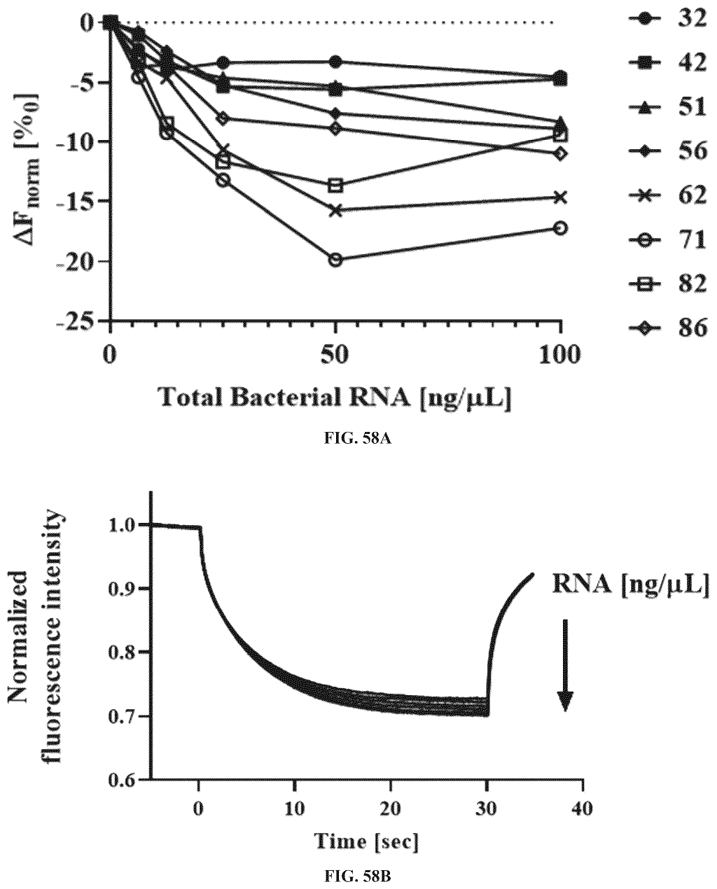

As shown in FIGS. 58A and 58B, the observed signal was most responsive to target RNA concentration when 16S₁^{Cy5} was incubated at 71° C., wherein the MST curves exhibited a decrease in fluorescence with increasing concentration of RNA, indicating that the 16S₁^{Cy5} probe migrated away from the heating zone when bound to its target to a greater extent than in its free form.

In order to assess the effect of human RNA presence and Dox post-hybridization on signal response, 16S₁^{Cy5} was incubated with increasing concentrations of bacterial or human total RNA at 71° C. for 2 hours. Equal amounts were then taken for post-hybridization incubation with Dox or hybridization buffer as a control.

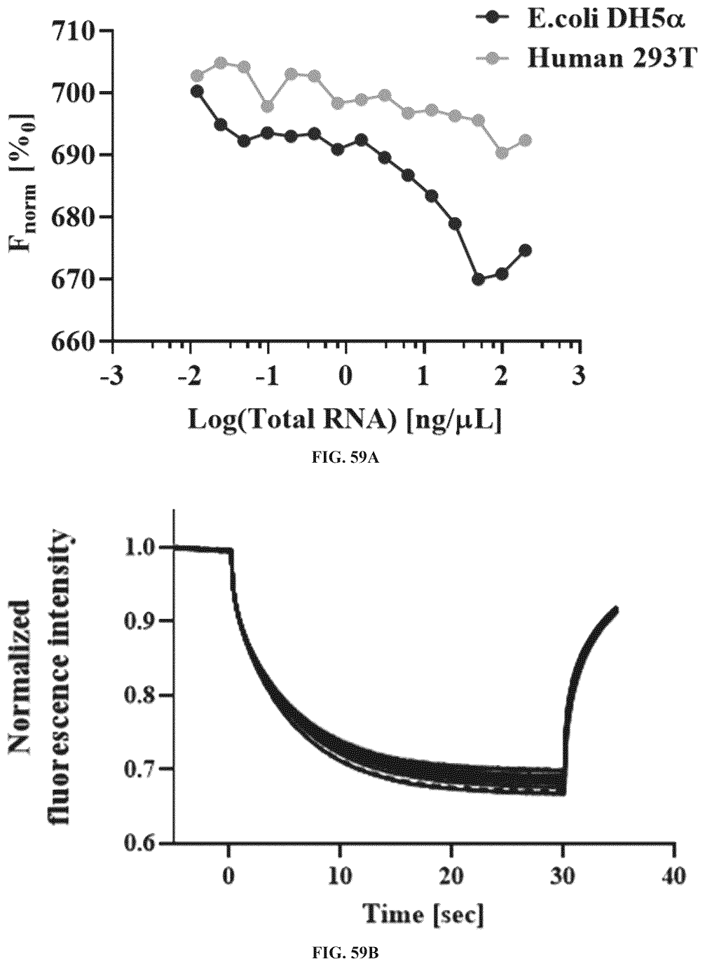

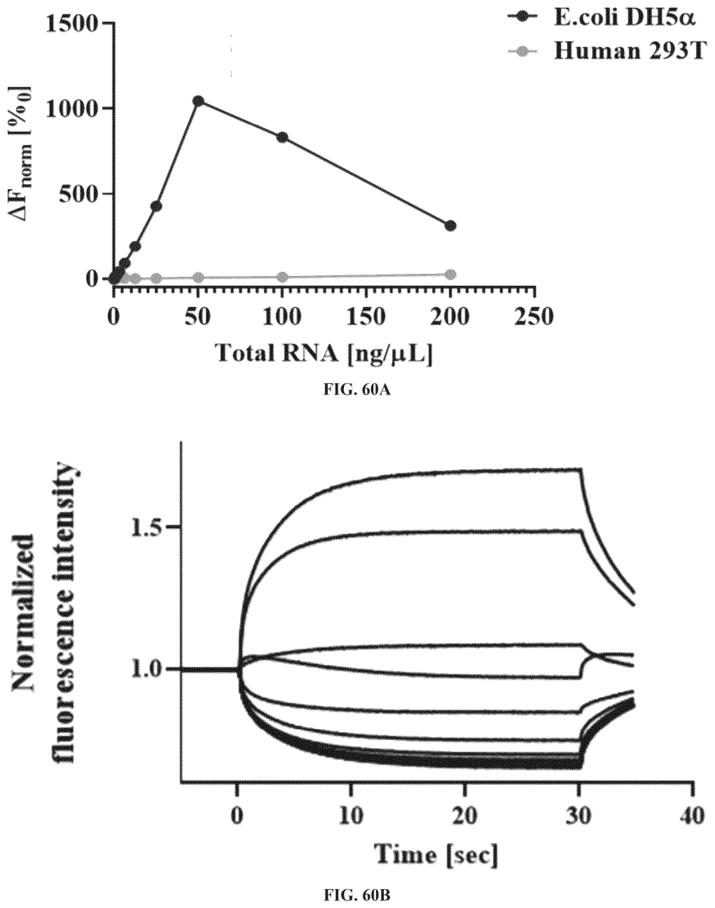

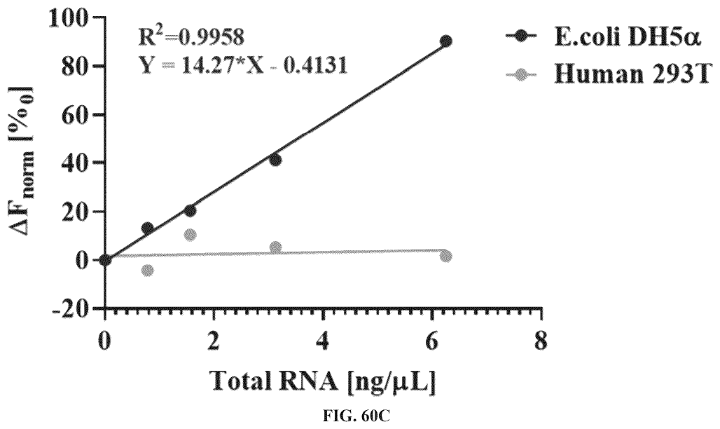

As shown in FIGS. 59A-60C, the observed signals in the absence (FIGS. 59A and 59B) or presence (FIG. 60A-60C) of Dox exhibited a clear dependence on the concentration of bacterial RNA but not on the concentration of human RNA (as determined using the red wavelength acquisition mode). In addition, the presence of Dox changed ΔFₙₒᵣₘ values from negative to positive (with increasing concentration of bacterial RNA but not human RNA), indicating that Dox alters the thermophoretic migration of the 16S₁^{Cy5}:RNA hybrid, causing it to migrate towards the heating zone.

As shown in FIG. 60A, when RNA levels were above 50 ng/μl, the effect of Dox on the thermophoretic migration decreased, apparently due to competitive binding with the DNA:RNA hybrid.

As shown in FIG. 60C, the signal in the presence of Dox strongly fit (R²=0.9958) a linear regression model for low concentrations of RNA (up to 6.25 ng/μl), whereas the signals in the absence of Dox did not fit a linear regression model in such a manner (data not shown).

These results indicate that the use of Dox enhanced the dose-dependent nature of the signal.

In order to compare signal sensitivity in the presence and absence of Dox, ΔFₙₒᵣₘ[‰] values of human RNA were subtracted (ΔΔFₙₒᵣₘ), and the ratios of ΔΔFₙₒᵣₘ[‰] were compared.

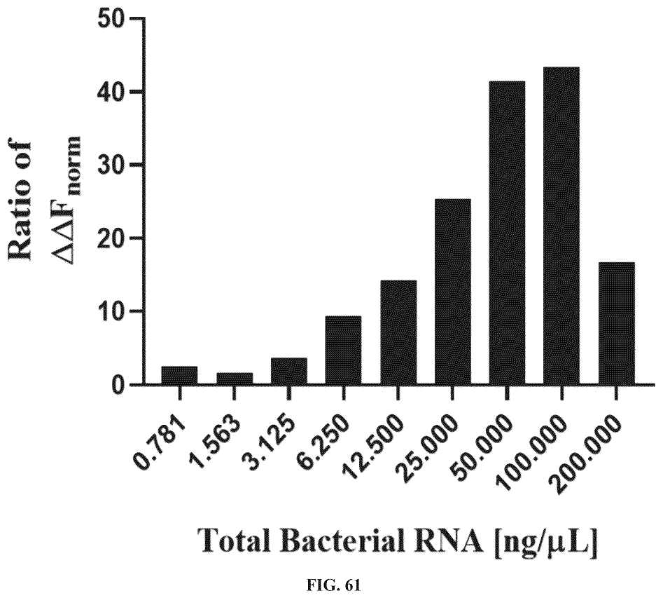

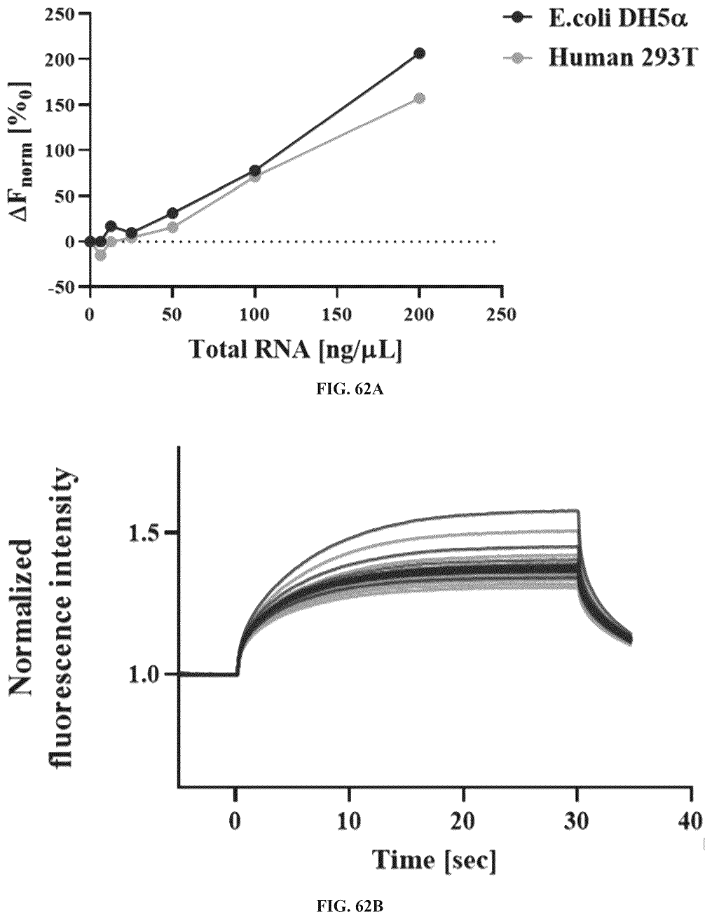

As shown in FIG. 61, the presence of Dox resulted in increased signal sensitivity which facilitates quantification of 16S rRNA, which was less reliable in the absence of Dox.

As shown in FIGS. 62A and 62B, the Dox signal observed using the blue wavelength acquisition was dependent on the concentrations of both human RNA and bacterial RNA; whereas in the presence of 200 ng/μl RNA, the signal response was stronger for bacterial RNA. As further shown therein, MST curves for RNA concentrations above 50 ng/μl indicated a more efficient migration of Dox into the heating zone in the presence of such RNA concentrations.

These results indicate that Dox MST signals (e.g., of one-point measurements relative to control) can be inspected to determine if binding efficiency towards the probe-target nucleic acid hybrid is reduced due to competition with high concentrations of RNA.

In order to evaluate what time period is sufficient for hybridization, 16S₁^{Cy5} probe was incubated at 71° C. with various concentrations of bacterial RNA for different hybridization periods ranging from 5 to 120 minutes in the presence of Dox.

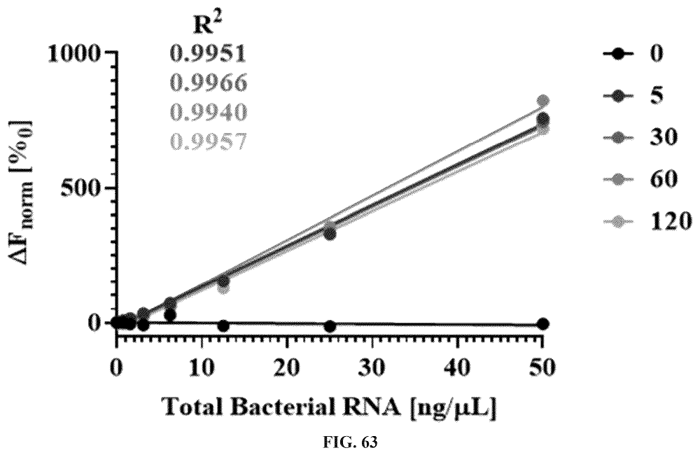

As shown in FIG. 63, a similar dose-depended increase in signal was observed at all tested hybridization periods in the range of 5 to 120 minutes (using the red wavelength acquisition mode), whereas no change in signal was observed without hybridization (i.e., a hybridization period of 0).

These results indicate that a time period of less than 5 minutes may be sufficient for hybridization.

An additional 6 probes for 16S rRNA were tested under conditions such as described hereinabove for 16S₁^{Cy5}, in the presence of Dox.

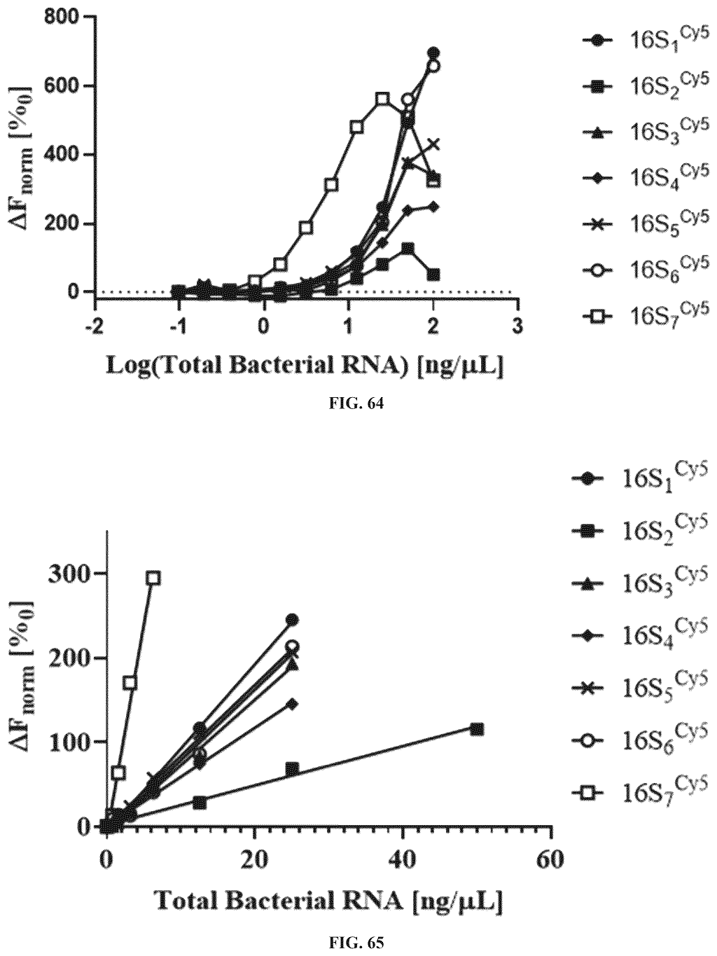

As shown in FIGS. 64 and 65, a dose-depended increase in signal was observed for all tested probes (using the red wavelength acquisition mode), with the 16S₇^{Cy5} probe being significantly more sensitive to target RNA concentration than the other tested probes, the sensitivity rank of the tested probes being 7>1>5>6>3>4>2.

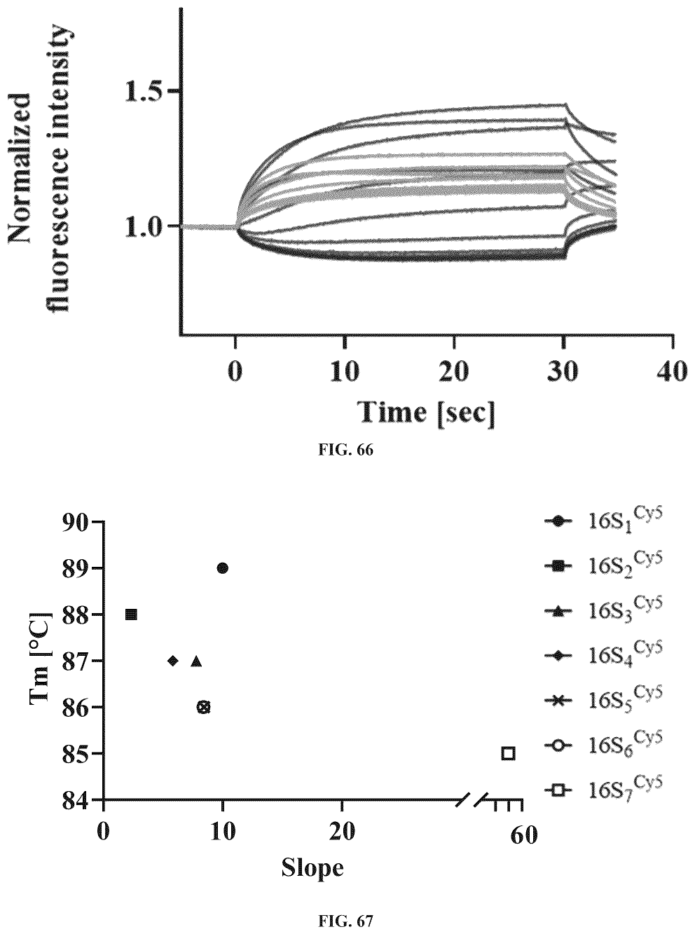

As shown in FIG. 66, the MST curves indicated that 16S₇^{Cy5} migrated towards the heating zone in the presence of increasing concentrations of bacterial RNA, but away from the heating zone in the absence of bacterial RNA; whereas 16S₂^{Cy5} migrated towards the heating zone both in the presence of bacterial RNA (albeit less efficiently than 16S₇^{Cy5}) and in the absence of bacterial RNA. A similar effect was observed for 16S₄^{Cy5} (data not shown).

As shown in FIG. 67, the analytical sensitivity of the various probes, as determined by the linear regression slope, was not correlated with their Tm values.

These results indicate that although high Tm values are indicative of specificity to the target probe, other parameters affect analytical sensitivity (in the presence of Dox).

In addition, in order to investigate the effect of probe combination on detection sensitivity in the presence of Dox, the 16S₇^{Cy5} probe was combined with one or more of each of the other (16S₁^{Cy5}-16S₆^{Cy5}) probes.

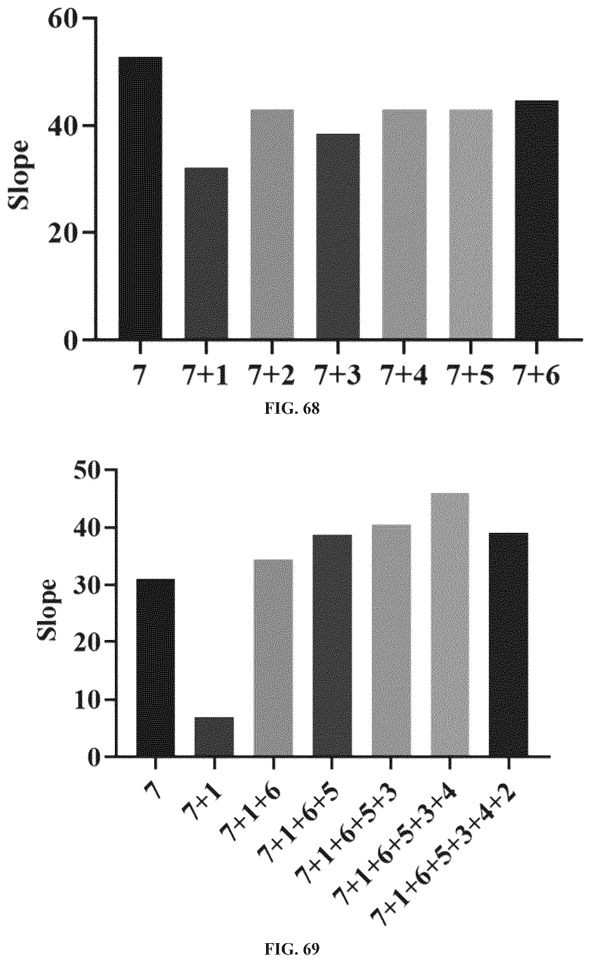

As shown in FIG. 68, each combination of 16S₇^{Cy5} with one other probe was associated with decreased signal sensitivity relative to 16S₇^{Cy5} alone.

This result suggests that the respective binding sites for 16S₇^{Cy5} and each of the other probes have a negative cooperativity effect.

As shown in FIG. 69, although combination of 16S₇^{Cy5} with 16S₁^{Cy5} resulted in reduced signal sensitivity relative to 16S₇^{Cy5} alone (as discussed hereinabove), the combination of 16S₇^{Cy5} with 16S₁^{Cy5} and at least one other probe could increase signal sensitivity relative to 16S₇^{Cy5} alone.

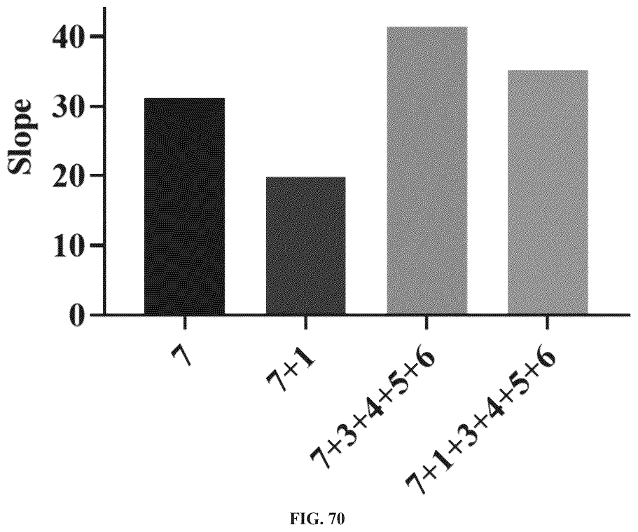

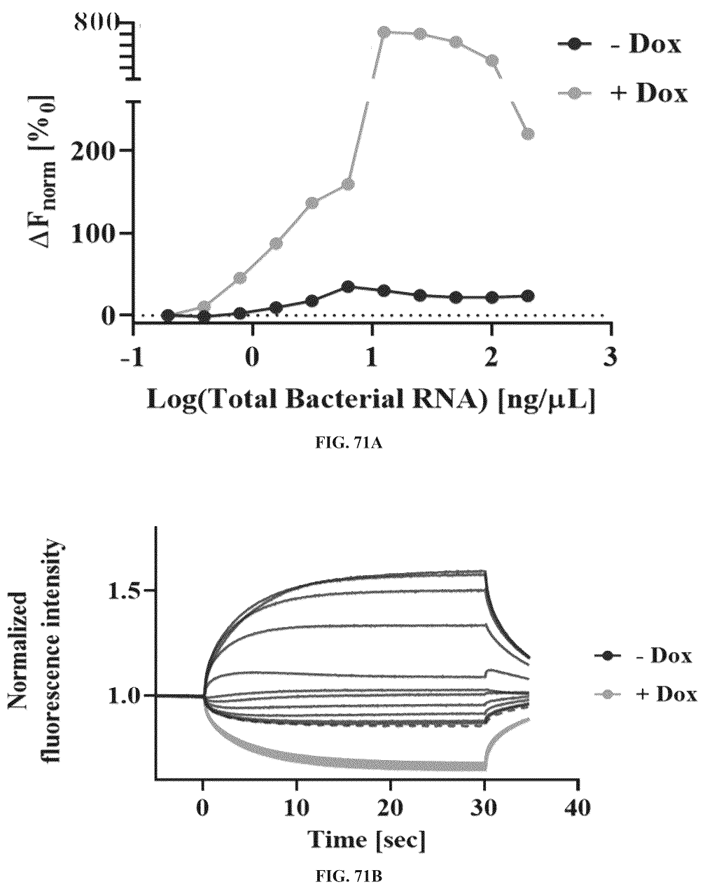

As shown in FIG. 70, the combination of probes 16S₃^{Cy5}, 16S₄^{Cy5}, 16S₅^{Cy5} and 16S₆^{Cy5} with 16S₇^{Cy5} resulted in greater signal sensitivity when 16S₁^{Cy5} was absent than when 16S₁^{Cy5} was also included.

The above results indicate that combinations of probes may increase signal sensitivity, but do not necessarily do so.

In view of the above results, a probe combination was selected which included 16S₃^{Cy5}, 16S₅^{Cy5}, 16S₆^{Cy5} and 16S₇^{Cy5}; but not 16S₁^{Cy5} or 16S₂^{Cy5} (in view of the results shown in FIG. 70), and not 16S₄^{Cy5}, in view of the migration of this probe towards the heating zone even in the absence of RNA, similarly to 16S₂^{Cy5} (as discussed hereinabove). The selected probe combination was hybridized with increasing concentrations of bacterial RNA, in the absence or presence of Dox post-hybridization.

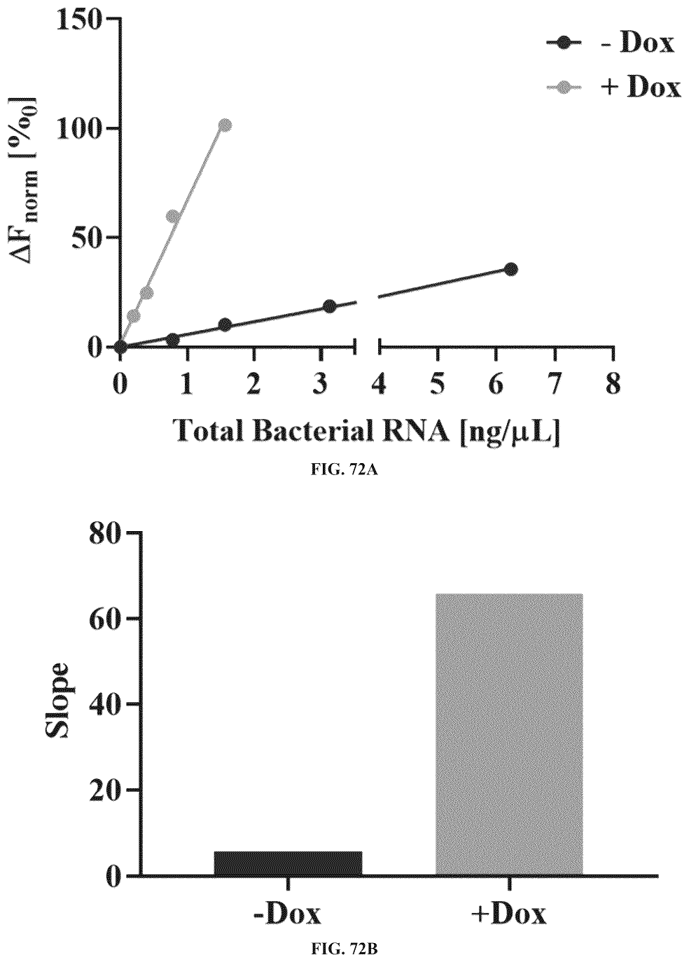

As shown in FIGS. 71A to 72B, the presence of Dox enhanced signal sensitivity to a considerable extent, such that total RNA at a concentration as low as 0.12 ng/μl could be detected.

Taken together, the above results indicate that the presence of bacteria could be quantitatively determined using a method such as described herein, and may facilitate monitoring of bacterial growth and/or identification of bacteria based on rRNA. These results further exemplify a calibration process for a new target nucleic acid (e.g., for determining optimal probe, hybridization temperature, hybridization time, and/or use of an intercalating agent such as Dox).

### Example 9

### Detection of Additional Bacterial RNA Targets

One or more probes complementary to bacterial RNA is designed, according to procedures described hereinabove, except that the bacterial RNA is 23S rRNA or RNA associated with an antibiotic resistance gene, optionally New Delhi metallo-beta-lactamase 1 antibiotic resistance gene (NDM-1) of pathogenic E. coli. Optional hybridization temperature, hybridization time, and/or use of an intercalating agent such as Dox is determined, according to procures such as described in Example 8.

Optionally, sensitivity is enhanced by using two or more probes concomitantly, for example, a probe targeting 16S rRNA as described in Example 8 and a probe targeting 23S rRNA as described herein, e.g., wherein both probes bind different targets but have the same fluorescent tag (which may improve detection sensitivity by enhancing Cy5 binding capacity).

### Example 10

### Bacterial Infections Diagnosis by Targeting 16S Ribosomal RNA Using MST

Rapid identification of bacterial infections is crucial for providing a proper medical treatment. To demonstrate the applicability of the current method for bacterial identification, seven Cy5 labeled probes (16S₁₋₇^{Cy5}) were designed to target bacterial specific 16S rRNA. Sample preparation protocol was developed for bacterial MST experiments and total RNA was extracted from E. coli DH5α or BL-21 strains.

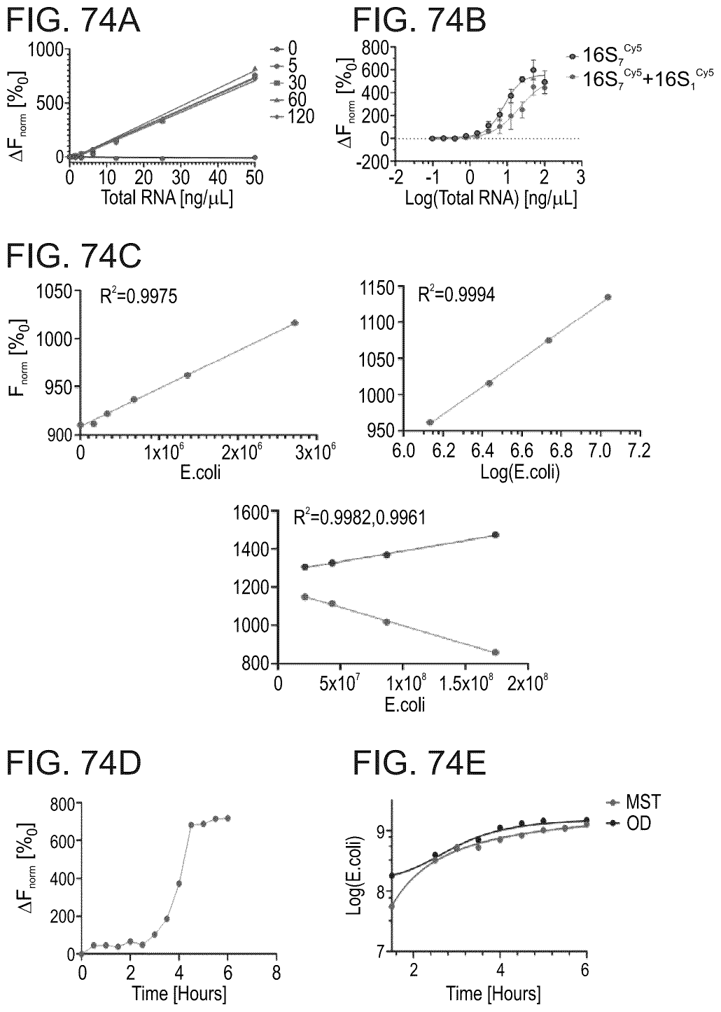

Hybridization with uniformly labeled probes and post hybridization with Dox improved detection sensitivity. As demonstrated for 16S₁^{Cy5}, the sufficient time for hybridization at 71° C. was determined to be as early as 5 min after RNA extraction (see, FIG. 74A).

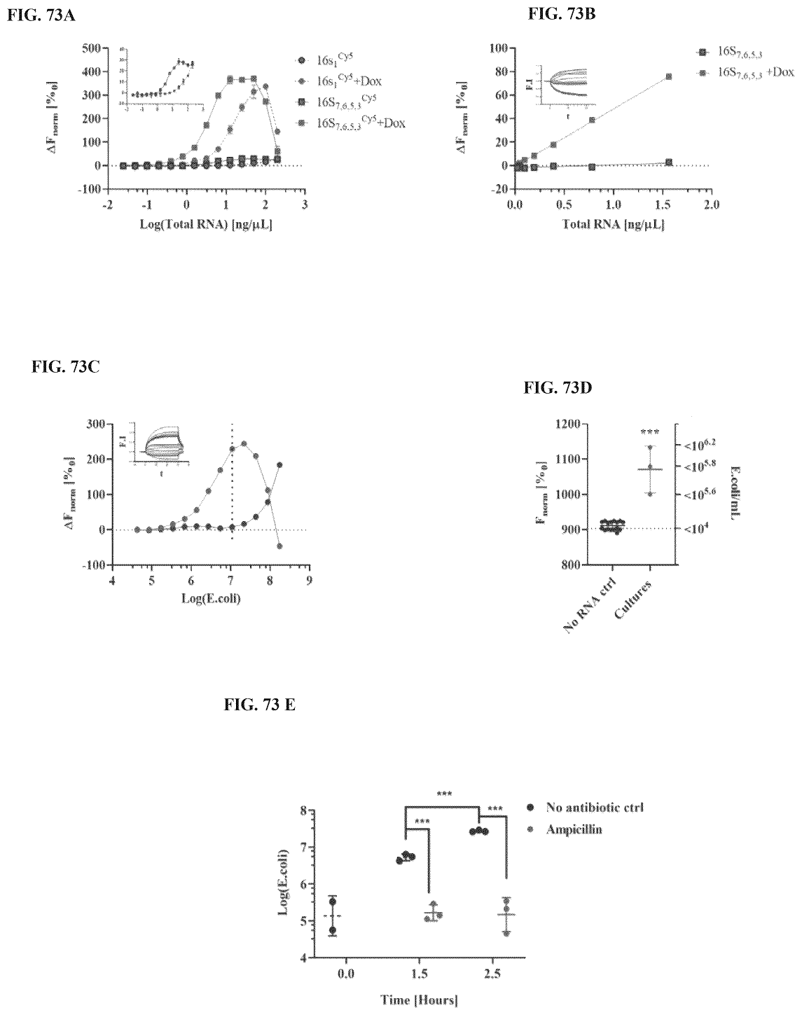

FIGS. 73A-E present bacterial infections diagnosis by targeting 16S ribosomal RNA using MST; (FIG. 73A) The dose-dependent generated signals of 16S₁^{Cy5} probe and 16S_{7,6,5,3}^{Cy5} probe mix hybridized with increasing concentrations of total extracted RNA, followed by post-hybridization with 100 μM Dox or hybridization buffer as control; Measurements recorded using the red wavelength acquisition mode; Magnification of the responses without Dox are shown in the small graph; Error bars representing SD of different replicates n=2; (FIG. 73B) The linear regression model of (FIG. 73A); All MST curves of 16S_{7,6,5,3}^{Cy5} probe mix followed by post-hybridization incubation with 100 μM Dox (grey) or hybridization buffer as control (black) are shown in the upper graph; Error bars representing SD of different replicates n=2; (FIG. 73C) Representative standard curve of the generated signals of 16S_{7,6,5,3}^{Cy5} probe mix hybridized with increasing concentrations of total extracted RNA from 10⁸ E. coli bacteria, followed by post-hybridization incubation with 100 μM Dox; The dashed line represents the starting of the competition between the DNA:RNA hybrid and free RNA on Dox, and characterized by Dox (black line) signal increase and Cy5 (grey line) signal decrease; MST curves are shown in the upper graph. (FIG. 73D) Bacterial load determination in 10 mL of natural urine samples spiked with pathogenic E. coli strains; The strains derived from three different human patients (n=3); No RNA control (n=16); Error bars representing SD of different samples; (FIG. 73E) Antimicrobial susceptibility testing (AST) of 5*10⁵ E. coli bacterial cultures grown for 90-150 minutes in the presence of 12.5 mg/mL ampicillin antibiotic. t₀ n=2, t_{1.5} and t_{2.5} n=3; Error bars representing SD of independent cultures; *** p value<0.001 according to student t-test; Error bars representing SD of different cell cultures; F.I-Fluorescence intensity, t-Time in MST graphs.

FIGS. 74A-E present bacterial infections diagnosis by targeting 16S ribosomal RNA using MST; (FIG. 74A) The effect of hybridization time at 71° C. on the dose-dependent generated signals of 16S₁^{Cy5} probe hybridized with increasing concentrations of total bacterial RNA followed by post-hybridization with 100 μM Dox; signals recorded using the red wavelength acquisition mode; (FIG. 74B) The dose-dependent generated signals of 16S₇^{Cy5} in the presence and absence of 16S₁^{Cy5} hybridized with increasing concentrations of total RNA, followed by post-hybridization with 100 μM Dox; Error bars describing SEM of two independent experiments; (FIG. 74C); The different linear regression models of FIG. 74C which are used for bacterial load determination; (FIGS. 74D-E) Two independent growth curves of E. coli; (FIG. 74D) Showing the increase in signal as represented by ΔFₙₒᵣₘ [‰] values; Growth started from 10⁵ bacteria (FIG. 74E) extrapolated bacteria values; Growth started from 10⁸ bacteria and optical density (O.D) measurements were preformed prior RNA extraction.

During system calibration, 16S₇^{Cy5} and 16S₁^{Cy5} combination showed to be less sensitive then 16S₇^{Cy5} alone (FIG. 74B). This indicating that although the combination of probes can improve detection it is not necessarily additive effect and needs to be determined experimentally. The response of the chosen combination 16S_{7,6,5,3}^{Cy5} is shown compered to 16S₁^{Cy5} alone in the absence or presence of post-hybridization with Dox (FIG. 73A). 16S_{7,6,5,3}^{Cy5} showed improved detection sensitivity with and without Dox compered to 16S₁^{Cy5} where the response of 16S₁^{Cy5} without Dox showed neglectable signals. The effect of Dox on the linear response of 16S_{7,6,5,3}^{Cy5} is demonstrated in FIG. 73B.

Determining the amount of bacterial load in urine samples is essential for identifying positive samples. A representative standard curve is shown in FIG. 73C and its 3 linear ranges for quantification are shown in FIG. 74C. To determine the current range for quantification, Cy5 signals needs to be inspected relative to Dox, where above 107 bacteria, Cy5 signals decreases while Dox increases. To demonstrate urine samples diagnosis, pathogenic E. Coli colonies were taken from 3 different positive samples and spiked into natural urine ones. After, MST diagnosis procedure was performed where the concentration of E. coli bacteria was successfully determined relative to no RNA control (FIG. 73D). Moreover, the probe combination showed to be useful for detecting various sub-strains including clinically relevant ones.

16S rRNA amount is proportional to bacteria number and therefore can be used to monitor growth. Bacterial growth was examined from starting number of 10⁵ bacteria, the current LOD of the MST diagnosis method. The time dependent signal responses were corresponding to the accepted bacterial growth phases (FIG. 74D). To compere the MST method to the commonly used optical density (O.D) measurements in microbiology laboratories, the experiment was repeated with the LOD of O.D, 10⁸ E. coli. O.D and subsequent MST measurements were performed for the same cultures where the signal responses were increased with time and bacteria number was highly positively correlated between the methods (FIG. 74E). To date, antimicrobial susceptibility testing (AST) is used to determine the resistance profile of bacteria for clinical diagnosis. At least 48 hours are required in order to establish the full resistance profile and to prescribe the proper antibiotic. Unfortunately, this long detection period results in a delay treatment which is highly correlated with mortality. Therefore, we utilized the ability of our system to detect small growth changes starting from low amount of bacteria. To do so, the growth of E. coli bacteria was monitored in the presence of Ampicillin (Amp), starting from 5*10⁵ bacteria. Growth was observed in the absence of Amp as early as 90 minutes, while no growth was observed in its presence at both time periods (FIG. 73E). This data indicates that AST can be determined in a time widow of less than 1.5 hours. Thus, providing a fast and accurate method to detect growth and antibiotic resistance.

### Materials and Methods

Some of the methods have been presented with some variations hereinabove.

DNA probes conjugated to Cyanine 5 (Cy5) or ATTO 488 dye were obtained from Metabion and formamide and doxycycline were obtained from Sigma. Phosphate buffered saline (PBS, Biological Industries, IL) buffer contains 500 mM phosphate and 1500 mM NaCl (referred to herein as “X10”) Saline sodium citrate (SSC, BioLabs) buffer contains 600 mM trisodium citrate and 3 M NaCl pH 7. Tween-20 100% and Glycerol were purchased from Biolabs. Tris and kanamycin were obtained from Sigma. TRIzol™ reagent and Doxorubicin HCl were purchased from Invitrogen and Tzamal D-Chem laboratories, respectively. Ampilicin and Bacto tryptone (CN-211705) were purchased from Enzo and Gibco, respectively. Bacto yeast (CN-212750) extract and Bacto agar (CN-214010) were purchased from BD.

### Cell Culture

U2 osteosarcoma (OS) cells stably expressing inducible YFP-24×MS2 were grown as previously described with some modifications in low glucose Dulbecco's modified Eagle's medium (DMEM) containing 10% fetal bovine serum (FBS) and 1% penicillin-streptomycin. Transcription was induced with 1 μg/ml doxycycline overnight and after, cells were harvested on cold ice with PBS X1 for further RNA extraction. HEK293T cells were grown as previously described in high glucose DMEM containing 10% FBS, 1% penicillin-streptomycin and 1% glutamine supplementation. Cells were harvested in TRIzol reagent for RNA extraction.

### Cloning

S and HA, gene fragments, 1089 bp and 1111 bp respectively, were cloned into pcDNA3.1+ expression vector, using BamHI+EcoRI and BamHI+XhoI restriction enzymes, respectively. Merged plasmid containing both S-gene and HA-gene was cloned using EcoRI+XhoI restriction enzymes, when S-gene is on the 5′ region and the HA on the 3′. The RNA transcription was conducted using T7 in-vitro transcription kit (Promega). The Gene-S and HA sequences were obtained from 2019 nCoV (SARS CoV2) whole genome sequence (GenBank: MN908947 and GenBank: CY249803.1 respectively).

### Probe Design

Target sequences (of SARS CoV2 S-gene, N1H1 HA-gene, human 18S rRNA and 16S rRNA) were taken from NCBI GenBank and converted to the reverse complement sequence. In order to achieve maximal melting temperature (Tm), GC-rich regions, an indication of a strong binding potential, were detected in each sequence, based upon which the probes were designed. Sequences of 20-35 nucleotides in length were uploaded to NEB Tm calculator to determine GC content and Tm. In addition, physical constants of the oligonucleotides such as mass, charge, GC content, Tₘ value and self-complementary potential were analyzed using the OligoCalc oligonucleotide properties calculator. Potential probe sequences with self-complementarity of a minimum of 5 bp required for self-dimerization and/or a minimum of 4 bp required for hairpin formation were filtered out. To reduce binding to off targets and to enhance specificity of the probes, the sequences were aligned in NCBI BLAST versus standard database and the human transcriptome database. Potential probe sequences with any partial match to other pathogens and/or high similarity (more than 60% identity) to a sequence in the human transcriptome were filtered out.

### In-Vitro Transcription

pcDNA3.1+ containing about 1000 bp of either S-gene or HA-gene was linearized with either EcoRI or XhoI restriction enzyme, respectively, and submitted to an in-vitro transcription process using a T7 RiboMAX™ kit (Promega) and cleared from DNA using DNase for 15 minutes at 37° C.

### RNA Extraction

RNA was extracted from samples using the guanidinium thiocyanate phenol-chloroform extraction technique, using TRIzol™ reagent. 1 ml of TRIzol™ reagent was added to a cell pellet or RNA solution after in vitro transcription. The samples were then incubated for 5 minutes at room temperature. For phase separation, 0.2 ml of chloroform was added per 1 ml of TRIzol™ reagent, and the samples were gently inverted and incubated at room temperature for 3 minutes. The samples were then centrifuged at 12,000×g for 15 minutes at 4° C. Following centrifugation, the mixture separates into a lower (pink) phenol-chloroform phase, an interphase, and an upper (transparent) aqueous phase, wherein RNA remains exclusively in the aqueous phase. The upper phase was transferred into a fresh tube without disturbing the interphase. For RNA precipitation, 0.5 ml of isopropyl alcohol was added to the tube per 400 μl of the aqueous phase. The samples were incubated at room temperature for 10 minutes and centrifuged at 12,000×g for 10 minutes at 4° C. The supernatant was discarded completely, and the RNA pellet was washed twice with 1 ml of 75% ethanol, mixed by gently inverting the tube, and centrifuged at 12,000×g for 10 minutes at 4° C. After ethanol removal the samples were left to dry in the chemical hood with open tubes for 15-20 minutes.

For bacterial RNA extraction, DH5α E. coli cells transfected with pcDNA3.1+ plasmid were grown overnight in LB medium containing 100 μg/ml ampicillin. The concentration of bacterial cells was determined according to OD_{600 nm}, and about 7*10⁸ cells were taken for RNA extraction. 1 volume of bacterial culture was mixed with 2 volumes of RNAprotect® Bacteria Reagent (QIAGEN), vortexed for 5 seconds and incubated for 5 minutes at room temperature. The sample was then centrifuged for 10 minutes at 5,000×g at room temperature. After discarding supernatant, the pellet was resuspended in 100 μl TE buffer (10 mM Tris-HCl, 1 mM EDTA, pH 8) containing 1 mg/ml lysozyme. The samples were vortexed for 5 seconds, and then incubated at room temperature for 10 minutes with vortexing every 2 minutes. 1 ml TRIzol™ reagent was added to the sample. The sample was incubated at room temperature for 5 minutes, and then RNA extraction was carried out as described previously.

### Hybridization Setup

For RNA titration measurements, the extracted RNA solution was diluted to X2 and a serial dilution was prepared in water. Hybridization solutions were prepared as described herein, were X1 solution consisted of SSC buffer X1, 0.05% TWEEN™ 20 surfactant, and 1% formamide unless indicated otherwise. Fluorescently labeled probes were diluted from a 100 μM stock to 1:2400 in hybridization solution X2, denatured for 5 minutes at 100° C. and then incubated on ice for 5 minutes. If a mixture of uniformly labeled probes was prepared, the probes were diluted to a 1:1200 dilution in water and then diluted to 1:2400 with hybridization solution X4 (1:1/number of probes) to create equal molarity of the fluorescent dye. The probe solution was combined with the titration series in a 1:1 (v/v) ratio (10 μL total volume) and allowed to hybridize at a defined temperature for a given time as described. If post-hybridization with intercalating agents (ICA), Doxorubicin or Midori™ Green, was performed, the samples were combined with ICA X5 solution 4:1 (v:v) ratio, 8 μL and 2 μL respectively. For cell based measurements, after extraction, the RNA pellet was solubilized and hybridized with a specific fluorescently labeled DNA probe in a hybridization solution.

### Bacterial Growth

Kanamycin resistant E. coli bacteria, DH5α or BL-21, were maintained as frozen stocks at −80° C. in homemade L.B (Bacto tryptone 10 g/L, NaCl 10 g/L Bacto yest extract 5 g/L in autoclavable DDW) supplemented with 50% Glycerol as cryoprotective agent. Cultures were started from frozen stocks and cultivated at 37° C. overnight, at the presence of 30 μg/ml Kanamycin. After starter growth, bacteria amount was measured using optical density for further experiments.

### MST to O.D Comparison

Bacteria was grown as described in “bacterial growth” section and adjusted to 0.1 O.D for a second growing step after overnight starter. In 30 min interval 1 ml samples were taken out for O.D measurements and then combined with RNA protect reagent for RNA extraction as previously described.

### Antimicrobial Susceptibility Testing (AST)

Bacteria was grown as described in “bacterial growth” section and adjusted to 0.1 O.D for a second growing step after overnight starter. When reached 0.6 O.D, bacterial concentration adjusted to 7.143*10⁵ bacteria/ml and 0.7 ml (5*10⁵ bacteria) were divided to slit-cap Eppendorf tubes containing Ampicillin antibiotic or water as control. The tubes were placed in a temperature control thermo-mixer for further growth at 37° C. At an indicated time-point, tubes were taken out combined with RNA protect reagent for RNA extraction as previously described.

### Microscale Thermophoresis Measurements

RNA was extracted from a sample, and after extraction, the RNA pellet was solubilized and hybridized with a specific fluorescently labeled DNA probe in a hybridization solution. After hybridization, the hybridized sample was loaded into an MST capillary and inserted into a Monolith™ NT.115 red/blue MST instrument (NanoTemper Technologies GmbH, Germany). This instrument included a blue mode (493 nm excitation, 650 nm emission) and red mode (521 nm excitation, 670 nm emission) for detection, and an I.R laser (1475 nm+/−15 nm) for heating. The intensities for both fluorescence detection (LED) and IR laser are adjustable, in a range of 0%-100%, wherein the temperature gradient generated has a temperature difference of up to about 2-8° C. Thermophoretic force was induced by infrared laser after 1 second, and maintained for 2 seconds, for a total of a 3 second measurement window (measurement time may optionally be further reduced to 2 seconds using current available MST instruments) before applying the thermophoretic force, both free probe and the DNA:RNA hybrid are distributed evenly in space due to mass diffusion; therefore, fluorescence signal is constant. Upon application of a thermophoretic force, both the free probe and the DNA:RNA hybrid migrate along the thermophoretic field away from or into the heating zone. During that period, the flux is controlled by both mass and thermophoretic diffusions; therefore, a change in fluorescence is observed. fluorescent signals were quantified in the 1 second before application of a temperature gradient (F_{cold}) and between 1 and 2 seconds after application of the temperature gradient (Fₕₒₜ).

For bacterial infections-based assays, a standard curve of E. coli bacteria was prepared using a known amount of bacteria and MST signal was correlated with bacterial amount or RNA concentration. Samples were measured first using the red wavelength acquisition mode to determine the specific signal for E. coli and the blue wavelength acquisition mode to determine if Dox competition effects the red signal. If so, samples were further diluted.

### Calculations and Graphical Representation

The thermophoretic migration of a molecule is represented as Fₙₒᵣₘ[‰] values and calculated using the formula Fₕₒₜ/F_{cold}×1000=Fₙₒᵣₘ[‰], where F_{cold} is the florescence intensity before applying the force, and F_{Hot} is the florescence intensity after applying the force. 1000<Fₙₒᵣₘ[‰] indicating on flux directionality inside the measurement zone and vice versa. ΔFₙₒᵣₘ [‰] represents the change in migration flux relative to the initial state, where positive values indicating on a migration trend into the measurement area and vice versa. Fₙₒᵣₘ [‰] values were extracted as raw data and analyzed using the Prism software v.8. Each experiment was performed at least twice in independent experiments. Z factor statistical parameter was calculated according to the formula

$$Z = 1 - \frac{3 * ( σ p - σ n )}{\vert μ p - μ n \vert}$$

where p and n represents positive and negative groups respectively, σ and μ represents standard deviation and mean respectively

Sequence tables

| | SEQ | | | | Length | | |
|---|---|---|---|---|---|---|---|
| | ID | | | Fluorescent | (nucleo- | Tm | GC |
| Name | No. | DNA sequence | Target gene | dye | tides) | ° C. | % |
| S^{Cy5} | 16 | GGATTGACTAGCTA | SARS-CoV2 | Cy5 (red) | 33 | 79 | 61 |
| | | CACTACGTGCCCGC | spike gene (S- | | | | |
| | | CGAGG | gene) | | | | |
| S_{E}^{Cy5} | 17 | CAATATCACCAAGG | SARS-CoV2 | Cy5 (red) | 22 | 64 | 45 |
| | | CAATCACC | spike gene (S- | | | | |
| | | | gene) | | | | |
| S_{N}^{Cy5} | 18 | CACCATTAGTGGGT | SARS-CoV2 | Cy5 (red) | 22 | 66 | 50 |
| | | TGGAAACC | spike gene (S- | | | | |
| | | | gene) | | | | |
| HA^{ATTO488} | 19 | TGAAGCCGGCAATG | HA gene | ATTO488 | 33 | 81 | 61 |
| | | GCCCCGAATAGGCC | (H1N1 | (blue) | | | |
| | | TCTAG | influenza) | | | | |
| 18S^{ATTO488} | 20 | AGGGGCTGACCGG | 18S rRNA | ATTO 488 | 46 | 83 | 54 |
| | | GTTGGTTTTGATCT | (human) | (blue) | | | |
| | | GATAAATGCACGCA | | | | | |
| | | TCCCC | | | | | |
| 16S^{Cy5} | 21 | CCCACTCCCATGGT | 16S rRNA (E. | Cy5 (red) | 42 | 89 | 69 |
| | | GTGACGGGCGGTGT | coli) | | | | |
| | | GTACAAGGCCCGG | | | | | |
| | | G | | | | | |
| 16S₂^{Cy5} | 22 | GCGGCCGTACTCCC | 16S rRNA (E. | Cy5 (red) | 44 | 88 | 68 |
| | | CAGGCGGTCGACTT | coli) | | | | |
| | | AACGCGTTAGCTCC | | | | | |
| | | GG | | | | | |
| 16S₃^{Cy5} | 23 | CCCCACTGCTGCCT | 16S rRNA (E. | Cy5 (red) | 49 | 87C | 63 |
| | | CCCGTAGGAGTCTG | coli) | | | | |
| | | GACCGTGTCTCAGT | | | | | |
| | | TCCAGTG | | | | | |
| 16S₄^{Cy5} | 24 | GGCAGTCTCCTTTG | 16S rRNA (E. | Cy5 (red) | 36 | 87C | 69 |
| | | AGTTCCCGGCCGGA | coli) | | | | |
| | | CCGCTGGC | | | | | |
| 16S₅^{Cy5} | 25 | CACGCGGCATGGCT | 16S rRNA (E. | Cy5 (red) | 31 | 86C | 71 |
| | | GCATCAGGCTTGCG | coli) | | | | |
| | | CCC | | | | | |
| 16S₆^{Cy5} | 26 | CGAGCTGACGACA | 16S rRNA (E. | Cy5 (red) | 42 | 86C | 64 |
| | | GCCATGCAGCACCT | coli) | | | | |
| | | GTCTCACGGTTCCC | | | | | |
| | | G | | | | | |
| 16S₇^{Cy5} | 27 | CCCCCTCCATCAGG | 16S rRNA (E. | Cy5 (red) | 45 | 85C | 62 |
| | | CAGTTTCCCAGACA | coli) | | | | |
| | | TTACTCACCCGTCC | | | | | |
| | | GCC | | | | | |

| | | | Probe range within |
|---|---|---|---|
| Gene | SEQ ID No. | Target sequence | sequence |
| SARS-CoV2 spike gene (S- | 1 | 1379-2468 | S 598-630 |
| gene) | | | S_{E} 1069-1090 |
| | | | S_{N} 43-64 |
| Influenza A H1N1 HA Gene | 7 | 482-1593 | 567-599 |
| Human 18S rRNA | 5 | 1-1869 | 205-250 |
| E. coli bacterial 16S rRNA | 31 | 1-1541 | 16S₁ 1205-1244 |
| | | | 16S₂ 855-898 |
| | | | 16S₃ 313-361 |
| | | | 16S₄ 1126-1161 |
| | | | 16S₅ 375-405 |
| | | | 16S₆ 1030-1071 |
| | | | 16S₇ 103-147 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of determining a presence of at least one target nucleic acid in a sample, the method comprising:
(a) contacting a nucleic acid-containing fraction of said sample with at least one probe compound capable of binding to said target nucleic acid, said probe compound comprises a complementary sequence;
(b) exposing said probe compound to a temperature gradient; and
(c) detecting a signal of said probe compound during said exposing to said temperature gradient,
said signal being indicative of a presence or absence of the target nucleic acid in the sample, and
the method further comprising contacting said nucleic acid-containing fraction of said sample with an additional compound capable of binding to nucleic acids.

2. The method of claim 1, wherein said detecting comprises determining a change in said signal with respect to a signal of said probe compound in the absence of said target nucleic acid, said change that is beyond a predetermined threshold of said probe compound in the absence of said target nucleic acid is indicative of a presence of the target nucleic acid in the sample.

3. The method of claim 1, wherein said determining of said change is executed quantitatively.

4. The method of claim 1, wherein said target nucleic acid is a single-stranded nucleic acid.

5. The method of claim 1, wherein said target nucleic acid comprises RNA.

6-7. (canceled)

8. The method of claim 1, wherein said probe compound comprises a nucleic acid having a sequence complementary to at least a portion of said target nucleic acid.

9. The method of claim 8, wherein said nucleic acid comprised by said probe compound comprises DNA.

10-19. (canceled)

20. The method of claim 1, further comprising contacting said nucleic acid-containing fraction of said sample with a control probe compound capable of binding to a control nucleic acid, and detecting a signal of said control probe compound during exposure to said temperature gradient.

21. The method of claim 20, comprising normalizing said signal of said probe compound to said signal of said control probe compound.

22-23. (canceled)

24. The method of claim 1, wherein said contacting with said additional compound is effected subsequently to said contacting with said probe compound.

25. The method of claim 24, wherein said additional compound capable of binding to nucleic acids comprises at least one intercalating agent.

26. The method of claim 25, wherein said intercalating agent is selected from the group consisting of an anthracycline, doxorubicin, daunorubicin, epirubicin, idarubicin, an acridine dye, proflavine, acridine orange, quinacrine, a phenanthridine, ethidium bromide, propidium iodide, berberine, dactinomycin, thalidomide, and 4′,6-diamidino-2-phenylindole (DAPI).

27-31. (canceled)

32. The method of claim 1, further comprising exposing said probe compound to a temperature of at least 80° C. prior to said contacting with said nucleic acid-containing fraction of said sample.

33. The method of claim 1, comprising concomitantly determining a presence of a first target nucleic acid in the sample using a first probe compound and a presence of a second target nucleic acid in the sample using a second probe compound.

34-36. (canceled)

37. A method of determining a presence of a target nucleic acid in a subject, comprising determining a presence of said target nucleic acid according to claim 1 in a sample obtained from the subject.

38. (canceled)

39. A method of diagnosing at least one disease or disorder in a subject, the method comprising determining a presence of at least one target nucleic acid associated with said disease or disorder according to claim 37, wherein a presence of said target nucleic acid in said biological sample is indicative of a presence of said disease or disorder in the subject.

40-43. (canceled)
